(19) United States
(12) Patent Application Publication
(10) Pub. No.: US 2014/0296257 A1
(43) Pub. Date: Oct. 2, 2014

(54) High-Loading Water-Soluable Carrier-Linked Prodrugs

(71) Applicants: Ulrich Hersel, Heidelberg (DE); Guillaume Maitro, Mannheim (DE); Harald Rau, Dossenheim (DE); Dirk Vetter, Heidelberg (DE)

(72) Inventors: Ulrich Hersel, Heidelberg (DE); Guillaume Maitro, Mannheim (DE); Harald Rau, Dossenheim (DE); Dirk Vetter, Heidelberg (DE)

(73) Assignee: Ascendis Pharma A/S, Hellerup (DK)

(21) Appl. No.: 14/237,429

(22) PCT Filed: Aug. 10, 2012
(86) PCT No.: PCT/EP2012/065731

§ 371 Date: May 13, 2014

(30) Foreign Application Priority Data
Aug. 12, 2011 (EP) 11177405.5

(51) Int. Cl. A61K 31/519; A61K 31/428; A61K 47/48

(52) U.S. Cl. CPC A61K 31/519; A61K 47/48215; A61K 31/428
USPC 514/259.41; 548/163; 544/282; 514/367

(57) ABSTRACT

The present invention relates to water-soluble carrier-linked prodrugs of formula (I), wherein B, A and Hyp form the carrier, B is a branching core, each A is independently a poly(ethylene glycol)-based polymeric chain, each Hyp is independently a branched moiety, each SP is independently a spacer moiety, each L is independently a reversible prodrug linker moiety, each D is independently a biologically active moiety, each x is independently 0 or 1, each m is independently an integer of from 2 to 64, n is an integer from 3 to 32; or the pharmaceutically acceptable salt thereof. It further relates to pharmaceutical compositions comprising said water-soluble carrier-linked prodrugs, their use as medicament or diagnostic, and methods of treatment.

The present application claims priority from PCT Patent Application No. PCT/EP2012/065731 filed on Aug. 10, 2012, which claims priority from German Patent Application No. EP 11177405.5 filed on Aug. 12, 2011, the disclosures of which are incorporated herein by reference in their entirety.

### FIELD OF THE INVENTION

It is noted that citation or identification of any document in this application is not an admission that such document is available as prior art to the present invention.

Drugs frequently exhibit short plasma half-life due to renal and receptor-mediated clearance, aggregation, proteolytic degradation, poor bioavailability and physical properties which preclude efficient formulations. This is highly undesirable as it leads to the need for frequent and repeated administration of the drug, resulting in increased costs and inconvenience for the patient.

One mechanism for enhancing the availability of drugs is by conjugating them with derivatizing compounds, which include, for example, poly(ethylene glycol) (PEG) and poly(propylene glycol). Some of the benefits recognized include lowered immunogenicity and antigenicity, increased duration of action, and altered pharmacokinetic properties (Veronese, F. M. “Enzymes for Human Therapy: Surface Structure Modifications,” Chimica Oggi, 7:53-56, 1989).

To enhance physicochemical or pharmacokinetic properties of a drug in vivo, drugs can be bound to carriers in a non-covalent way, using physicochemical formulations of drug-solvent-carrier mixtures. However, the non-covalent approach requires a highly efficient drug encapsulation to prevent uncontrolled, burst-type release of the drug. Restraining the diffusion of an unbound, water soluble drug molecule requires strong van der Waals contacts, frequently mediated through hydrophobic moieties. Many conformationally sensitive drugs, such as proteins or peptides, are rendered dysfunctional during the encapsulation process and/or during subsequent storage of the encapsulated drug. In addition, such amino-containing drugs readily undergo side reactions with carrier degradation products. Furthermore, dependence of the release mechanism of the drug upon biodegradation may cause interpatient variability.

Alternatively, the drugs may be conjugated to a carrier via a transient linker molecule, resulting in carrier-linked prodrugs. This approach is applied to various classes of molecules, from so-called small molecules, through natural products up to larger peptides and proteins.

Prodrug activation may occur by enzymatic or non-enzymatic cleavage of the bond between the carrier and the drug molecule, or a sequential combination of both, i.e. an enzymatic step followed by a non-enzymatic rearrangement.

Enzymatically induced prodrug activation is characterized in that the cleavage in enzyme-free in vitro environment such as an aqueous buffer solution, of, e.g., an ester or amide may occur, but the corresponding rate of hydrolysis may be much too slow and not therapeutically useful. In an in-vivo environment, esterases or amidases are typically present and the esterases and amidases may cause significant catalytic acceleration of the kinetics of hydrolysis from twofold up to several orders of magnitude. Therefore, the cleavage is predominantly controlled by the enzymatic reaction.

A major drawback of predominantly enzymatic cleavage is interpatient variability. Enzyme levels may differ significantly between individuals resulting in biological variation of prodrug activation by the enzymatic cleavage. The enzyme levels may also vary depending on the site of administration. For instance it is known that in the case of subcutaneous injection, certain areas of the body yield more predictable therapeutic effects than others. To reduce this unpredictable effect, non-enzymatic cleavage or intramolecular catalysis is of particular interest.

Therefore, enzyme-independent autocatalytic cleavage of carrier and drug is preferred. In most cases this is achieved by an appropriately designed linker moiety between the carrier and the drug, which is directly attached to a functional group of a drug via covalent bond.

A number of such enzyme-independent prodrugs suitable for different classes of biologically active moieties are known in the art. Examples can be found in the international patent applications WO-A 2005/099768, WO-A 2006/13565869, WO-A 2009/095479, and WO-A 2011/012722.

Typically, carrier-linked prodrugs have a stoichiometry of one drug molecule conjugated to one carrier moiety. However, for many medical applications such stoichiometry is disadvantageous as large volumes of such conjugates would have to be applied to a patient to ensure a high enough dose of drug, causing undue pain and possibly requiring increased amounts of time for the administration process and thus increasing the costs of the treatment. In such situations, carrier-linked prodrugs in which more than one drug moiety is conjugated to a carrier molecule might be better suited as they provide a higher drug loading and thus require smaller volumes of the pharmaceutical composition to be administered to a patient.

Patent U.S. Pat. No. 7,744,861 B2 discloses multi-arm prodrugs in which at least three arms extend from a branching core and each of these arms carries one drug moiety. Similarly, WO-A 2010/019233 discloses multi-arm prodrugs of which each arm of a carrier moiety is conjugated to one drug moiety. Despite the multi-arm backbone structure, such carrier-linked prodrugs still have a relatively low drug load.

More carrier-linked prodrugs with two polymer-based arms are disclosed in WO-A 2008/034119, wherein each arm is attached to a drug moiety, diagnostic agent or targeting moiety.

Prodrugs of the anti-malaria drug artelinic acid are disclosed in U.S. Pat. No. 6,461,603 B2. The polymeric prodrugs are also based on a backbone moiety from which arms extend which each carry one drug moiety at their terminus.

Another approach to high-loading carrier-linked prodrugs involves the use of dendrimers. Dendrimers are repeatedly branched, roughly spherical, large molecules. Dendrimers have been used to non-covalently embed drug moieties and for covalent attachment of drug moieties to the termini of the dendrimer.

Taite & West (J. Biomater. Sci. Polymer Edn, 2006, 17, 1159-1172) describe lysine-based dendrimer moieties in which free amines have been converted to diazeniumdiolate NO-donors through the reaction with NO gas. The dendrimers released NO over a period of 60 days. However, this approach does not allow for the adjustment of release speed as no reversible prodrug linkers have been used to attach the NO to the termini of the dendrimer and this approach is also not transferable to other drug moieties.

US 2010/0160299 A1 discloses dendrimers to which therapeutic agents for the reduction and/or elimination of pain are connected in a reversible manner. Similarly, WO-A 2010/075423 discloses modular dendrimer platforms suitable for the delivery of therapeutic agents, for example.

However, dendrimers typically exhibit a low degree of water-solubility. When poorly water-soluble drug moieties are coupled to the functional groups of such dendrimers the resulting conjugates are even less water-soluble. Therefore, although dendrimers provide a high drug loading, their applicability for prodrug approaches is limited.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right to disclaim, and hereby disclose a disclaimer of, any previously described product, method of making the product, or process of using the product.

### SUMMARY OF THE INVENTION

Therefore, there is a need to provide novel water-soluble carrier-linked prodrugs that at least partially overcome the above-mentioned shortcomings. This object is achieved with a water-soluble carrier-linked prodrug of formula (I):

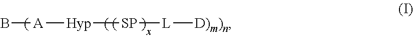

wherein
B, A and Hyp form a carrier moiety, and wherein
   B is a branching core,
   each A is independently a poly(ethylene glycol)-based polymeric chain,
   each Hyp is independently a branched moiety,
   each SP is independently a spacer moiety,
   each L is independently a reversible prodrug linker moiety,
   each D is independently a biologically active moiety,
   each x is independently 0 or 1,
   each m is independently an integer of from 2 to 64,
   n is an integer from 3 to 32;
or a pharmaceutically acceptable salt thereof.

It was now surprisingly found that such water-soluble carrier-linked prodrug can be used as a sustained-release dosage form of biologically active moieties with a high drug loading due to the presence of the branched moieties. In addition, the poly(ethylene glycol)-based (PEG-based) polymeric chain allows for increased water-solubility.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

Within the present invention the terms are used having the meaning as follows.

The terms “drug”, “biologically active molecule”, “biologically active moiety”, “biologically active agent”, “active agent”, “active substance” and the like mean any substance which can affect any physical or biochemical properties of a biological organism, including but not limited to viruses, bacteria, fungi, plants, animals, and humans. In particular, as used herein, the terms include any substance intended for diagnosis, cure, mitigation, treatment, or prevention of disease in organisms, in particular humans or other animals, or to otherwise enhance physical or mental well-being of organisms, in particular humans or animals.

“Biologically active moiety D” means the part of a biologically active moiety-reversible prodrug linker conjugate or the part of a biologically active moiety-reversible prodrug linker-carrier conjugate, which results after cleavage in a drug D-H of known biological activity.

“Amine-containing biologically active moiety” or “hydroxyl-containing biologically active moiety” means the part (moiety or fragment) of a biologically active moiety-reversible prodrug linker conjugate or the part of a biologically active moiety-reversible prodrug linker-carrier conjugate (active agent) of (known) biological activity, and which part of the drug comprises at least one amine or hydroxyl group, respectively.

In addition, the subterm “aromatic amine-containing” means that the respective biologically active moiety D and analogously the corresponding drug D-H contains at least one aromatic fragment which is substituted with at least one amino group. The subterm “aliphatic amine-containing” means that the respective biologically active moiety D and analogously the corresponding drug D-H contains at least one aliphatic fragment which is substituted with at least one amino group. Without further specification the term “amine-containing” is used generically and refers to aliphatic and aromatic amine-containing moieties.

The subterm “aromatic hydroxyl-containing” means that the respective moiety D and analogously the corresponding drug D-H contains at least one aromatic fragment, which is substituted with at least one hydroxyl group. The subterm “aliphatic hydroxyl-containing” means that the hydroxyl group of the respective moiety D and analogously the corresponding drug D-H is connected to an aliphatic fragment. Without further specification the term “hydroxyl-containing” is used generically and refers to aliphatic and aromatic hydroxyl-containing moieties.

“Free form” of a drug refers to the drug in its unmodified, pharmacologically active form, such as after being released from a carrier-linked prodrug.

Targeting moieties are moieties that when present in a molecule, such as for example in a prodrug, allow preferential localization of such larger molecule in specific target areas of the organism to which it has been administered. Such specific target areas might be organs, certain cell types or subcellular compartments. “Preferential localization” means that at least 10%, preferably at least 20% and more preferably at least 30% of the biologically active moieties administered to a patient reach said specific target areas.

Targeting moieties may be divided into 3 classes according to size:
small molecular targeting moieties, for example C-glucuronide, cobalamin, vitamins such as folic acid (folate) and analogs and derivatives, carbohydrates, bisphosphonates, N-acetylgalactosamine,
peptides, for example bombesin, somatostatin, LHRH, EGF, VEGF, hCG, fragments of luteinizing hormone (LH), octreotide, vapreotide, lanreotide, RC-3940 series, decapeptyl, lupron, zoladex, cetrorelix, peptides or peptidomimetics containing the NGR or RGD motifs or derived from these motifs such as CNGRC (linear), GNGRG (cyclic), ACDC RGD CFCG (cyclic), CDCRGDCFC, CNGRC (cyclic), CRGDCGG, CNGRC, or other peptides such as ATWLPPR, thrombospondin (TSP)-1 mimetics, (RGD peptidomimetic), CTTHWGFTLC, CGNKRTRGC, neuropeptide substance P, SSP, the Sar9, Met(O2)11 analog of substance P, cholecystokinin (CCK), corticotropin-releasing hormone/factor (CRH/CRF), dermorphin, FGF-2 or basic fibroblast growth factor, galanin, melanopsin, neurotensin,
and protein or macro-molecular targeting moieties, for example IL-2, GM-CSF, TNF-a, transferrin, immunoglobulins, acetylated-LDL, lactoferrin (Lf) (also called lactotransferrin) and lactoferricin (Lcin), gambogic acid (GA), antibody fragments and affinity scaffold proteins.

In principle, any ligand of a cell surface receptor may be advantageously used as a targeting moiety. For instance, ATWLPPR peptide is a potent antagonist of VEGF; thrombospondin-1 (TSP-1) induces apoptosis in endothelial cells, RGD-motif mimics block integrin receptors, NGR-containing peptides inhibit aminopeptidase N, and cyclic peptides containing the sequence of HWGF selectively inhibit MMP-2 and MMP-9. LyP-1 peptide specifically binds to tumor lymphatic vessels. Illustrative other ligands include peptide ligands identified from library screens, tumor cell-specific peptides, tumor cell-specific aptamers, tumor cell-specific carbohydrates, tumor cell-specific monoclonal or polyclonal antibodies, Fab or scFv (i.e., a single chain variable region) fragments of antibodies such as, for example, a Fab fragment of an antibody directed to EphA2 or other proteins specifically expressed or uniquely accessible on metastatic cancer cells, small organic molecules derived from combinatorial libraries, growth factors, such as EGF, FGF, insulin, and insulin-like growth factors, and homologous polypeptides, somatostatin and its analogs, transferrin, lipoprotein complexes, bile salts, selecting, steroid hormones, Arg-Gly-Asp containing peptides, retinoids, various Galectins, δ-opioid receptor ligands, cholecystokinin A receptor ligands, ligands specific for angiotensin AT1 or AT2 receptors, peroxisome proliferator-activated receptor λ ligands, β-lactam antibiotics such as penicillin, small organic molecules including antimicrobial drugs, and other molecules that bind specifically to a receptor preferentially expressed on the surface of tumor cells or on an infectious organism, antimicrobial and other drugs designed to fit into the binding pocket of a particular receptor based on the crystal structure of the receptor or other cell surface protein, ligands of tumor antigens or other molecules preferentially expressed on the surface of tumor cells, or fragments of any of these molecules. Examples of tumor-specific antigens that can function as targeting moieties include extracellular epitopes of a member of the ephrin family of proteins, such as EphA2. EphA2 expression is restricted to cell-cell junctions in normal cells, but EpbA2 is distributed over the entire cell surface in metastatic tumor cells. Thus, EphA2 on metastatic cells would be accessible for binding to, for example, a Fab fragment of an antibody conjugated to an immunogen, whereas the protein would not be accessible for binding to the Fab fragment on normal cells, resulting in a targeting moiety specific for metastatic cancer cells.

Further examples for such targeting moieties are: FSH-33, allatostatin 1, hepatocarcinoma targeting peptide, peptide GFE, anti-EGFR antibodies and/or antibody fragments, in particular cetuximab, CendR, iRGD peptide (RGD-CendR hybrid peptide), small molecules, antibodies and/or antibody fragments binding to cancer-specific epitopes like e.g. CEA, gastrin-releasing peptide receptors, somatostatin receptors, galanin receptors, follicle-stimulating hormone receptors, p32 protein, fibroblast growth factor receptors, HepG2, epidermal growth factor receptors, integrin αvβ6, neuropilin-1 receptor and VEGF receptors.

The phrases “in bound form”, “connected to”, and“moiety” refer to sub-structures which are part of a molecule. The phrases “in bound form” or “connected to” are used to simplify reference to moieties or functional groups by naming or listing reagents, starting materials or hypothetical starting materials well known in the art, and whereby “in bound form” and “connected to” means that for example one or more hydrogen radicals (—H) or one or more activating or protecting groups present in the reagents or starting materials are not present in the moiety when part of a molecule.

To enhance physicochemical or pharmacokinetic properties of a drug in vivo, such drug can be conjugated with a carrier, as in the present invention. If the drug is transiently bound to a carrier and/or a linker, as in the present invention, such systems are commonly assigned as “carrier-linked prodrugs”. According to the definitions provided by IUPAC (as given under http://www.chem.qmul.ac.uk/iupac/medchem/ah.html, accessed on Mar. 7, 2011), a carrier-linked prodrug is a prodrug that contains a temporary linkage of a given active substance with a transient carrier group that produces improved physicochemical or pharmacokinetic properties and that can be easily removed in vivo, usually by a hydrolytic cleavage.

The term “promoiety” refers to the part of the prodrug which is not the drug, thus meaning linker(s), carrier(s) and/or any optional spacer moiety/moieties.

The terms “reversible prodrug linker” or “transient prodrug linker” refer to linker that are non-enzymatically hydrolytically degradable, i.e. cleavable, under physiological conditions (aqueous buffer at pH 7.4, 37° C.) with half-lives ranging from, for example, one hour to three months. On the other hand, stable or permanent linkers have stable or permanent linkages, which are typically non-cleavable permanent bonds, meaning that they have a half-life of at least six months under physiological conditions (aqueous buffer at pH 7.4, 37° C.).

A “traceless prodrug linker” refers to a prodrug linker from which a drug is released in its free form, meaning that upon release from the promoiety the drug does not contain any traces of the promoiety. “Non-biologically active linker” means a linker which does not show the pharmacological effects of the drug (D-H) derived from the biologically active moiety.

The term “polymer” describes a molecule comprising, in particular consisting of, repeating structural units connected by chemical bonds in a linear, circular, branched, crosslinked or dendrimeric way or a combination thereof, which can be of synthetic or biological origin or a combination of both. It is understood, that e.g. capping moieties may be present in a polymer.

The term “polymeric” refers to a moiety comprising one or more polymer.

The term “poly(ethylene glycol)-based polymeric chain” or “PEG-based polymeric chain” refers to a polymer chain comprising at least 10% by weight, preferably at least 25%, more preferably at least 50% by weight, even more preferably at least 80% by weight poly(ethylene glycol). It is understood that a PEG-based polymeric chain may be terminated in case of branched chains and/or or interrupted by alkyl or aryl groups and optionally be substituted with heteroatoms and/or functional groups.

The terms “spacer”, “spacer group”, “spacer molecule”, and “spacer moiety” are used interchangeably and refer to any moiety suitable for connecting two moieties, such as C₁₋₅₀ alkyl, C₂₋₅₀ alkenyl or C₂₋₅₀ alkinyl, which moiety is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl and naphthyl.

The term “terminus” refers to the last carbon atom or heteroatom of a linear or branched chain comprising, in particular consisting of carbon atoms or heteroatoms, i.e. to a carbon or heteroatom which is connected to exactly one other carbon or heteroatom.

The term “branched moiety” refers to a moiety comprising at least one branching point. Such branching point comprises, for example, an at least 3-fold substituted carbocycle, an at least 3-fold substituted heterocycle, a tertiary carbon atom, a quaternary carbon atom or a tertiary nitrogen atom.

A carbocycle and heterocycle may be substituted by C₁₋₂₀ alkyl, optionally interrupted or terminated by heteroatoms or functional groups selected from the group consisting of —O—, —S—, N(R), C(O), C(O)N(R), and N(R)C(O), wherein R is hydrogen or a C₁₋₁₀ alkyl chain, which is optionally interrupted or terminated by one or more of the above mentioned atoms or groups which further have a hydrogen as terminal atom.

“Pharmaceutical composition” or “composition” means a composition comprising one or more drugs or prodrugs, and optionally one or more pharmaceutically acceptable excipients, as well as any product which results, directly or indirectly, from combination, complexation or aggregation of any two or more of the ingredients, or from dissociation of one or more of the pharmaceutically acceptable excipients, or from other types of reactions or interactions of one or more of the pharmaceutically acceptable excipients. Accordingly, the pharmaceutical compositions of the present invention encompass any composition obtainable by admixing a water-soluble carrier-linked prodrug of the present invention and optionally one or more pharmaceutically acceptable excipients.

The term “excipient” refers to a diluent, adjuvant, or vehicle with which the water-soluble carrier-linked prodrug is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These compositions can take the form of solutions, suspensions, emulsions, tablets, pills, capsules, powders, sustained-release formulations and the like. The composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in “Remington's Pharmaceutical Sciences” by E. W. Martin. Such compositions will contain a diagnostically and/or therapeutically effective amount of the a water-soluble carrier-linked prodrug, preferably in purified form, together with a suitable amount of excipient so as to provide the form for proper administration to the patient. The formulation should suit the mode of administration.

The term “pharmaceutically acceptable” means approved by a regulatory agency such as the EMEA (Europe) and/or the FDA (US) and/or any other national regulatory agency for use in animals, preferably in humans.

“Dry composition” means that the pharmaceutical composition comprising water-soluble carrier-linked prodrug according to the present invention is provided in a dry form in a container. Suitable methods for drying are spray-drying and lyophilization (freeze-drying). Such dry composition of water-soluble carrier-linked prodrug has a residual water content of a maximum of 10%, preferably less than 5% and more preferably less than 2% (determined according to Karl Fischer). The preferred method of drying is lyophilization.

“Lyophilized composition” means that the pharmaceutical composition comprising water-soluble carrier-linked prodrug was first frozen and subsequently subjected to water reduction by means of reduced pressure. This terminology does not exclude additional drying steps which may occur in the manufacturing process prior to filling the composition into the final container.

“Lyophilization” (freeze-drying) is a dehydration process, characterized by freezing a composition and then reducing the surrounding pressure and, optionally, adding heat to allow the frozen water in the composition to sublime directly from the solid phase to gas. Typically, the sublimed water is collected by desublimation.

The term “functional group” refers to a specific group of atoms within molecules that can undergo characteristic chemical reactions. Examples of functional groups are hydroxyl, carbonyl, aldehyde, carboxyl, ester, ketal, hemiketal, acetal, hemiacetal, primary/secondary/tertiary amine, cyanate, disulfide, sulfhydryl, sulfonyl and phosphate groups.

If a functional group is coupled to another functional group, the resulting chemical structure is referred to as “linkage”. For example, the reaction of an amine functional group with a carboxyl functional group results in an amide linkage. Further examples for linkages are ester, ether, ketal, acetal, secondary/tertiary amine, carboxamide, sulfide and disulfide linkages.

“Alkyl” means a straight-chain or branched carbon chain (unsubstituted alkyl). Optionally, one or more hydrogen atoms of an alkyl carbon may be replaced by a substituent. In general, a preferred alkyl is C₁₋₆ alkyl.

“C₁₋₄ alkyl” means an alkyl chain having 1 to 4 carbon atoms (unsubstituted C₁₋₄ alkyl), e.g. if present at the end of a molecule: methyl, ethyl, n-propyl, isopropyl, n-butyl, isobutyl, sec-butyl tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group (also referred to as C₁₋₄ alkylene). Optionally, one or more hydrogen atom(s) of a C₁₋₄ alkyl carbon may be replaced by a substituent as indicated herein. Accordingly, “C₁₋₅₀ alkyl” means an alkyl chain having 1 to 50 carbon atoms.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl, tert-butyl, n-pentyl, n-hexyl, or e.g. —CH₂—, —CH₂—CH₂—, —CH(CH₃)—, —C(CH₂)—, —CH₂—CH₂—CH₂—, —CH(C₂H₅)—, —C(CH₃)₂—, when two moieties of a molecule are linked by the alkyl group (also referred to as C₁₋₆ alkylene). One or more hydrogen atom(s) of a C₁₋₆ alkyl carbon may be replaced by a substituent as indicated herein. The terms C₁₋₁₅ alkyl or C₁₋₁₅ alkylene are defined accordingly.

“C₂₋₆ alkenyl” means an alkenyl chain having 2 to 6 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. One or more hydrogen atom(s) of a C₂₋₆ alkenyl carbon may be replaced by a substituent as indicated herein.

The term C₂₋₄ alkenyl is defined accordingly.

“C₂₋₆ alkynyl” means an alkynyl chain having 2 to 6 carbon atoms, e.g. if present at the end of a molecule: —C≡CH, —CH₂—C≡CH, CH₂—CH₂—C≡CH, CH₂—C≡C—CH₃, or e.g. —C≡C— when two moieties of a molecule are linked by the alkynyl group. One or more hydrogen atom(s) of a C₂₋₆ alkynyl carbon may be replaced by a substituent as indicated herein. The term C₂₋₄ alkynyl is defined accordingly.

“C₂₋₅₀ alkenyl” means a branched or unbranched alkenyl chain having 2 to 50 carbon atoms (unsubstituted C₂₋₅₀ alkenyl), e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Optionally, one or more hydrogen atom(s) of a C₂₋₅₀ alkenyl carbon may be replaced by a substituent as further specified. Accordingly, the term “alkenyl” relates to a carbon chain with at least one carbon double bond. Optionally, one or more triple bonds may occur. The term “C₂₋₁₅ alkenyl” is defined accordingly.

“C₂₋₅₀ alkynyl” means a branched or unbranched alkynyl chain having 2 to 50 carbon atoms (unsubstituted C₂₋₅₀ alkynyl), e.g. if present at the end of a molecule: —C≡CH, —CH₂—C≡CH, CH₂—CH₂—C≡CH, CH₂—C≡C—CH₃, or e.g. —C≡C— when two moieties of a molecule are linked by the alkynyl group. Optionally, one or more hydrogen atom(s) of a C₂₋₅₀ alkynyl carbon may be replaced by a substituent as further specified. Accordingly, the term “alkynyl” relates to a carbon chain with at least one carbon triple bond. Optionally, one or more double bonds may occur.

“C₃₋₇ cycloalkyl” or “C₃₋₇ cycloalkyl ring” means a cyclic alkyl chain having 3 to 7 carbon atoms, which may have carbon-carbon double bonds being at least partially saturated (unsubstituted C₃₋₇ cycloalkyl), e.g. cyclopropyl, cyclobutyl, cyclopentyl, cyclohexyl, cyclohexenyl, cycloheptyl. Optionally, one or more hydrogen atom(s) of a cycloalkyl carbon may be replaced by a substituent as indicated herein. The term “C₃₋₇ cycloalkyl” or “C₃₋₇ cycloalkyl ring” also includes bridged bicycles like norbonane (norbonanyl) or norbonene (norbonenyl). Accordingly, “C₃₋₅ cycloalkyl” means a cycloalkyl having 3 to 5 carbon atoms. Accordingly, “C₃₋₁₀ cycloalkyl” means a cycloalkyl having 3 to 10 carbon atoms.

“Halogen” means fluoro, chloro, bromo or iodo. It is generally preferred that halogen is fluoro or chloro.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom (unsubstituted 4 to 7 membered heterocyclyl). For the sake of completeness it is indicated that in some embodiments of the present invention, 4 to 7 membered heterocyclyl has to fulfill additional requirements. Examples for a 4 to 7 membered heterocycles are azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine. Optionally, one or more hydrogen atom(s) of a 4 to 7 membered heterocyclyl may be replaced by a substituent.

“8 to 11 membered heterobicyclyl” or “8 to 11 membered heterobicycle” means a heterocyclic system of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom (unsubstituted 8 to 11 membered heterobicyclyl). Examples for a 8 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine or pteridine. The term 8 to 11 membered heterobicycle also includes spiro structures of two rings like 1,4-dioxa-8-azaspiro[4.5]decane or bridged heterocycles like 8-aza-bicyclo[3.2.1]octane. The term “9 to 11 membered heterobicyclyl” or “9 to 11 membered heterobicycle” is defined accordingly.

The term “aliphatic” means fully saturated.

The term “interrupted” means that between two carbon atoms of, for example, a linker or a spacer or at the respective end of the carbon chain between the respective carbon atom and the hydrogen atom a group (such a —O— or —NH—) is inserted.

In general the term “substituted” preferably refers to substituents, which are the same or different and which are independently selected from the group consisting of halogen, CN, COOR^{b9}, OR^{b9}, C(O)R^{b9}, C(O)N(R^{b9}R^{b9a}), S(O)₂N(R^{b9}R^{b9a}), S(O)N(R^{b9}R^{b9a}), S(O)₂R^{b9}, S(O)R^{b9}, N(R^{b9})S(O)₂N(R^{b9a}R^{b9b}), SR^{b9}, N(R^{b9}R^{b9a}), NO₂, OC(O)R^{b9}, N(R^{b9})C(O)R^{b9a}, N(R^{b9})S(O)₂R^{b9a}, N(R^{b9})S(O)R^{b9a}, N(R^{b9})C(O)OR^{b9a}, N(R^{b9})C(O)N(R^{b9a}R^{b9b}), OC(O)N(R^{b9}R^{b9a}), T^{b}, C₁₋₅₀ alkyl, C₂₋₅₀ alkenyl, and C₂₋₅₀ alkynyl,
wherein T^{b}, C₁₋₅₀ alkyl, C₂₋₅₀ alkenyl, and C₂₋₅₀ alkynyl are optionally substituted with one or more R^{b10}, which are the same or different, and wherein C₁₋₅₀ alkyl; C₂₋₅₀ alkenyl; and C₂₋₅₀ alkynyl are optionally interrupted by one or more groups selected from the group consisting of T^{b}, —C(O)O—; —O—; —C(O)—; —C(O)N(R^{b11})—; —S(O)₂N(R^{b11})—; —S(O)N(R^{b11})—; —S(O)₂—; —S(O)—; —N(R^{b11})S(O)₂N(R^{b11a})—; —S—; —N(R^{b11})—; —OC(O)R^{b11}; —N(R^{b11})C(O)—; —N(R^{b11})S(O)₂—; —N(R^{b11})S(O)—; —N(R^{b11})C(O)O—; —N(R^{b11})C(O)N(R^{b11a})—; and —OC(O)N(R^{b11}R^{b11a});
R^{b9}, R^{b9a}, R^{b9b} are independently selected from the group consisting of H; T^{b}; and C₁₋₅₀ alkyl; C₂₋₅₀ alkenyl; and C₂₋₅₀ alkynyl,
   wherein T^{b}, C₁₋₅₀ alkyl, C₂₋₅₀ alkenyl, and C₂₋₅₀ alkynyl are optionally substituted with one or more R^{b10}, which are the same or different, and wherein C₁₋₅₀ alkyl; C₂₋₅₀ alkenyl; and C₂₋₅₀ alkynyl are optionally interrupted by one or more groups selected from the group consisting of T^{b}, —C(O)O—, —O—, —C(O)—, —C(O)N(R^{b11})—, —S(O)₂N(R^{b11})—, —S(O)N(R^{b11})—, —S(O)₂—, —S(O)—, —N(R^{b11})S(O)₂N(R^{b11a})—, —S—, —N(R^{b11})—, —OC(O)R^{b11}, —N(R^{b11})C(O)—, —N(R^{b11})S(O)₂—, —N(R^{b11})S(O)—, —N(R^{b11})C(O)O—, —N(R^{b11})C(O)N(R^{b11a})—, and —OC(O)N(R^{b11}R^{b11a}),
   T^{b} is selected from the group consisting of phenyl, naphthyl, indenyl, indanyl, tetralinyl, C₃₋₁₀ cycloalkyl, 4- to 7-membered heterocyclyl, and 9- to 11-membered heterobicyclyl, wherein T^{b} is optionally substituted with one or more R^{b10}, which are the same or different,
   R^{b10} is halogen, CN, oxo (═O), COOR^{b12}, OR^{b12}, C(O)R^{b12}, C(O)N(R^{b12}R^{b12a}), S(O)₂N(R^{b12}R^{b12a}), S(O)N(R^{b12}R^{b12a}), S(O)₂R^{b12}, S(O)R^{b12}, N(R^{b12})S(O)₂N(R^{b12a}R^{b12b}), SR^{b12}, N(R^{b12}R^{b12a}), NO₂, OC(O)R^{b12}N(R^{b12})C(O)R^{b12a}, N(R^{b12})S(O)₂R^{b12a}, N(R^{b12})S(O)R^{b12a}, N(R^{b12})C(O)OR^{b12a}, N(R^{b12})C(O)N(R^{b12a}R^{b12b}), OC(O)N(R^{b12}R^{b12a}), or C₁₋₆ alkyl, wherein C₁₋₆ alkyl is optionally substituted with one or more halogen, which are the same or different,
   R^{b11}, R^{b11a}, R^{b12}, R^{b12a}, R^{b12b} are independently selected from the group consisting of H; or C₁₋₆ alkyl, wherein C₁₋₆ alkyl is optionally substituted with one or more halogen, which are the same or different.

The term “interrupted” means that between two carbons a group is inserted or that at the end of the carbon chain between the carbon and hydrogen.

In general the term “comprise” or “comprising” also encompasses “consist of” or “consisting of”.

In the following section the invention is described in further detail.

The present invention refers to a water-soluble carrier-linked prodrug of formula (I):

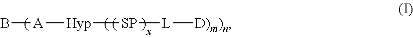

wherein
B, A and Hyp form a carrier moiety, and wherein
   B is a branching core,
   each A is independently a poly(ethylene glycol)-based polymeric chain,
   each Hyp is independently a branched moiety,
   each SP is independently a spacer moiety,
   each L is independently a reversible prodrug linker moiety,
   each D is independently a biologically active moiety,
   each x is independently 0 or 1,
   each m is independently an integer of from 2 to 64,
   n is an integer from 3 to 32;
or a pharmaceutically acceptable salt thereof.

The moieties A of the water-soluble carrier-linked prodrug of formula (I) may be the same or different. Preferably all moieties A of formula (I) are the same.

The moieties Hyp of the water-soluble carrier-linked prodrug of formula (I) may be the same or different. Preferably all moieties Hyp of formula (I) are the same.

The moieties SP of the water-soluble carrier-linked prodrug of formula (I) may be the same or different. Preferably all moieties SP of formula (I) are the same.

The moieties L of the water-soluble carrier-linked prodrug of formula (I) may be the same or different. Preferably all moieties L of formula (I) are the same.

The moieties D of the water-soluble carrier-linked prodrug of formula (I) may be the same or different. Preferably all moieties D of formula (I) are the same.

Each n of the water-soluble carrier-linked prodrug of formula (I) may be the same or different. Preferably all n of formula (I) are the same.

Each x of the water-soluble carrier-linked prodrug of formula (I) may be the same or different. Preferably all x of formula (I) are the same.

Preferably, all n, x and all moieties A, Hyp, SP, L, D of the water-soluble carrier-linked prodrug of formula (I) are the same.

It is understood that m is equal to or less than the number of functional groups of Hyp of formula (I).

Preferably, m is an integer from 2 to 32, more preferably from 2 to 24, more preferably from 2 to 12, more preferably m is 2, 3, 4, 5, 6, 7, 8, 9, or 10, and even more preferably m is 2, 3, 4, 5, 6, 7, or 8. Most preferably, m is 2.

In a preferred embodiment, the branching core B of formula (I) comprises, preferably consists of, a moiety selected from:
a polyalcohol comprising at least 2 hydroxyl groups (preferably further comprising a functional group, which is preferably an additional amino group or a carboxylic acid group, more preferably an additional carboxylic acid group),
preferably B is selected from glycerol, pentaerythritol, dipentaerythritol, tripentaerythritol, hexaglycerine, sucrose, sorbitol, fructose, mannitol, glucose, cellulose, amyloses, starches, hydroxyalkyl starches, polyvinylalcohols, dextranes, and hyualuronans,
or a polyamine comprising at least 2 amine groups (preferably further comprising a functional group, which is preferably an additional hydroxyl group or a carboxylic acid group, more preferably a carboxylic acid group),
preferably selected from ornithine, diornithine, triornithine, tetraomithine, pentaornithine, hexaornithine, heptaornithine, octaornithine, nonaornithine, decaornithine, undecaornithine, dodecaornithine, tridecaornithine, tetradecaornithine, pentadecaornithine, hexadecaornithine, heptadecaornithine, octadecaornithine, nonadecaornithine, diaminobutyric acid, di(diaminobutyric acid), tri(diaminobutyric acid), tetra(diaminobutyric acid), penta(diaminobutyric acid), hexa(diaminobutyric acid), hepta(diaminobutyric acid), octa(diaminobutyric acid), nona(diaminobutyric acid), deca(diaminobutyric acid), undeca(diaminobutyric acid), dodeca(diaminobutyric acid), trideca(diaminobutyric acid), tetradeca(diaminobutyric acid), pentadeca(diaminobutyric acid), hexadeca(diaminobutyric acid), heptadeca(diaminobutyric acid), octadeca(diaminobutyric acid), nonadeca(diaminobutyric acid), lysine, dilysine, trilysine, tetralysine, pentalysine, hexalysine, heptalysine, octalysine, nonalysine, decalysine, undecalysine, dodecalysine, tridecalysine, tetradecalysine, pentadecalysine, hexadecalysine, heptadecalysine, octadecalysine, nonadecalysine, oligolysines, polyethyleneimines, and polyvinylamines;
wherein the polyalcohol or polyamine is in bound form.

In a preferred embodiment, the branching core B comprises, preferably consists of, pentaerythritol.

Preferably, each A of formula (I) individually consists of a linear PEG-based chain, of which one terminus is connected to B and the other terminus is connected to a moiety Hyp. It is understood that each moiety A of formula (I) may independently optionally be terminated in case of a branched PEG-based chain and/or may optionally be interrupted in case of a branched or linear PEG-based chain by alkyl or aryl groups and may optionally be substituted with heteroatoms and/or functional groups.

According to the present invention, each A and each Hyp may be selected independently from the other moieties A and Hyp of formula (I). Preferably, all moieties A of formula (I) are the same and all moieties Hyp of formula (I) are the same.

According to the present invention, each sub-structure A-Hyp of formula (I) may be independently the same or a different sub-structure A-Hyp. In a preferred embodiment, all sub-structures A-Hyp of formula (I) are the same.

Preferably, each moiety A of formula (I) is connected to B through a permanent linkage.

According to the invention, n is an integer from 3 to 32. Preferably, n is an integer from 3 to 16, more preferably n is an integer from 4 to 8 and most preferably n is 4.

In a preferred embodiment n is 4 and m is 2.

In one embodiment, each moiety A is independently selected from linear and branched poly(ethylene glycol)-based polymeric chains. Preferably, each A is independently a linear poly(ethylene glycol)-based polymeric chain.

Preferably, each A is independently selected from the formula

—X3-(CH₂)ₙ₁—(OCH₂CH₂)ₚ—O—(CH₂)ₙ₂—X2,
wherein
n1 and n2 are independently 1, 2, 3, or 4, preferably n1 and n2 are independently 1, 2, or 3, more preferably 2 or 3;
p is an integer from 5 to 2000, preferably p is an integer from 10 to 1000, more preferably from 20 to 1000, more preferably, 50 to 1000 and more preferably p is an integer from 100 to 1000; and
X3 is a chemical bond or linkage group covalently linked to B, and
X2 is a chemical bond or linkage group covalently linked to Hyp.

Preferably, each linkage between a moiety A and a moiety Hyp of formula (I) is a permanent linkage, more preferably a permanent linkage comprising, preferably consisting of, a linkage group selected from amine, amide, carbamate, thioether, or ether groups, and most preferably each permanent linkage between A and Hyp of formula (I) is an amide linkage, i.e. X2 is an amide linkage (—NH—(C═O)—).

In a preferred embodiment, a sub-structure B-(A)ₙ of formula (I) is a multi-arm PEG derivative as, for instance, detailed in the products list of JenKem Technology, USA (accessed by download from http://jenkemusa.net/pegproducts2.aspx on Mar. 8, 2011), such as a 4-arm-PEG derivative, in particular comprising a pentaerythritol core, an 8-arm-PEG derivative comprising a hexaglycerin core, and an 8-arm-PEG derivative comprising a tripentaerythritol core. Most preferred are sub-structures B-(A)ₙ of formula (I) comprising, in particular consisting of, moieties selected from:

a 4-arm PEG Amine comprising a pentaerythritol core:

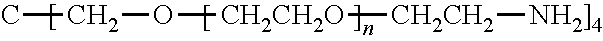

with n ranging from 400 to 2000;

a 4-arm PEG Carboxyl comprising a pentaerythritol core:

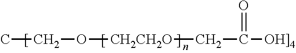

with n ranging from 400 to 2000;

an 8-arm PEG Amine comprising a hexaglycerin core:

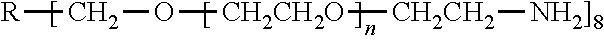

with n ranging from 400 to 2000 and

R=hexaglycerin core structure;

an 8-arm PEG Carboxyl comprising a hexaglycerin core:

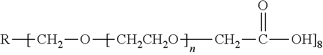

with n ranging from 400 to 2000 and

R=hexaglycerin core structure;

an 8-arm PEG Amine comprising a tripentaerythritol core:

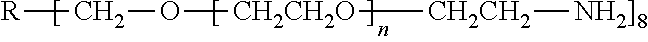

with n ranging from 400 to 2000

and R=tripentaerythritol core structure;

and an 8-arm PEG Carboxyl comprising a tripentaerythritol core:

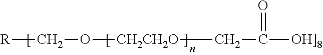

with n ranging from 400 to 2000 and

R=tripentaerythritol core structure;

each in bound form.

In a preferred embodiment, the molecular weight of the sub-structure B-(A)ₙ of formula (I) ranges from 1 kDa to 160 kDa, more preferably from 1 kDa to 80 kDa and even more preferably from 10 kDa to 40 kDa. It is understood that the terminal amine groups or carboxyl groups, respectively, are used for conjugation to Hyp of formula (I).

In a preferred embodiment, a moiety Hyp of the water-soluble carrier-linked prodrug of formula (I) comprises, preferably consists of, a moiety selected from
a polyalcohol in bound form comprising at least 2 hydroxyl groups (preferably further comprising a functional group, which is preferably an additional hydroxyl group or a carboxylic acid group, more preferably an additional hydroxyl group),
preferably selected from glycerol, pentaerythritol, dipentaerythritol, tripentaerythritol, hexaglycerine, sucrose, sorbitol, fructose, mannitol, glucose, cellulose, amyloses, starches, hydroxyalkyl starches, polyvinylalcohols, dextranes, and hyualuronans,
or a polyamine in bound form comprising at least 2 amine groups (preferably further comprising a functional group, which is preferably an additional amine group or a carboxylic acid group, more preferably a carboxylic acid group),
preferably selected from ornithine, diornithine, triornithine, tetraornithine, pentaornithine, hexaornithine, heptaornithine, octaornithine, nonaornithine, decaornithine, undecaornithine, dodecaornithine, tridecaornithine, tetradecaornithine, pentadecaornithine, hexadecaornithine, heptadecaornithine, octadecaornithine, nonadecaornithine, diaminobutyric acid, di(diaminobutyric acid), tri(diaminobutyric acid), tetra(diaminobutyric acid), penta(diaminobutyric acid), hexa(diaminobutyric acid), hepta(diaminobutyric acid), octa(diaminobutyric acid), nona(diaminobutyric acid), deca(diaminobutyric acid), undeca(diaminobutyric acid), dodeca(diaminobutyric acid), trideca(diaminobutyric acid), tetradeca(diaminobutyric acid), pentadeca(diaminobutyric acid), hexadeca(diaminobutyric acid), heptadeca(diaminobutyric acid), octadeca(diaminobutyric acid), nonadeca(diaminobutyric acid), lysine, dilysine, trilysine, tetralysine, pentalysine, hexalysine, heptalysine, octalysine, nonalysine, decalysine, undecalysine, dodecalysine, tridecalysine, tetradecalysine, pentadecalysine, hexadecalysine, heptadecalysine, octadecalysine, nonadecalysine, oligolysines, triornithine, tetraornithine, pentaornithine, hexaornithine, heptaornithine, octaornithine, nonaornithine, decaornithine, undecaornithine, dodecaornithine, tridecaornithine, tetradecaornithine, pentadecaornithine, hexadecaornithine, heptadecaornithine, octadecaornithine, nonadecaornithine, tridiaminobutyric acid, tetradiaminobutyric acid, pentadiaminobutyric acid, hexadiaminobutyric acid, heptadiaminobutyric acid, octadiaminobutyric acid, nonadiaminobutyric acid, decadiaminobutyric acid, undecadiaminobutyric acid, dodecadiaminobutyric acid, tridecadiaminobutyric acid, tetradecadiaminobutyric acid, pentadecadiaminobutyric acid, hexadecadiaminobutyric acid, heptadecadiaminobutyric acid, octadecadiaminobutyric acid, nonadecadiaminobutyric acid,
or a polycarboxylate in bound form comprising at least 2 carboxylate groups (preferably further comprising a functional group, which is preferably an additional amino group or a carboxylic acid group, more preferably an additional carboxylic acid group),
preferably selected from di(glutamic acid), tri(glutamic acid), tetra(glutamic acid), penta(glutamic acid), hexa(glutamic acid), hepta(glutamic acid), octa(glutamic acid), nona(glutamic acid), deca(glutamic acid), undeca(glutamic acid), dodeca(glutamic acid), trideca(glutamic acid), tetradeca(glutamic acid), pentadeca(glutamic acid), hexadeca(glutamic acid), heptadeca(glutamic acid), octadeca(glutamic acid), nonadeca(glutamic acid), di(aspartic acid), tri(aspartic acid), tetra(aspartic acid), penta(aspartic acid), hexa(aspartic acid), hepta(aspartic acid), octa(aspartic acid), nona(aspartic acid), deca(aspartic acid), undeca(aspartic acid), dodeca(aspartic acid), trideca(aspartic acid), tetradeca(aspartic acid), pentadeca(aspartic acid), hexadeca(aspartic acid), heptadeca(aspartic acid), octadeca(aspartic acid), nonadeca(aspartic acid), polyethyleneimines, and polyvinylamines.

In a preferred embodiment, a moiety Hyp is selected from the group comprising, in particular consisting of, in bound form, dilysine, trilysine, tetralysine, pentalysine, hexalysine, heptalysine, octalysine, nonalysine, decalysine, undecalysine, dodecalysine, tridecalysine, tetradecalysine, pentadecalysine, hexadecalysine, heptadecalysine, octadecalysine, nonadecalysine, triornithine, tetraornithine, pentaornithine, hexaornithine, heptaornithine, octaornithine, nonaornithine, decaornithine, undecaornithine, dodecaornithine, tridecaornithine, tetradecaornithine, pentadecaornithine, hexadecaornithine, heptadecaornithine, octadecaornithine, nonadecaornithine, tridiaminobutyric acid, tetradiaminobutyric acid, pentadiaminobutyric acid, hexadiaminobutyric acid, heptadiaminobutyric acid, octadiaminobutyric acid, nonadiaminobutyric acid, decadiaminobutyric acid, undecadiaminobutyric acid, dodecadiaminobutyric acid, tridecadiaminobutyric acid, tetradecadiaminobutyric acid, pentadecadiaminobutyric acid, hexadecadiaminobutyric acid, heptadecadiaminobutyric acid, octadecadiaminobutyric acid, nonadecadiaminobutyric acid, di(glutamic acid), tri(glutamic acid), tetra(glutamic acid), penta(glutamic acid), hexa(glutamic acid), hepta(glutamic acid), octa(glutamic acid), nona(glutamic acid), deca(glutamic acid), undeca(glutamic acid), dodeca(glutamic acid), trideca(glutamic acid), tetradeca(glutamic acid), pentadeca(glutamic acid), hexadeca(glutamic acid), heptadeca(glutamic acid), octadeca(glutamic acid), nonadeca(glutamic acid), di(aspartic acid), tri(aspartic acid), tetra(aspartic acid), penta(aspartic acid), hexa(aspartic acid), hepta(aspartic acid), octa(aspartic acid), nona(aspartic acid), deca(aspartic acid), undeca(aspartic acid), dodeca(aspartic acid), trideca(aspartic acid), tetradeca(aspartic acid), pentadeca(aspartic acid), hexadeca(aspartic acid), heptadeca(aspartic acid), octadeca(aspartic acid), nonadeca(aspartic acid), polyethyleneimines, and low-molecular weight PEI.

More preferably, a moiety Hyp is selected from the group comprising, more preferably consisting of, in bound form, trilysine, tetralysine, pentalysine, hexalysine, heptalysine, octalysine, nonalysine, decalysine, undecalysine, dodecalysine, tridecalysine, tetradecalysine, pentadecalysine, hexadecalysine, and heptadecalysine, even more preferably a moiety Hyp of formula (I) comprises, preferably consists of, in bound form, trilysine, heptalysine or pentadecalysine.

In a preferred embodiment, Hyp has a molecular weight of from 0.1 kDa to 4 kDa, in particular of from 0.2 kDa to 2 kDa.

According to formula (I), a moiety Hyp of formula (I) is connected to m moieties L, either directly (if x of formula (I) is 0) or indirectly through SP (if x of formula (I) is 1). It is understood that each linkage between a moiety Hyp and a moiety L of formula (I) may independently be direct or indirect through a moiety SP. Preferably, all linkages between a moiety Hyp and a moiety L of formula (I) are either direct or indirect through a moiety SP.

In a preferred embodiment, a moiety Hyp of formula (I) is connected to a moiety SP (if x of formula (I) is 1) or to a moiety L (if x of formula (I) is 0) through a linkage group selected from amide, carbamate, ester, ether, amine or thioether; preferably, a moiety Hyp of formula (I) is connected to a moiety SP (if x of formula (I) is 1) or to a moiety L (if x of formula (I) is 0) through a linkage group selected from amide, thioether or ether, even more preferably through an amide group.

Optionally, a functional group of Hyp which is not connected to a moiety SP or a moiety L of formula (I) may be capped with a suitable capping reagent or may optionally be connected to at least one targeting moiety, in particular through permanent linkages. Preferably, all functional groups of a moiety Hyp of formula (I) are connected to a moiety L or SP. Targeting moieties, if present, may be conjugated to Hyp either directly or indirectly through spacer moieties.

Examples of suitable capping moieties are linear, branched or cyclic C₁₋₈ alkyl groups.

In one embodiment, each moiety Hyp of formula (I) is directly or indirectly connected to at least two moieties L, such as to at least three moieties L, to at least four moieties L or to at least five moieties L.

In a further preferred embodiment, each branched moiety Hyp has at least 1 branching and is conjugated to at least 2 moieties L (either directly or indirectly) and has at most 63 branchings and is at most conjugated to 64 moieties L (either directly or indirectly). More preferably each branched moiety Hyp has at least 1 branching and is conjugated to at least 2 moieties L (either directly or indirectly) and has at most 31 branchings and is at most conjugated to 32 moieties L (either directly or indirectly).

A moiety SP of formula (I) is a spacer moiety connecting a moiety Hyp to a moiety L of formula (I).

Preferably, SP is selected from COOR¹; OR¹; C(O)R¹; C(O)N(R¹R^{1a}); S(O)₂N(R¹R^{1a}); S(O)N(R¹R^{1a}); S(O)₂R¹; S(O)R¹; N(R¹)S(O)₂N(R^{1a}R^{1b}); SR¹; N(R¹R^{1a}); OC(O)R¹; N(R¹)C(O)R^{1a}; N(R¹)S(O)₂R^{1a}; N(R¹)S(O)R^{1a}; N(R¹)C(O)OR^{1a}; N(R¹)C(O)N(R^{1a}R^{1b}); OC(O)N(R¹R^{1a}); T; C₁₋₅₀ alkyl; C₂₋₅₀ alkenyl; and C₂₋₅₀ alkynyl,
wherein T, C₁₋₅₀ alkyl, C₂₋₅₀ alkenyl, and C₂₋₅₀ alkynyl are optionally substituted with one or more R², which are the same or different,
and wherein C₁₋₅₀ alkyl; C₂₋₅₀ alkenyl; and C₂₋₅₀ alkynyl are optionally interrupted by one or more groups selected from the group consisting of -T-, —C(O)O—; —O—; —C(O)—; —C(O)N(R³)—; —S(O)₂N(R³)—; —S(O)N(R³)—; —S(O)₂—; —S(O)—; —N(R³)S(O)₂N(R^{3a})—; —S—; —N(R³)—; —OC(O)R^{3a}; —N(R³)C(O)—; —N(R³)S(O)₂—; —N(R³)S(O)—; —N(R³)C(O)O—; —N(R³)C(O)N(R^{3a})—; and —OC(O)N(R³R^{3a});
R¹, R^{1a}, R^{1b} are independently selected from the group consisting of H; T; and C₁₋₅₀ alkyl; C₂₋₅₀ alkenyl; and C₂₋₅₀ alkynyl,
wherein T, C₁₋₅₀ alkyl, C₂₋₅₀ alkenyl, and C₂₋₅₀ alkynyl are optionally substituted with one or more R², which are the same or different,
and wherein C₁₋₅₀ alkyl; C₂₋₅₀ alkenyl; and C₂₋₅₀ alkynyl are optionally interrupted by one or more groups selected from the group consisting of T, —C(O)O—; —O—; —C(O)—; —C(O)N(R³)—; —S(O)₂N(R³)—; —S(O)N(R³)—; —S(O)₂—; —S(O)—; —N(R³)S(O)₂N(R^{3a})—; —S—; —N(R³)—; —OC(O)R³; —N(R³)C(O)—; —N(R³)S(O)₂—; —N(R³)S(O)—; —N(R³)C(O)O—; —N(R³)C(O)N(R^{3a}); and —OC(O)N(R³R^{3a});
T is selected from the group consisting of phenyl; naphthyl; indenyl; indanyl; tetralinyl; C₃₋₁₀ cycloalkyl; 4- to 7-membered heterocyclyl; or 9- to 11-membered heterobicyclyl, wherein T is optionally substituted with one or more R², which are the same or different;
R² is halogen; CN; oxo (═O); COOR⁴; OR⁴; C(O)R⁴; C(O)N(R⁴R^{4a}); S(O)₂N(R⁴R^{4a}); S(O)N(R⁴R^{4a}); S(O)₂R⁴; S(O)R⁴; N(R⁴)S(O)₂N(R^{4a}R^{4b}); SR⁴; N(R⁴R^{4a}); NO₂; OC(O)R⁴; N(R⁴)C(O)R^{4a}; N(R⁴)S(O)₂R^{4a}; N(R⁴)S(O)R^{4a}; N(R⁴)C(O)OR^{4a}; N(R⁴)C(O)N(R^{4a}R^{4b}); OC(O)N(R⁴R^{4a}); or C₁₋₆ alkyl, wherein C₁₋₆ alkyl is optionally substituted with one or more halogen, which are the same or different;
R³, R^{3a}, R⁴, R^{4a}, R^{4b} are independently selected from the group consisting of H; and C₁₋₆ alkyl, wherein C₁₋₆ alkyl is optionally substituted with one or more halogen, which are the same or different.

A moiety L of formula (I) may be chosen depending on the one or more functional groups present in the corresponding drug of a biologically active moiety D of formula (I). Suitable moieties L are known to the person skilled in the art and examples are given in the following sections.

In a preferred embodiment, a moiety L of formula (I) is a traceless prodrug linker. Preferably, all moieties L of formula (I) are traceless prodrug linkers.

A preferred reversible prodrug linker moiety for amine-comprising drugs is described in WO-A 2005/099768. Therefore, the following sub-structures selected from the general formulae (II) and (III) are preferred embodiments for —(SP)ₓ-L-D for the water-soluble carrier-linked prodrug of the present invention according to formula (I):

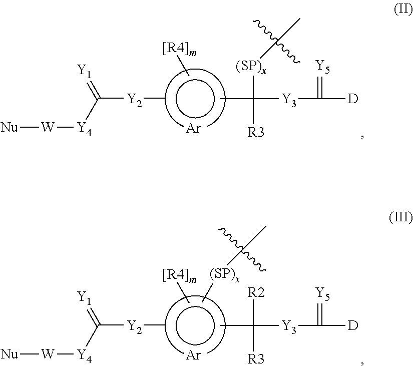

wherein the dashed line indicates attachment to a moiety Hyp of formula (I), which moiety Hyp of formula (I) is connected to m sub-structures of formula(s) (II) and/or (III), and
SP, x, Y₁, Y₂, Y₃, Y₄, Y₅, R2, R3, R4, Nu, W, m, and D of formulas (II) and (III) have the following meaning:
D is an amine-comprising biologically active moiety D of formula (I) which is attached to the rest of the sub-structure shown in formula (II) or (III) by forming a —O—(C═O)—N—; —O—(C═S)—N—; —S—(C═O)—N—; or —S—(C═S)—N— linkage,
SP is the spacer moiety SP of formula (I),
x is 0 or 1,
Y₁ and Y₂ are each independently O, S or NR6,
Y₃ is O or S,
Y₄ is O, NR6, or —C(R7)(R8)-,
Y₅ is O or S,
each of R2 and R3 is a moiety selected from the group consisting of hydrogen, substituted or unsubstituted linear, branched or cyclical alkyl or heteroalkyl groups, aryls, substituted aryls, substituted or unsubstituted heteroaryls, cyano groups, nitro groups, halogens, carboxy groups, carboxyalkyl groups, alkylcarbonyl groups and carboxamidoalkyl groups,
R4 is selected from the group consisting of hydrogen, substituted or unsubstituted linear, branched or cyclical alkyls or heteroalkyls, aryls, substituted aryls, substituted or unsubstituted heteroaryl, substituted or unsubstituted linear, branched or cyclical alkoxys, substituted or unsubstituted linear, branched or cyclical heteroalkyloxys, aryloxys or heteroaryloxys, cyano groups and halogens,
R6 is selected from hydrogen, substituted or unsubstituted linear, branched or cyclical alkyls or heteroalkyls, aryls, substituted aryls and substituted or unsubstituted heteroaryls,
R7 and R8 are each independently selected from the group consisting of hydrogen, substituted or unsubstituted linear, branched or cyclical alkyls or heteroalkyls, aryls, substituted aryls, substituted or unsubstituted heteroaryls, carboxyalkyl groups, alkylcarbonyl groups, carboxamidoalkyl groups, cyano groups, and halogens,
W is selected from substituted or unsubstituted linear, branched or cyclical alkyls, aryls, substituted aryls, substituted or unsubstituted linear, branched or cyclical heteroalkyls, substituted or unsubstituted heteroaryls,
Nu is a nucleophile,
m is zero or a positive integer, and
Ar is a multi-substituted aromatic hydrocarbon or multi-substituted aromatic heterocycle.

Another suitable reversible prodrug linker moiety for amine-comprising drugs is described in WO-A 2006/136586. Accordingly, the following sub-structures selected from the general formulas (IV), (V) and (VI) are preferred embodiments for —(SP)ₓ-L-D for the water-soluble carrier-linked prodrug of the present invention according to formula (I):

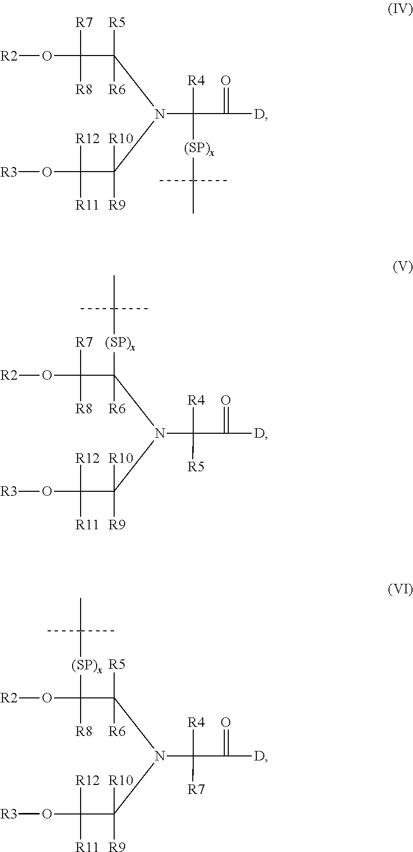

wherein the dashed line indicates attachment to a moiety Hyp of formula (I), which moiety Hyp of formula (I) is connected to m sub-structures of formula(s) (IV), (V) and/or (VI), and
wherein SP, x, R2, R3, R4, R5, R6, R7, R8, R9, R10, R11, R12 and D of formulas (IV), (V) and (VI) have the following meaning:
D is an amine-comprising biologically active moiety D of formula (I),
SP is the spacer moiety SP of formula (I),
x is 0 or 1,
Y1 is O, S, NR6, succinimide, maleimide, an unsaturated carbon-carbon bond, or any heteroatom-containing a free electron pair or Y1 is absent,
R2 and R3 are selected independently from hydrogen, acyl groups, and protecting groups for hydroxyl groups;
R4 to R12 are selected independently from hydrogen, substituted or non-substituted linear, branched or cyclical alkyl or heteroalkyl, aryls, substituted aryls, substituted or non-substituted heteroaryls, cyano, nitro, halogen, carboxy, and carboxamide.

Another suitable reversible prodrug linker moiety for primary amine- or secondary amine-comprising drugs is described in WO-A 2009/095479. Accordingly, the following sub-structure of the general formula (VII) is a preferred embodiment for —(SPX)ₓ-L-D for the water-soluble carrier-linked prodrug of the present invention according to formula (I):

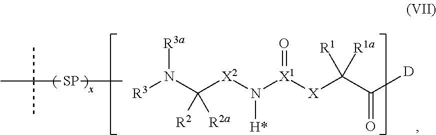

wherein the dashed line indicates attachment to a moiety Hyp of formula (I), which moiety Hyp of formula (I) is connected to m sub-structures of formula (VII);
the moiety [symbol] is attached to any one of R¹, R^{1a}, R², R^{2a}, R³, R^{3a}, X, and X²; and
wherein SP, x, D, X, X¹, X², R¹, R^{1a}, R², R^{2a}, R³, and R^{3a} of formula (VII) have the following meaning:
D is a primary amine- or secondary amine-comprising biologically active moiety D;
SP is the spacer moiety SP of formula (I);
x is 0 or 1:
X is C(R⁴R^{4a}); N(R⁴); O; C(R⁴R^{4a})—C(R⁵R^{5a}); C(R⁵R^{5a})—C(R⁴R^{4a}); C(R⁴R^{4a})—N(R); N(R⁶)—C(R⁴R^{4a}); C(R⁴R^{4a})—O; or O—C(R⁴R^{4a});
X¹ is C; or S(O);
X² is C(R⁷, R^{7a}); or C(R⁷, R^{7a})—C(R⁸, R^{8a});
R¹, R^{1a}, R², R^{2a}, R³, R^{3a}, R⁴, R^{4a}, R⁵, R^{5a}, R⁶, R⁷, R^{7a}, R⁸, R^{8a} are independently selected from the group consisting of H; and C₁₋₄ alkyl;
optionally, one or more of the pairs R^{1a}/R^{4a}, R^{1a}/R^{5a}, R^{4a}/R^{5a}, R^{4a}/R^{5a}, R^{7a}/R^{8a} form a chemical bond;
optionally, one or more of the pairs R¹/R^{1a}, R²/R^{2a}, R⁴/R^{4a}, R⁵/R^{5a}, R⁷/R^{7a}, R⁸/R^{8a} are joined together with the atom to which they are attached to form a C₃₋₇ cycloalkyl or 4- to 7-membered heterocyclyl;
optionally, one or more of the pairs R¹/R⁴, R¹/R⁵, R¹/R⁶, R⁴/R⁵, R⁷/R⁸, R²/R³ are joined together with the atoms to which they are attached to form a ring A;
optionally, R³/R^{3a} are joined together with the nitrogen atom to which they are attached to form a 4- to 7-membered heterocycle;
A is selected from the group consisting of phenyl, naphthyl, indenyl, indanyl, tetralinyl, C₃₋₁₀ cycloalkyl, 4- to 7-membered heterocyclyl, and 9- to 11-membered heterobicyclyl.

In the sub-structure —(SP)ₓ-L-D of formula (VII) the moiety L is of formula (VIIa):

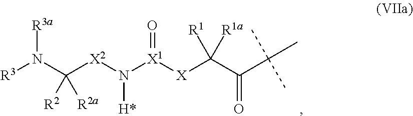

wherein
the dashed line indicates attachment to D of formula (VII), and
X, X¹, X², R¹, R^{1a}, R², R^{2a}, R³, and R^{3a} of formula (VIIa) are defined as in formula (VII).

Optionally, L in formula (VII) is further substituted, provided that the hydrogen marked with the asterisk in formula (VII) is not replaced by a substituent. Preferably, the one or more further optional substituents are independently selected from the group consisting of halogen, CN, COOR⁹, OR⁹, C(O)R⁹, C(O)N(R⁹R^{9a}), S(O)₂N(R⁹R^{9a}), S(O)N(R⁹R^{9a}), S(O)₂R⁹, S(O)R⁹, N(R⁹)S(O)₂N(R^{9a}R^{9b}), SR⁹, N(R⁹R^{9a}), NO₂, OC(O)R⁹, N(R⁹)C(O)R^{9a}, N(R⁹)S(O)₂R^{9a}, N(R⁹)S(O)R^{9a}, N(R⁹)C(O)OR^{9a}, N(R⁹)C(O)N(R^{9a}R^{9b}), OC(O)N(R⁹R^{9a}), T, C₁₋₅₀ alkyl, C₂₋₅₀ alkenyl, and C₂₋₅₀ alkynyl,
wherein T, C₁₋₅₀ alkyl, C₂₋₅₀ alkenyl, and C₂₋₅₀ alkynyl are optionally substituted with one or more R¹⁰, which are the same or different, and wherein C₁₋₅₀ alkyl; C₂₋₅₀ alkenyl; and C₂₋₅₀ alkynyl are optionally interrupted by one or more groups selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹¹R^{11a});
R⁹, R^{9a}, R^{9b} are independently selected from the group consisting of H; T; and C₁₋₅₀ alkyl; C₂₋₅₀ alkenyl; and C₂₋₅₀ alkynyl,
   wherein T, C₁₋₅₀ alkyl, C₂₋₅₀ alkenyl, and C₂₋₅₀ alkynyl are optionally substituted with one or more R¹⁰, which are the same or different, and wherein C₁₋₅₀ alkyl; C₂₋₅₀ alkenyl; and C₂₋₅₀ alkynyl are optionally interrupted by one or more groups selected from the group consisting of T, —C(O)O—, —O—, —C(O)—, —C(O)N(R¹¹)—, —S(O)₂N(R¹¹)—, —S(O)N(R¹¹)—, —S(O)₂—, —S(O)—, —N(R¹¹)S(O)₂N(R¹¹)—, —S—, —N(R¹¹)—, —OC(O)R¹¹, —N(R¹¹)C(O)—, —N(R¹¹)S(O)₂—, —N(R¹¹)S(O)—, —N(R¹¹)C(O)O—, —N(R¹¹)C(O)N(R^{11a})—, and —OC(O)N(R¹¹R^{11a}),
   T is selected from the group consisting of phenyl, naphthyl, indenyl, indanyl, tetralinyl, C₃₋₁₀ cycloalkyl, 4- to 7-membered heterocyclyl, and 9- to 11-membered heterobicyclyl, wherein T is optionally substituted with one or more R¹⁰, which are the same or different,
   R¹⁰ is halogen, CN, oxo (═O), COOR¹², OR¹², C(O)R¹², C(O)N(R¹²R^{12a}), S(O)₂N(R¹²R^{12a}), S(O)N(R¹²R^{12a}), S(O)₂R¹², S(O)R¹², N(R¹²)S(O)₂N(R^{12a}R^{12b}), SR¹², N(R¹²R^{12a}), NO₂, OC(O)R¹², N(R¹²)C(O)R^{12a}, N(R¹²)S(O)₂R^{12a}, N(R¹²)S(O)R^{12a}, N(R¹²)C(O)OR^{12a}, N(R¹²)C(O)N(R^{12a}R^{12b}), OC(O)N(R²R^{12a}), or C₁₋₆ alkyl, wherein C₁₋₆ alkyl is optionally substituted with one or more halogen, which are the same or different,
   R¹¹, R^{11a}, R¹², R^{12a}, R^{12b} are independently selected from the group consisting of H; or C₁₋₆ alkyl, wherein C₁₋₆ alkyl is optionally substituted with one or more halogen, which are the same or different.

The term “interrupted” means that between two carbons a group is inserted or at the end of the carbon chain between the carbon and hydrogen.

Preferred moieties L according to formula (VII) are selected from the group consisting of:

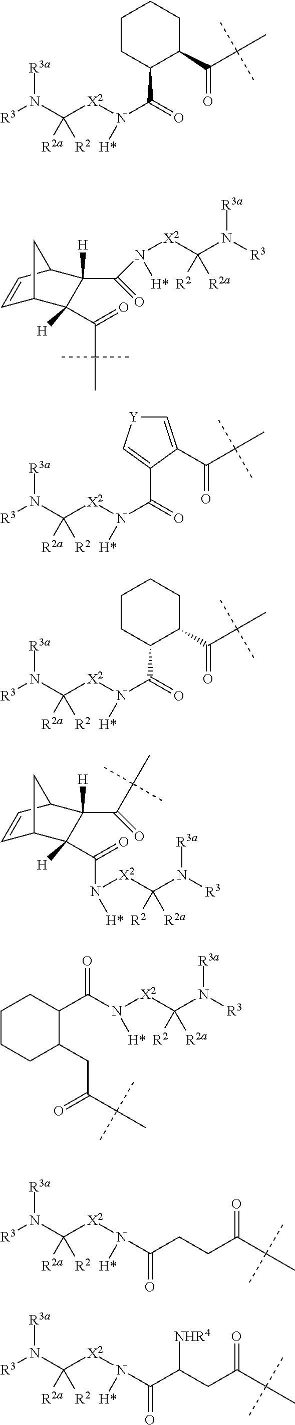

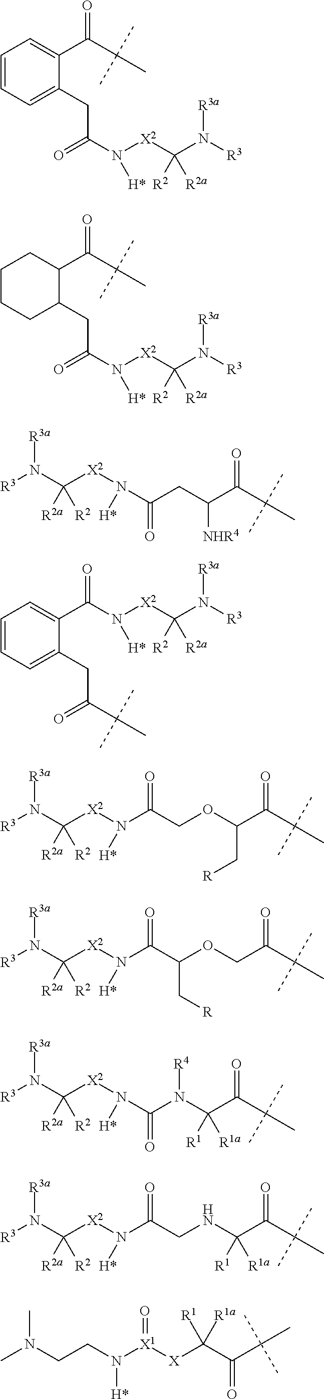

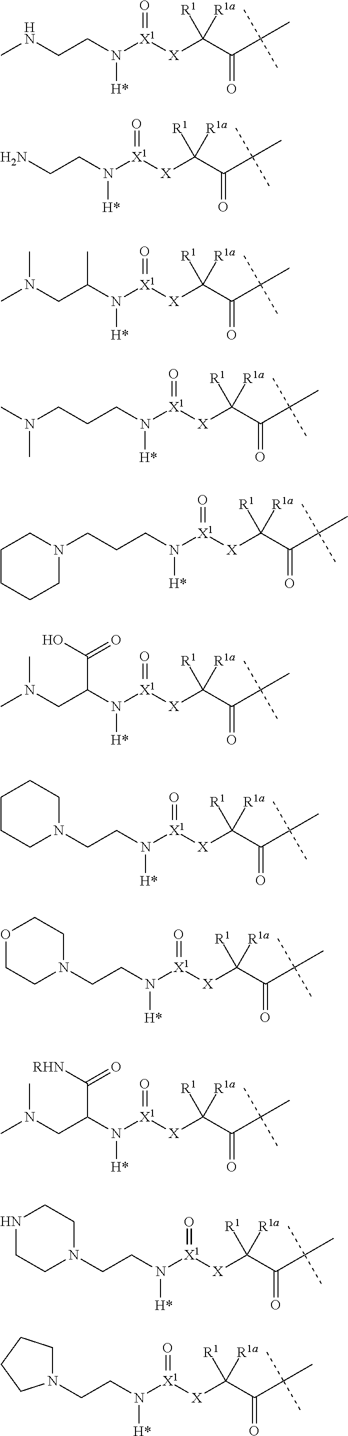

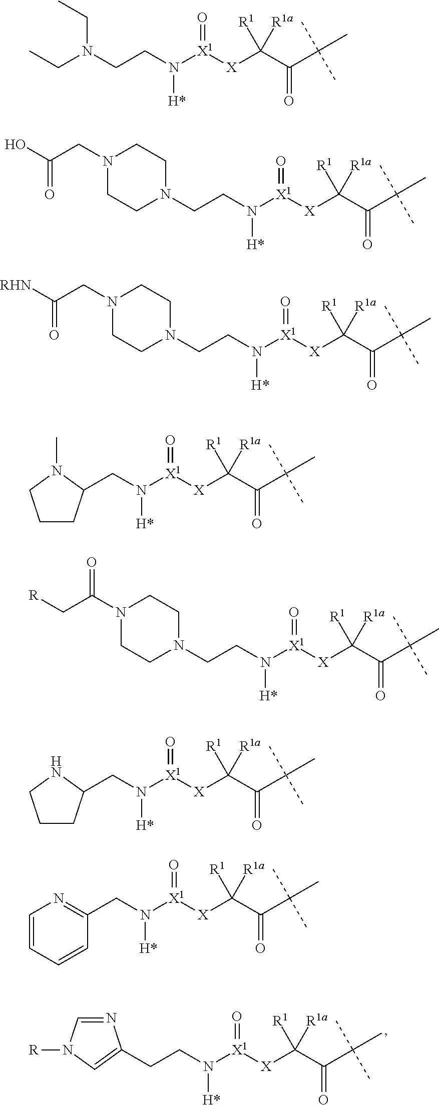

wherein
dashed lines indicate attachment to D of formula (VII),
R is H or C₁₋₄ alkyl,
Y is NH, O or S,
and R¹, R^{1a}, R², R^{2a}, R³, R^{3a}, R⁴, X, X¹, X² have the meaning as indicated in formula (VII).

Even more preferred moieties L according to formula (VII) are selected from the group consisting of:

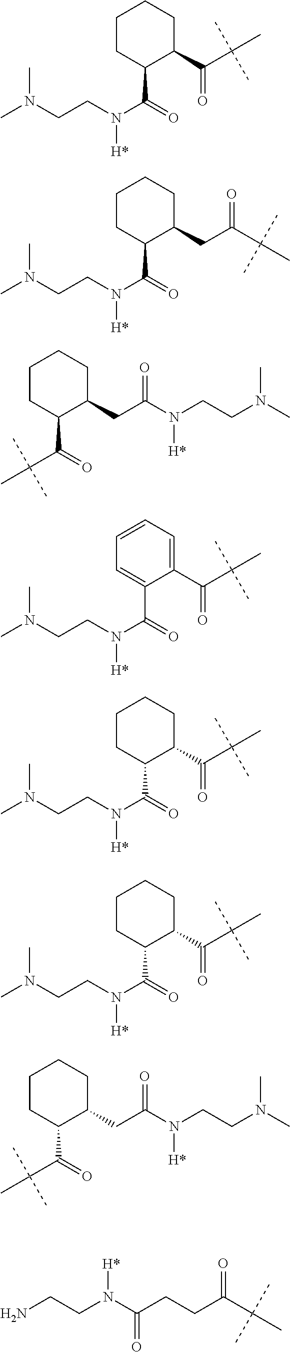

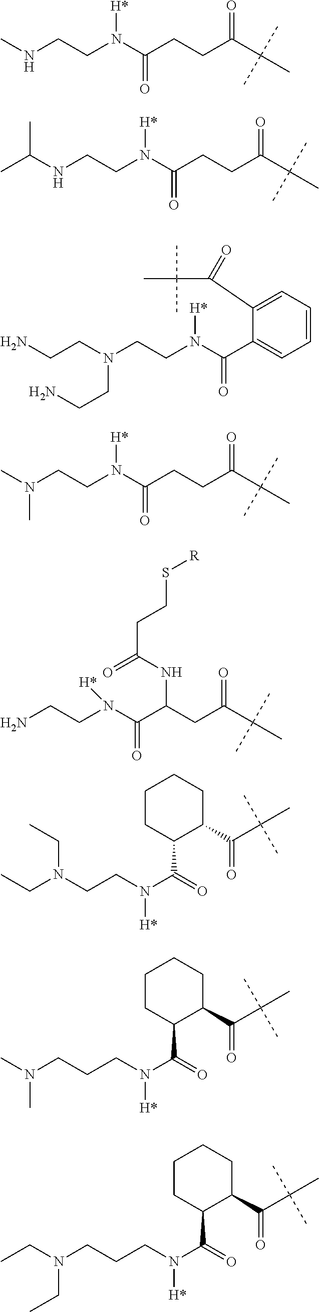

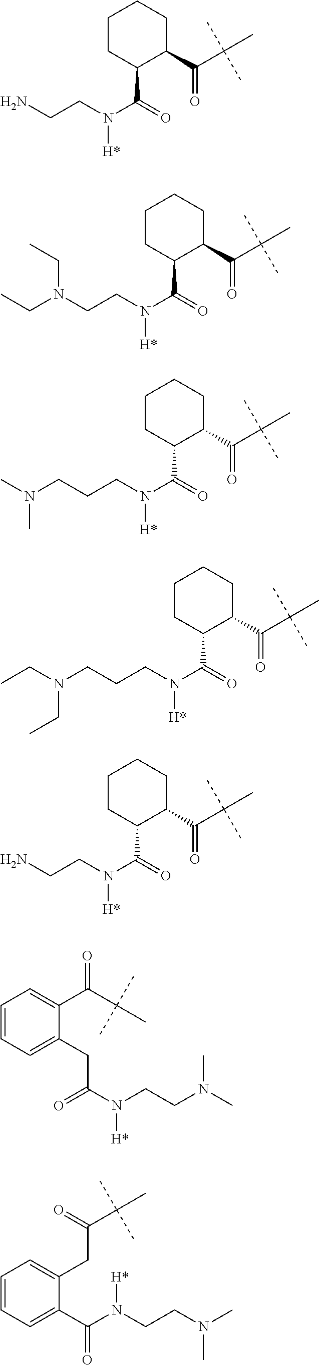

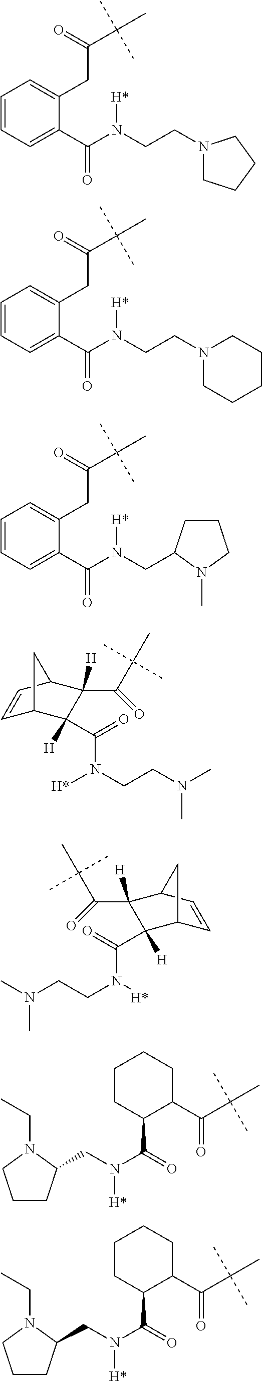

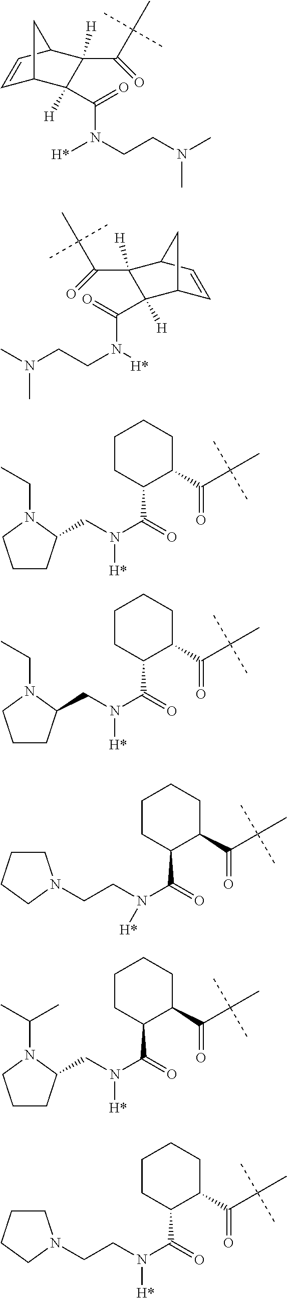

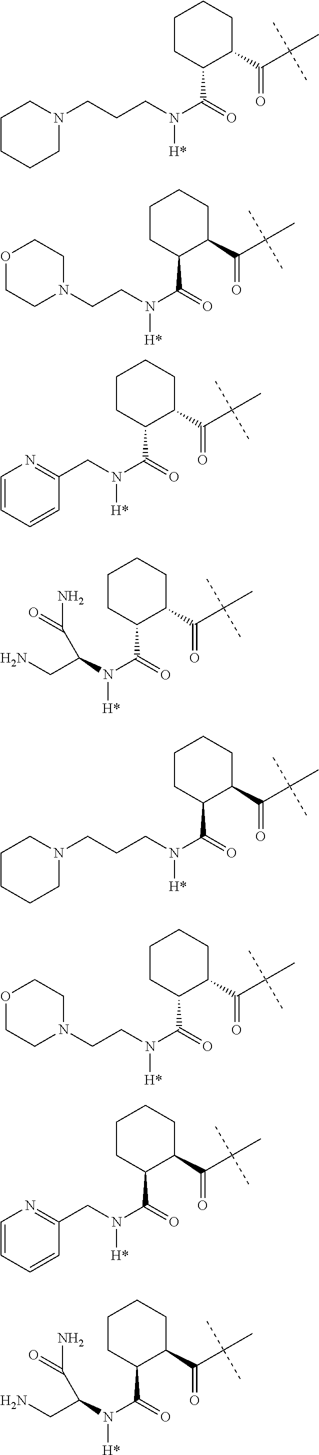

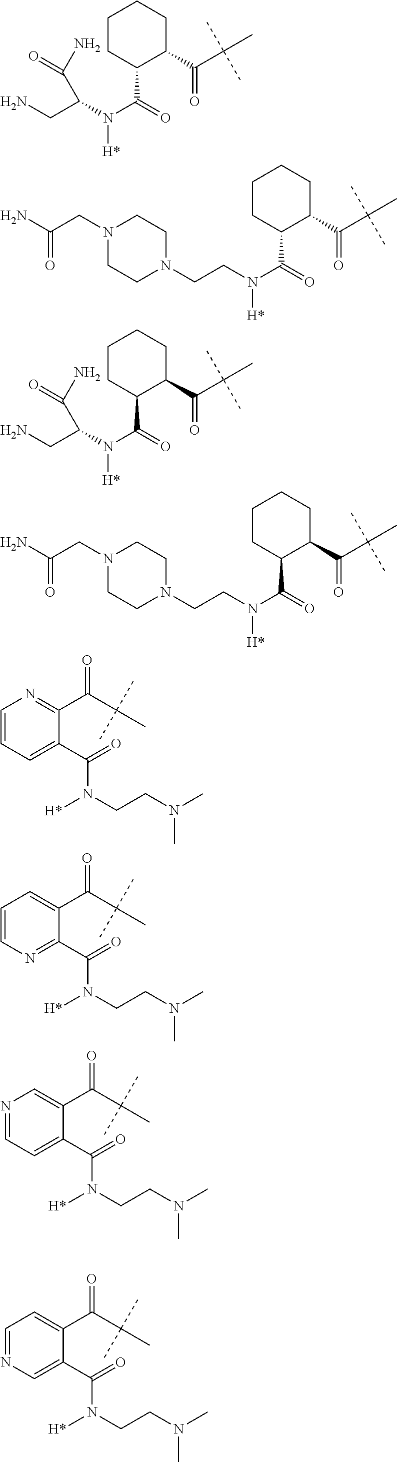

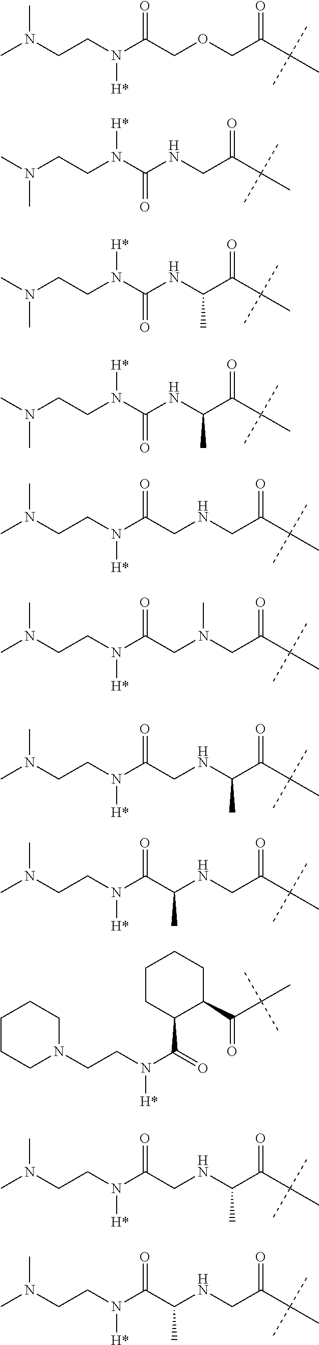

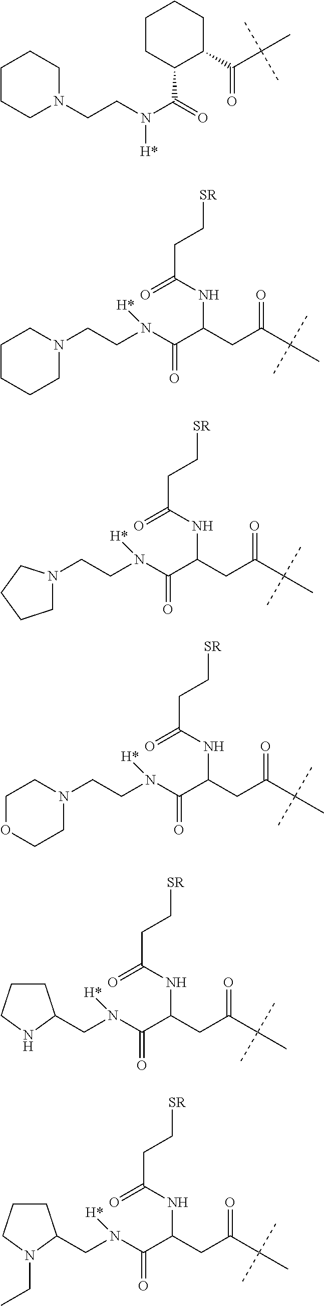

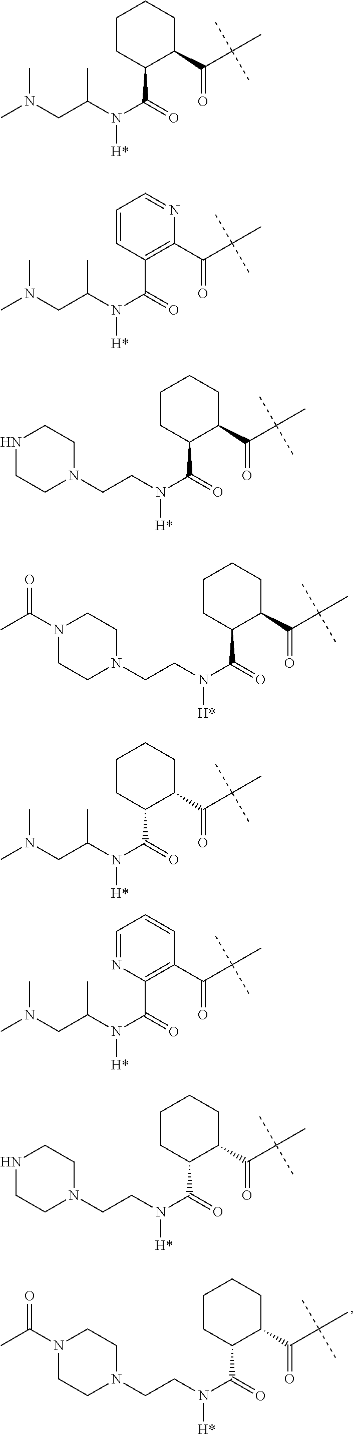

wherein
dashed lines indicate attachment to D of formula (VII), and
R is H or C₁₋₄ alkyl

In yet another preferred embodiment the sub-structure —(SP)ₓ-L-D of formula (I) for the water-soluble carrier-linked prodrug of the present invention is of formula (VIII):

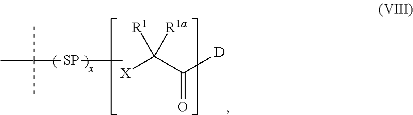

wherein the dashed line indicates attachment to a moiety Hyp of formula (I), which moiety Hyp of formula (I) is connected to m sub-structures of formula (VIII),
the moiety [symbol] is attached to any one of R¹, R^{1a}, and X; and
wherein SP, x, D, X, R¹, and R^{1a} of formula (VIII) have the following meaning:
D is a primary amine- or secondary amine-comprising biologically active moiety D,
SP is the spacer moiety SP of formula (I);
x is 0 or 1:
X is H or C₁₋₅₀ alkyl, optionally interrupted by one or more groups selected from —NH—, —C(C₁₋₄ alkyl)—, —O—, —C(O)— or —C(O)NH—,
R¹ and R^{1a} are independently selected from the group consisting of H and C₁-C₄ alkyl,

Optionally, the sub-structure of formula (VIII) is further substituted.

In the sub-structure —(SP)ₓ-L-D of formula (VIII) the moiety L is of formula (VIIIa):

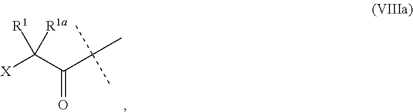

wherein
the dashed line indicates attachment to D of formula (VIII) and
X, R¹ and R^{1a} of formula (VIIIa) are defined as in formula (VIII).

More preferably, L of the sub-structure of formula (VIII) comprises one of the fragments of formulas (VIIIb) or (VIIIc), wherein the dashed line marked with an asterisk indicates attachment to D by forming an amide bond with the aromatic amino group of D and the unmarked dashed line indicates attachment to the rest of L of formula (VIII) and wherein the structures of formulas (VIIIb) and (VIIIc) are optionally further substituted:

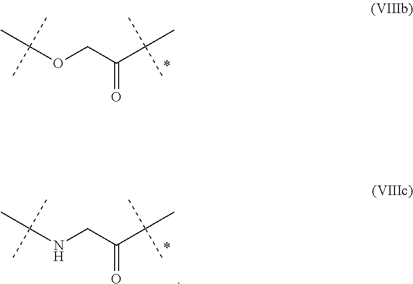

More preferably, L of the sub-structure of formula (VIII) comprises one of the fragments of formulas (VIIIba), (VIIIca), or (VIIIcb), wherein the dashed line marked with an asterisk indicates attachment to D of formula (VIII) by forming an amide bond with the aromatic amino group of D and the unmarked dashed line indicates attachment to the rest of L of formula (VIII):

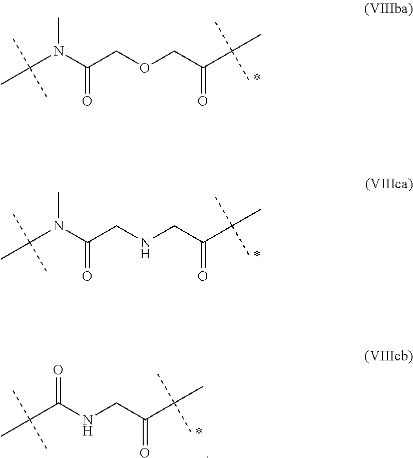

Another preferred reversible prodrug linker moiety L for aromatic amine-comprising drugs is described in WO 2011/012721. Therefore, the following sub-structure of the general formula (IX) is a preferred embodiment for —(SP)ₓ-L-D for the water-soluble carrier-linked prodrug of the present invention according to formula (I):

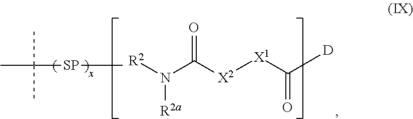

wherein the dashed line indicates attachment to a moiety Hyp of formula (I), which moiety Hyp of formula (I) is connected to m sub-structures of formula (IX),
D is connected to the rest of the sub-structure of formula (IX) through an aromatic amine group of D by forming an amide bond,
the moiety [symbol] is attached to any one of R², R^{2a}, X¹, and X²; and
wherein D, SP, x, X¹, X², R², and R^{2a} in formula (IX) have the following meaning:
D is an aromatic amine-comprising biologically active moiety D,
SP is the spacer moiety SP of formula (I),
x is 0 or 1,
X¹ is C(R¹R^{1a}) or a cyclic fragment selected from C₃₋₇ cycloalkyl, 4- to 7-membered heterocyclyl, phenyl, naphthyl, indenyl, indanyl, tetralinyl, and 9- to 11-membered heterobicyclyl,
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, and O—C(R³R^{3a}),
wherein in case X¹ is a cyclic fragment, X² is a chemical bond, C(R³R^{3a}), N(R³) or O,
optionally, in case X¹ is a cyclic fragment and X² is C(R³R^{3a}), the order of the X¹ fragment and the X² fragment within the sub-structure —(SP)ₓ-L-D shown in formula (IX) may be changed,
R¹, R³ and R⁴ are independently selected from the group consisting of H, C₁₋₄ alkyl and —N(R⁵R^{5A}),
R^{1a}, R², R^{2a}, R^{3a}, R^{4a} and R^{5a} are independently selected from the group consisting of H, and C₁₋₄ alkyl,
optionally, one of the pairs R^{2a}/R², R^{2a}/R^{3a}, R^{2a}/R^{4a} are joined to form a 4- to 7-membered at least partially saturated heterocycle,
R⁵ is C(O)R⁶,
R⁶ is C₁₋₄ alkyl, and
optionally, one of the pairs R^{1a}/R^{4a}, R^{3a}/R^{4a} or R^{1a}/R^{3a} form a chemical bond.

Optionally, the sub-structure —(SP)ₓ-L-D of formula (IX) is further substituted.

In the sub-structure —(SP)ₓ-L-D of formula (IX) the moiety L is of formula (IXa):

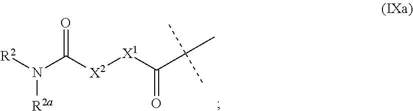

wherein
the dashed line indicates attachment to D of formula (IX), and
X¹, X², R², and R^{2a} of formula (IXa) are used as defined in formula (IX).

More preferably, the moiety L according to formula (IX) is selected from the following formulas:

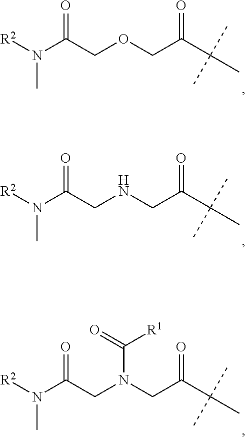

wherein the dashed line indicates attachment to D of formula (IX), and
R¹ and R² are used as defined in formula (IX).

Preferably, in formula (IX) R^{1a}, R², R^{2a}, R^{3a}, R^{4a} and R^{5a} are independently selected from the group consisting of H, and C₁₋₄ alkyl.

Another preferred reversible prodrug linker moiety L for aromatic amine-comprising drugs is described in WO 2011/012722. Therefore, the following sub-structure of the general formula (X) is a preferred embodiment for —(SP)ₓ-L-D for the water-soluble carrier-linked prodrug of the present invention according to formula (I):

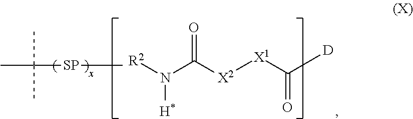

wherein the dashed line indicates attachment to a moiety Hyp of formula (I), which moiety Hyp of formula (I) is connected to m sub-structures of formula (X),
D is connected through an aromatic amine group of D to the rest of the sub-structure of formula (X) by forming an amide bond,
the moiety [symbol] is attached to any one of R², X¹, and X²; and
wherein D, SP, x, X¹, X², R², and R^{2a} in formula (X) have the following meaning:
D is an aromatic amine-comprising biologically active moiety D,
SP is the spacer moiety SP of formula (I),
x is 0 or 1,
X¹ is C(R¹R^{1a}) or a cyclic fragment selected from C₃₋₇ cycloalkyl, 4 to 7 membered heterocyclyl, phenyl, naphthyl, indenyl, indanyl, tetralinyl, and 9 to 11 membered heterobicyclyl,
wherein in case X¹ is a cyclic fragment, said cyclic fragment is incorporated into —(SP)ₓ-L-D of formula (X) via two adjacent ring atoms and the ring atom of X¹, which is adjacent to the carbon atom of the amide bond, is also a carbon atom,
X² is a chemical bond or selected from C(R³R^{3a}), N(R³), O, C(R³R³)—C(R⁴R^{4a}), C(R³R^{3a})—N(R⁴), N(R³)—C(R⁴R^{4a}), C(R³R^{3a})—O, and O—C(R³R^{3a}),
wherein in case X¹ is a cyclic fragment, X² is a chemical bond, C(R³R^{3a}), N(R³) or O,
optionally, in case X¹ is a cyclic fragment and X² is C(R³R^{3a}), the order of the X¹ fragment and the X² fragment within the sub-structure —(SP)ₓ-L-D shown in formula (X) may be changed and the cyclic fragment is incorporated into the sub-structure —(SP)ₓ-L-D of formula (X) via two adjacent ring atoms,
R¹, R³ and R⁴ are independently selected from the group consisting of H, C₁₋₄ alkyl and —N(R⁵R^{5a}),
R^{1a}, R², R^{3a}, R^{4a} and R^{5a} are independently selected from the group consisting of H, and C₁₋₄ alkyl,
R⁵ is C(O)R⁶,
R⁶ is C₁₋₄ alkyl,
optionally, one of the pairs R^{1a}/R^{4a}, R^{3a}/R^{4a} or R^{1a}/R^{3a} form a chemical bond, provided that the hydrogen marked with the asterisk in formula (X) is not replaced by the moiety [symbol] of formula (X).

In the sub-structure —(SP)ₓ-L-D of formula (X) the moiety L is of formula (Xa):

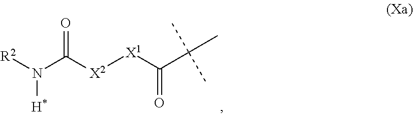

wherein
the dashed line indicates attachment to D of formula (X), and
X¹, X², and R² of formula (Xa) are used as defined in formula (X).

Optionally, the moiety L of formula (X) is further substituted.

More preferably, the moiety L according to formula (X) is selected from the group consisting of formulas (i) through (xxix):

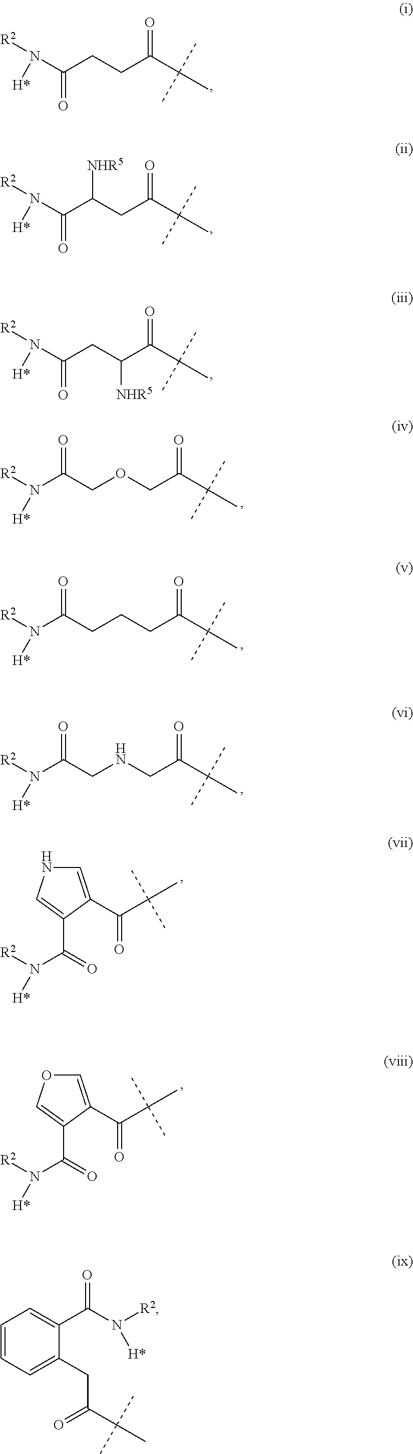

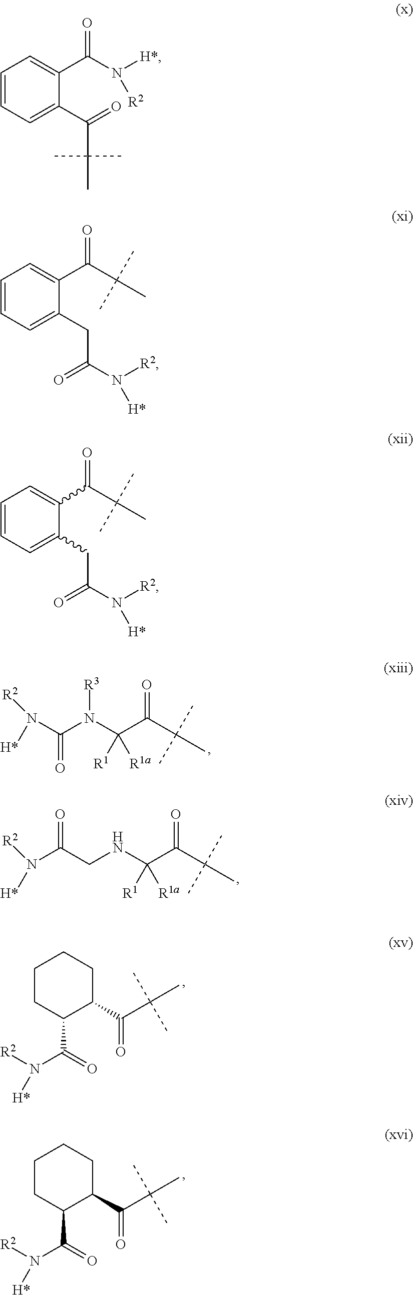

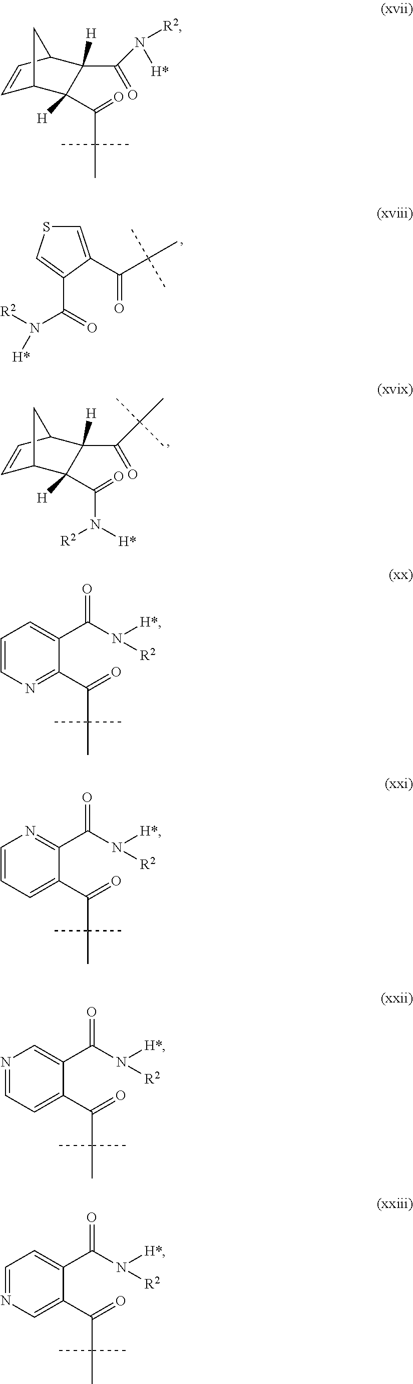

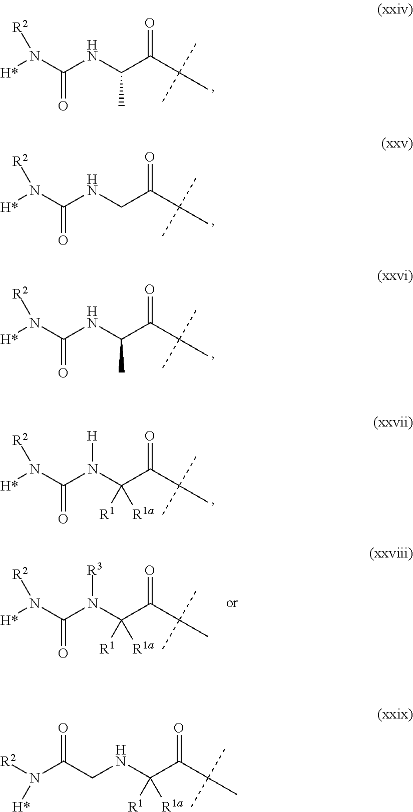

wherein the dashed line indicates attachment to D, and
R¹, R^{1a}, R², R³, and R⁵ are used as defined in formula (X).

The amino substituent of the aromatic fragment of D forms together with the carbonyl-fragment (—C(O)—) on the right hand side of L (as depicted in formula (X)) an amide bond between L and D. By consequence, D and L of formula (X) are connected (chemically bound) by an amide fragment of the general structure Y¹—C(O)—N(R)—Y². Y¹ indicates the remaining parts of the sub-structure of formula (X) and Y² indicates the aromatic fragment of D. R is a substituent, such as C₁₋₄ alkyl or preferably hydrogen.

As indicated above, X¹ of formula (X) may also be a cyclic fragment such as C₃₋₇ cycloalkyl, phenyl or indanyl. In case X¹ is such a cyclic fragment, the respective cyclic fragment is incorporated into L of formula (X) via two adjacent ring atoms (of said cyclic fragment). For example, if X¹ is phenyl, the phenyl fragment of L is bound to X² of L via a first (phenyl) ring atom being in α-position (adjacent) to a second (phenyl) ring atom, which itself is bound to the carbon atom of the carbonyl-fragment on the right hand side of L according to formula (X), i.e. the carbonyl fragment which together with the aromatic amino group of D forms an amide bond.

Preferably, L of formula (X) is defined as follows:
X¹ is C(R¹R^{1a}), cyclohexyl, phenyl, pyridinyl, norbonenyl, furanyl, pyrrolyl or thienyl,
wherein in case X¹ is a cyclic fragment, said cyclic fragment is incorporated into L of formula (X) via two adjacent ring atoms;
X² is a chemical bond or selected from C(R³R^{3a}), N(R³), O, C(R³R^{3a})—O or C(R³R^{3a})—C(R⁴R^{4a});
R¹, R³ and R⁴ are independently selected from H, C₁₋₄ alkyl and —N(R⁵R^{5a});
R^{1a}, R^{3a}, R^{4a} and R^{5a} are independently selected from H and C₁₋₄ alkyl;
R² is C₁₋₄ alkyl;
R⁵ is C(O)R⁶;
R⁶ is C₁₋₄ alkyl;

More preferably, L of formula (X) is selected from:

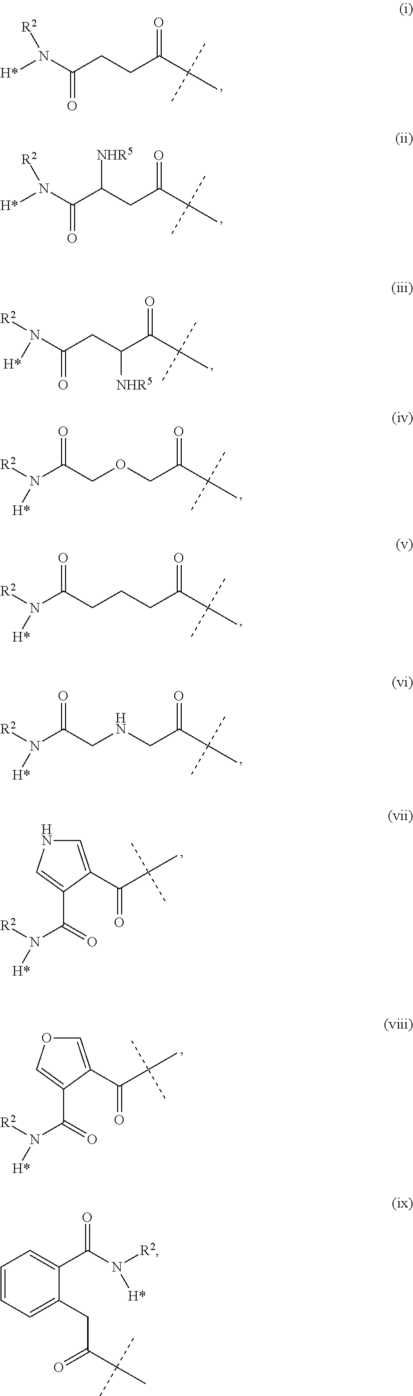

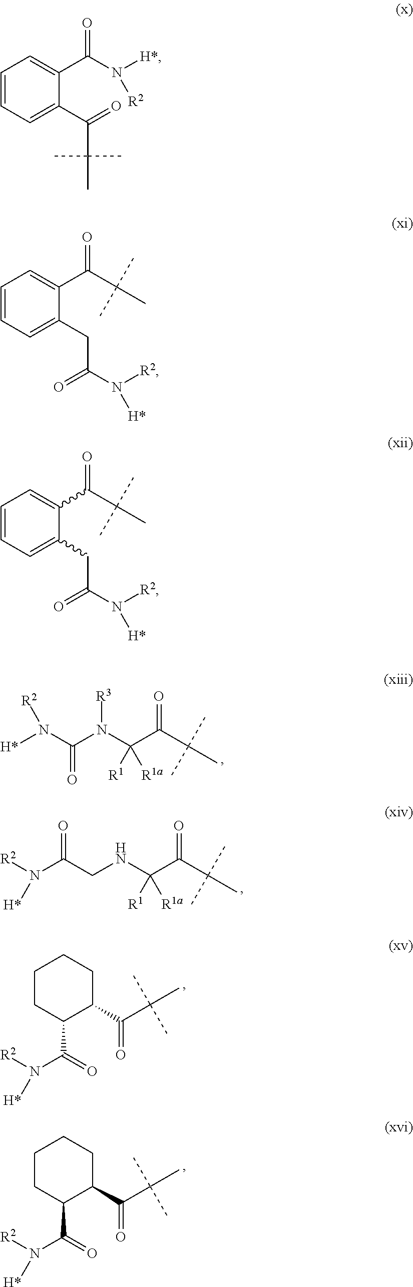

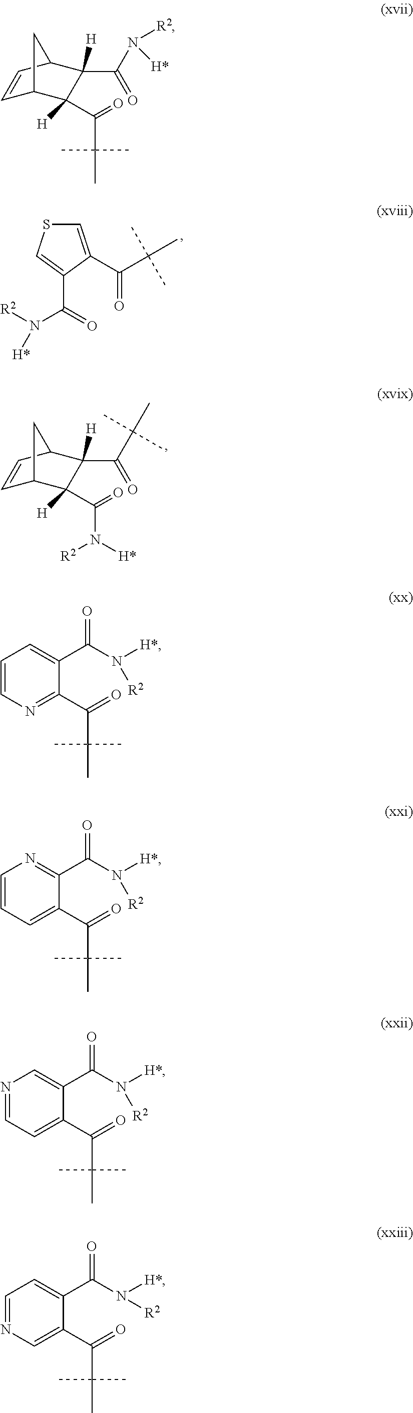

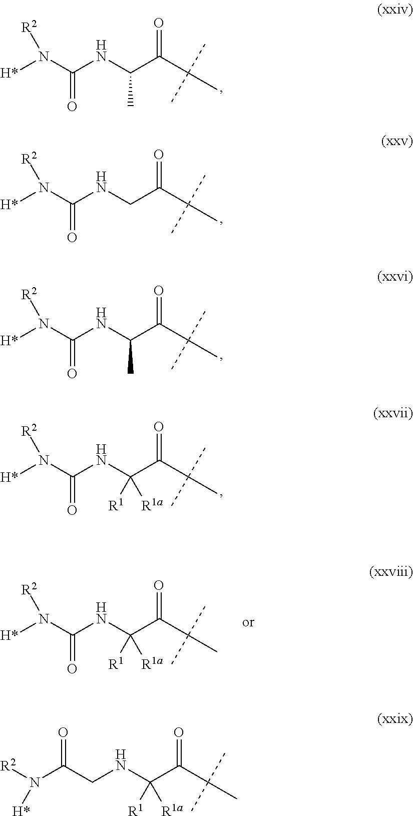

wherein the dashed line indicates attachment to D,
R⁵ is C(O)R⁶, and
R¹, R^{1a}, R², R³ and R⁶ are independently from each other C₁₋₄ alkyl.

L of formula (X) is substituted with one moiety [symbol] and preferably said substitution occurs at R², i.e. preferably R² is substituted with one moiety

[symbol]

Yet another preferred reversible prodrug linker moiety L for hydroxyl-comprising drugs is described in WO 2011/012721. Therefore, the following sub-structure of the general formula (XI) is a preferred embodiment for —(SP)ₓ-L-D for the water-soluble carrier-linked prodrug of the present invention according to formula (I):

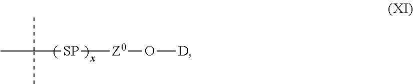

wherein the dashed line indicates attachment to a moiety Hyp of formula (I), which moiety Hyp of formula (I) is connected to m sub-structures of formula (XI),
D is connected through a hydroxyl group of D to the rest of the sub-structure of formula (XI), and
wherein D, SP, x and Z⁰ in formula (XI) have the following meaning:
D is a hydroxyl-comprising biologically active moiety D comprising O,
SP is the spacer moiety SP of formula (I),
x is 0 or 1,
Z⁰ is the moiety -L- of formula (I) and is X⁰—C(O), X⁰—O—C(O), X⁰—S(O)₂, X—C(S), X⁰—O—S(O)₂, X⁰—S(O)₂N(R¹), X⁰−CH(OR¹), X⁰—C(OR¹)(OR²), X⁰—C(O)N(R¹), X⁰—P(═O)OH)O, X⁰—P(═O)(OR¹)O, X⁰—P(═O)(SH)O, X⁰—P(═O)(SR¹)O, X⁰—P(═O)(OR¹), X⁰—P(═S)(OH)O, X⁰—P(═S)(OR¹)O, X⁰—P(═S)(OH)N(R¹), X⁰—P(═S)(OR¹)N(R²), X⁰—P(═O)(OH)N(R¹) or X⁰—P(═O)(OR¹)N(R²),
R¹, R² are independently selected from the group consisting of C₁₋₆ alkyl; or R¹ and R² jointly form a C₁₋₆ alkylene bridging group,
X⁰ is (X^{0A})ₘ₁—(X^{0B})ₘ₂,
m1, m2 are independently 0 or 1,
X^{0A} is T^{O},
X^{0B} is a branched or unbranched C₁₋₁₀ alkylene group which is unsubstituted or substituted with one or more R³, which is/are the same or different,
R³ is halogen, CN, C(O)R⁴, C(O)OR⁴, OR⁴, C(O)R⁴, C(O)N(R⁴R^{4a}), S(O)₂N(R⁴R^{4a}), S(O)N(R⁴R^{4a}), S(O)₂R⁴, S(O)R⁴, N(R⁴)S(O)₂N(R^{4a}R^{4b}), SR⁴, N(R⁴R^{4a}), NO₂, OC(O)R⁴, N(R⁴)C(O)R^{4a}, N(R⁴)SO₂R^{4a}, N(R⁴)S(O)R^{4a}, N(R⁴)C(O)N(R^{4a}R^{4b}), N(R⁴)C(O)OR^{4a}, OC(O)N(R⁴R^{4a}), or T⁰,
R⁴, R^{4a}, R^{4b} are independently selected from the group consisting of H, T⁰, C₁₋₄ alkyl, C₂₋₄ alkenyl, and C₂₋₄ alkynyl, wherein C₁₋₄ alkyl, C₂₋₄ alkenyl, and C₂₋₄ alkynyl are optionally substituted with one or more R⁵, which is/are the same of different,
R⁵ is halogen, CN, C(O)R⁶, C(O)OR⁶, OR⁶, C(O)R⁶, C(O)N(R⁶R^{6a}), S(O)₂N(R⁶R^{6a}), S(O)N(R⁶R^{6a}), S(O)₂R⁶, S(O)R⁶, N(R⁶)S(O)₂N(R^{6a}R^{6b}), SR⁶, N(R⁶R^{6a}), NO₂, OC(O)R⁶, N(R⁶)C(O)R^{6a}, N(R⁶)SO₂R^{6a}, N(R⁶)S(O)R^{6a}, N(R⁶)C(O)N(R^{6a}R^{6b}), N(R⁶)C(O)OR^{6a} or OC(O)N(R⁶R^{6a}),
R⁶, R^{6a}, R^{6b} are independently selected from the group consisting of H, C₁₋₆ alkyl, C₂₋₆ alkenyl, and C₂₋₆ alkynyl, wherein C₁₋₆ alkyl, C₂₋₆ alkenyl, and C₂₋₆ alkynyl are optionally substituted with one or more halogen, which is/are the same of different,
T^{O} is phenyl, naphthyl, azulenyl, indenyl, indanyl, C₃₋₇ cycloalkyl, 3- to 7-membered heterocyclyl, or 8- to 11-membered heterobicyclyl, wherein T⁰, is optionally substituted with one or more R⁷, which is/are the same or different,
R⁷ is halogen, CN, COOR⁸, OR⁸, C(O)R⁸, C(O)N(R⁸R^{8a}), S(O)₂N(R⁸R^{8a}), S(O)N(R⁸R^{8a}), S(O)₂R⁸, S(O)R⁸, N(R⁸)S(O)₂N(R^{8a}R^{8b}), SR⁸, N(R⁸R^{8a}), NO₂, OC(O)R⁸, N(R⁸)C(O)R^{8a}, N(R⁸)S(O)₂R^{8a}, N(R⁸)S(O)R^{8a}, N(R⁸)C(O)OR^{8a}, N(R⁸)C(O)N(R^{8a}R^{8b}), OC(O)N(R⁸R^{8a}), oxo (═O), where the ring is at least partially saturated, C₁₋₆ alkyl, C₂₋₆ alkenyl, or C₂₋₆ alkynyl, wherein C₁₋₆ alkyl, C₂₋₆ alkenyl, and C₂₋₆ alkynyl are optionally substituted with one or more R⁹, which is/are the same or different,
R⁸, R^{8a}, R^{8b} are independently selected from the group consisting of H, C₁₋₆ alkyl, C₂₋₆ alkenyl, and C₂₋₆ alkynyl, wherein C₁₋₆ alkyl, C₂₋₆ alkenyl, and C₂₋₆ alkynyl are optionally substituted with one or more R¹⁰, which is/are the same of different,
R⁹, R¹⁰ are independently selected from the group consisting of halogen, CN, C(O)R¹¹, C(O)OR¹¹, OR¹¹, C(O)R¹¹, C(O)N(R¹¹R^{11a}), S(O)₂N(R¹¹R^{11a}), S(O)N(R¹¹R^{11a}), S(O)₂R¹¹, S(O)R¹¹, N(R¹¹)S(O)₂N(R^{11a}R^{11b}), SR¹¹, N(R¹¹R^{11a}), NO₂, OC(O)R¹¹, N(R¹¹)C(O)R^{11a}, N(R¹¹)SO₂R^{11a}, N(R¹¹)S(O)R^{11a}, N(R¹¹)C(O)N(R^{11a}R^{11b}), N(R¹¹)C(O)OR^{11a}, and OC(O)N(R¹¹R^{11a}),
R¹¹, R^{11a}, R^{11b} are independently selected from the group consisting of H, C₁₋₆ alkyl, C₂₋₆ alkenyl, and C₂₋₆ alkynyl, wherein C₁₋₆ alkyl, C₂₋₆ alkenyl, and C₂₋₆ alkynyl are optionally substituted with one or more halogen, which is/are the same of different, and
wherein [symbol] of formula (XI) is covalently attached to X⁰.

Preferably, Z⁰ is X⁰—C(O), X⁰—C(O)O, or X⁰—S(O)₂. More preferably, Z⁰ is X⁰—C(O) or X⁰C(O)O. Even more preferably, Z⁰ is X⁰—C(O).

Preferably, X⁰ is unsubstituted.

Preferably, m1 is 0 and m2 is 1.

Preferably, X⁰ is C(R¹R²)CH₂, wherein R¹ and R² are independently selected from the group consisting of H and C₁₋₄ alkyl, provided that at least one of R¹, R² is other than H, or (CH₂)ₙ, wherein n is 3, 4, 5, 6, 7 or 8.

Preferably, the moiety [symbol] of formula (XI) is covalently attached to X⁰ via an amide group.

In yet another preferred embodiment the sub-structure —(SP)ₓ-L-D of formula (I) for the water-soluble carrier-linked prodrug of the present invention is of formula (XII):

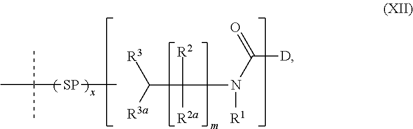

wherein the dashed line indicates attachment to a moiety Hyp of formula (I), which moiety Hyp of formula (I) is connected to m sub-structures of formula (XII),
D is connected through an aromatic hydroxyl group of D to the rest of the sub-structure of formula (XII) by forming a carbamate group,
the moiety [symbol] is attached to any one of R¹, R², R^{2a}, R³, and R^{3a}; and
wherein D, SP, x, R¹, R², R^{2a}, R³, R^{3a} and m in formula (XII) have the following meaning:
D is an aromatic hydroxyl-comprising biologically active moiety D,
SP is the spacer moiety SP of formula (I),
x is 0 or 1,
R¹ is selected from the group consisting of C₁₋₄ alkyl, heteroalkyl, C₃₋₇ cycloalkyl, and

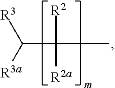

each R², each R^{2a}, R³, R^{3a} are independently selected from hydrogen, substituted or non-substituted linear, branched or cyclic C₁₋₄ alkyl or heteroalkyl,
m is 2, 3 or 4.

In the sub-structure —(SP)ₓ-L-D of formula (XII) the moiety L is of formula (XIIa):

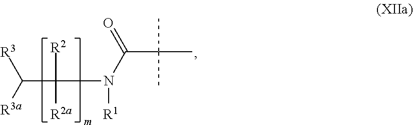

wherein
the dashed line indicates attachment to D of formula (XII), and
R¹, each R², each R^{2a}, R³, R^{3a} and m of formula (XIIa) are used as defined in formula (XII).

Optionally, L of formula (XII) is further substituted.

In yet another preferred embodiment the sub-structure —(SP)ₓ-L-D of formula (I) for the water-soluble carrier-linked prodrug of the present invention is given in formula (XIII):

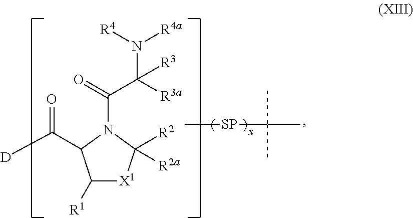

wherein the dashed line indicates attachment to a moiety Hyp of formula (I), which moiety Hyp of formula (I) is connected to m sub-structures of formula (XIII),
D is connected through an aliphatic amine group of D to the rest of the sub-structure of formula (XIII) by forming an amide group,
the moiety [symbol] is attached to any one of R¹, R², R^{2a}, R³, R^{3a}, R⁴, R^{4a}, and X¹; and
wherein D, SP, x, X₁, R¹, R², R^{2a}, R³, R^{3a}, R⁴ and R^{4a} in formula (XIII) have the following meaning:

D is an aromatic amine-comprising biologically active moiety D,
SP is the spacer moiety SP of formula (I),
x is 0 or 1,
X₁ is selected from O, S or CH—R^{1a}
R¹ and R^{1a} are independently selected from H, OH, CH₃,
R², R^{2a}, R⁴ and R^{4a} are independently selected from H and C₁₋₄ alkyl,
R³, R^{3a} are independently selected from H, C₁₋₄ alkyl, and R⁵,
R⁵ is selected from

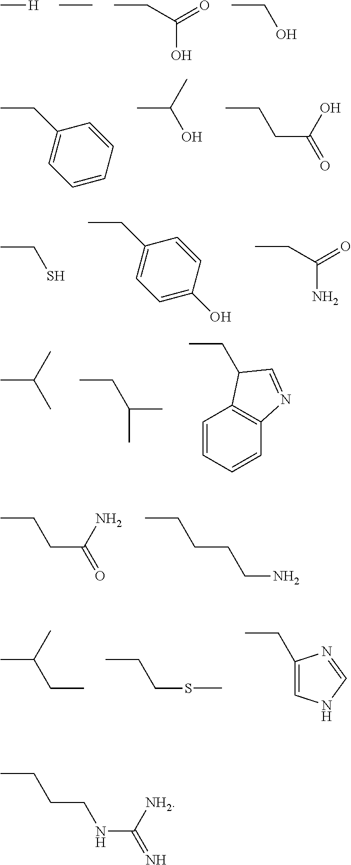

Preferably, one of the pair R³/R^{3a} of formula (XIII) is H and the other one is selected from R⁵.

Preferably, one of R⁴/R^{4a} of formula (XIII) is H.

Optionally, one or more of the pairs R³/R^{3a}, R⁴/R^{4a}, R³/R⁴ of formula (XIII) may independently form one or more cyclic fragment(s) selected from C₃₋₇ cycloalkyl, 4- to 7-membered heterocyclyl, and 9- to 11-membered heterobicyclyl.

Optionally, R³, R^{3a}, R⁴ and R^{4a} of formula (XIII) are further substituted. Suitable substituents are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4- to 7-membered heterocycle) or halogen moieties.

In the sub-structure —(SP)ₓ-L-D of formula (XIII) the moiety L is of formula (XIIIa):

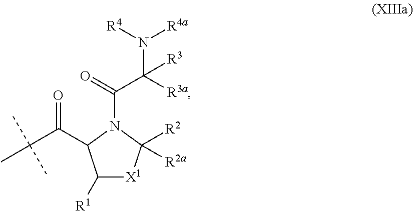

wherein
the dashed line indicates attachment to D of formula (XIII), and
X₁, R¹, R², R^{2a}, R³, R^{3a}, R⁴ and R^{4a} of formula (XIIIa) are used as defined in formula (XIII).

Optionally, L of formula (XIII) is further substituted. Suitable substituents are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4- to 7-membered heterocycle) or halogen moieties.

In yet another preferred embodiment the sub-structure —(SP)ₓ-L-D of formula (I) for the water-soluble carrier-linked prodrug of the present invention is of formula (XIV):

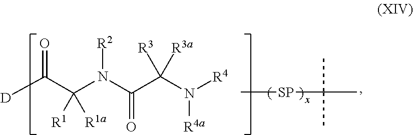

wherein the dashed line indicates attachment to a moiety Hyp of formula (I), which moiety Hyp of formula (I) is connected to m sub-structures of formula (XIV),
D is connected through an aromatic amine group of D to the rest of the sub-structure of formula (XIV) by forming an amide group,
the moiety [symbol] is attached to any one of R¹, R^{1a}, R², R³, R^{3a}, R⁴, and R^{4a}; and
wherein D, SP, x, R¹, R^{1a}, R², R^{2a}, R³, R^{3a}, R⁴ and R^{4a} in formula (XIV) have the following meaning:
D is an aromatic amine-comprising biologically active moiety D,
SP is the spacer moiety SP of formula (I),
x is 0 or 1:
R¹, R^{1a}, R², R³, R^{3a}, R⁴ and R^{4a} are independently selected from H and C₁₋₄ alkyl.

Optionally, any two of R¹, R^{1a}, R², R³, R^{3a}, R⁴ and R^{4a} of formula (XIV) may independently form one or more cyclic fragment(s) selected from C₃₋₇ cycloalkyl, 4- to 7-membered heterocyclyl, phenyl, naphthyl, indenyl, indanyl, tetralinyl, and 9- to 11-membered heterobicyclyl.

Optionally, R¹, R^{1a}, R², R³, R^{3a}, R⁴ and R^{4a} of formula (XIV) are further substituted. Suitable substituents are alkyl, such as C₁₋₆ alkyl, alkene, such as such as C₂₋₆ alkene, alkine, such as such as C₂₋₆ alkine, aryl, such as phenyl, heteroalkyl, heteroalkene, heteroalkine, heteroaryl such as aromatic 4- to 7-membered heterocycle, or halogen moieties.

In the sub-structure —(SP)ₓ-L-D of formula (XIV) the moiety L is of formula (XIVa):

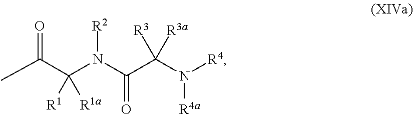

wherein
the dashed line indicates attachment to D of formula (XIV), and
R¹, R^{1a}, R², R^{2a}, R³, R^{3a}, R⁴ and R^{4a} of formula (XIVa) are used as defined in formula (XIV).

Optionally, L of formula (XIV) is further substituted. Suitable substituents are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4- to 7-membered heterocycle) or halogen moieties.

Preferably, one of R⁴ or R^{4a} of formula (XIV) is H.

Yet another preferred reversible prodrug linker moiety L is described in U.S. Pat. No. 7,585,837. Therefore, the following sub-structure of the general formula (XV) is a preferred embodiment for —(SP)ₓ-L-D for the water-soluble carrier-linked prodrug of the present invention according to formula (I):

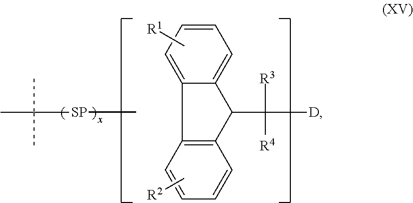

wherein the dashed line indicates attachment to a moiety Hyp of formula (I), which moiety Hyp of formula (I) is connected to m sub-structures of formula (XV),
D is connected through a functional group of D to the rest of the sub-structure of formula (XV), wherein such functional group is selected from amine, carboxyl, phosphate, hydroxyl and mercapto,
the moiety [symbol] is attached to any one of R¹, R², R³, and R⁴; and
wherein D, SP, x, R¹, R², R³ and R⁴ in formula (XV) have the following meaning:
D is an aromatic amine-comprising biologically active moiety D,
SP is the spacer moiety SP of formula (I),
x is 0 or 1,
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂,
R³, R⁴, and R⁵ are independently selected from the group consisting of hydrogen, alkyl, and aryl.

In the sub-structure —(SP)ₓ-L-D of formula (XV) the moiety L is of formula (XVa):

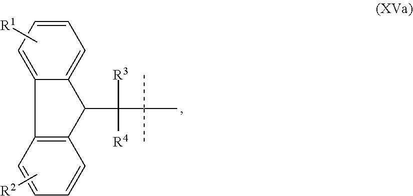

wherein
the dashed line indicates attachment to D of formula (XV), and
R¹, R², R³ and R⁴ of formula (XVa) are used as defined in formula (XV).

Optionally, L of formula (XV) is further substituted. Suitable substituents are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Yet another preferred reversible prodrug linker moiety L is described in the international application WO-A 2002/089789. Therefore, the following sub-structure of the general formula (XVI) is a preferred embodiment for —(SP)ₓ-L-D for the water-soluble carrier-linked prodrug of the present invention according to formula (I):

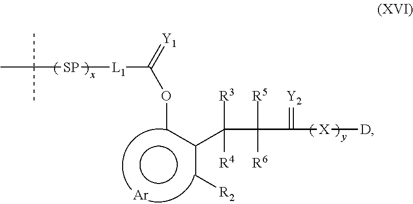

wherein the dashed line indicates attachment to a moiety Hyp of formula (I), which moiety Hyp of formula (I) is connected to m sub-structures of formula (XVI),
D is connected through a functional group of D to the rest of the sub-structure of formula (XVI),
and wherein SP, x, D, X, Ar, L₁, Y₁, Y₂, y, R², R³, R⁴, R⁵, and R⁶ of formula (XVI) have the following meaning:
D is a biologically active moiety,
SP is the spacer moiety SP of formula (I),
x is 0 or 1,
y is 0or 1,
L₁ is a bifunctional linking group,
Y₁ and Y₂ are independently O, S or NR⁷,
R¹⁻⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy,
Ar is a moiety which when included in formula (XVI) forms a multisubstituted aromatic hydrocarbon or a multi-substituted heterocyclic group,
X is a chemical bond or a moiety that is actively transported into a target cell, a hydrophobic moiety, or a combination thereof.

Yet another preferred reversible prodrug linker moiety L is described in the international application WO-A 2001/47562. Therefore, the following sub-structure of the general formula (XVII) is a preferred embodiment for —(SP)ₓ-L-D for the water-soluble carrier-linked prodrug of the present invention according to formula (I):

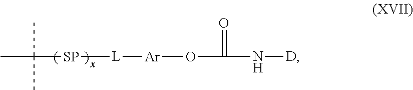

wherein the dashed line indicates attachment to a moiety Hyp of formula (I), which moiety Hyp of formula (I) is connected to m sub-structures of formula (XVII),
D is connected through an amine group of D to the rest of the sub-structure of formula (XVII),
and wherein SP, x, D, L and Ar of formula (XVII) have the following meaning:
D is an amine-comprising biologically active moiety comprising NH,
SP is the spacer moiety SP of formula (I),
x is 0 or 1,
L is a covalent linkage, preferably a hydrolytically stable linkage,
Ar is an aromatic group.

Yet another preferred reversible prodrug linker moiety L is described in U.S. Pat. No. 7,393,953 B2. Therefore, the following sub-structure of the general formula (XVIII) is a preferred embodiment for —(SP)ₓ-L-D for the water-soluble carrier-linked prodrug of the present invention according to formula (I):

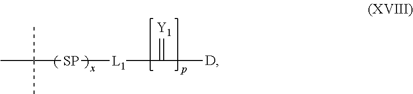

wherein the dashed line indicates attachment to a moiety Hyp of formula (I), which moiety Hyp of formula (I) is connected to m sub-structures of formula (XVIII),
D is connected through a heteroaromatic amine group of D to the rest of the sub-structure of formula (XVIII),
and wherein SP, x, D, L₁, Y₁ and p of formula (XVIII) have the following meaning:
D is a heteroaromatic amine-comprising biologically active moiety,
SP is the spacer moiety SP of formula (I),
x is 0 or 1,
Y₁ is O, S, or NR₂,
p is 0 or 1,
L₁ is a bifunctional linker, such as, for example, —NH(CH₂CH₂O)ₘ(CH₂)ₘNR₃—, —NH(CH₂CH₂O)ₘC(O)—, —NH(CR₄R₅)ₘOC(O)—, —C(O)(CR₄R₅)ₘNHC(O)(CR₈R₇)_{q}NR₃, —C(O)O(CH₂)ₘO—, —C(O)(CR₄R₅)ₘNR₃—, —C(O)NH(CH₂CH₂O)ₘ(CH₂)ₘNR₃—, —C(O)O—(CH₂CH₂O)ₘNR₃—, —C(O)NH(CR₄R₅)ₘO—, —C(O)O(CR₄R₅)ₘO, —C(O)NH(CH₂CH₂O)ₘ—,

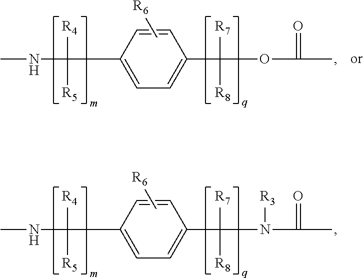

R₂, R₃, R₄, R₅, R₇ and R₈ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy,
R₆ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, NO₂, haloalkyl and halogen,
m and q are selected independently from each other and each is a positive integer.

In yet another preferred embodiment the sub-structure —(SP)ₓ-L-D of formula (I) for the water-soluble carrier-linked prodrug of the present invention is given in formula (XIX):

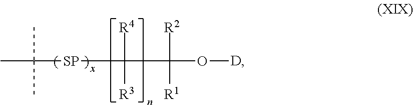

wherein the dashed line indicates attachment to a moiety Hyp of formula (I), which moiety Hyp of formula (I) is connected to m sub-structures of formula (XIX),
D is connected through a carboxyl group of D to the rest of the sub-structure —(SP)ₓ-L- of formula (I) by forming a carboxylic ester comprising O,
and wherein SP, x, D, R¹, R², R³, R⁴ and n of formula (XIX) have the following meaning:
D is a carboxyl-comprising biologically active moiety,
SP is the spacer moiety SP of formula (I),
x is 0 or 1,
R¹ is selected from the group of unsubstituted alkyl; substituted alkyl; unsubstituted phenyl; substituted phenyl; unsubstituted naphthyl; substituted naphthyl; unsubstituted indenyl; substituted indenyl; unsubstituted indanyl; substituted indanyl; unsubstituted tetralinyl; substituted tetralinyl; unsubstituted C₃₋₁₀ cycloalkyl; substituted C₃₋₁₀ cycloalkyl; unsubstituted 4- to 7-membered heterocyclyl; substituted 4- to 7-membered heterocyclyl; unsubstituted 9- to 11-membered heterobicyclyl; and substituted 9- to 11-membered heterobicyclyl;
R² is selected from H, unsubstituted alkyl, and substituted alkyl;
R³ and R⁴ are independently selected from the group consisting of H, unsubstituted alkyl, and substituted alkyl;
n is 0 or 1,
optionally, R¹ and R³ are joined together with the atoms to which they are attached to form a ring A,
A is selected from the group consisting of C₃₋₁₀ cycloalkyl; 4- to 7-membered aliphatic heterocyclyl; and 9- to 11-membered aliphatic heterobicyclyl, wherein A is unsubstituted or substituted.

Preferably, R¹ of formula (XIX) is C₁₋₆ alkyl or substituted C₁₋₆ alkyl, more preferably C₁₋₄ alkyl or substituted C₁₋₄ alkyl.

More preferably, R¹ of formula (XIX) is selected from methyl, ethyl, n-propyl, isopropyl, n-butyl, isobutyl, sec-butyl, t-butyl, and benzyl.

Preferably, R² of formula (XIX) is H.

Preferably, R³ of formula (XIX) is H, C₁₋₆ alkyl or substituted C₁₋₆ alkyl, more preferably C₁₋₄ alkyl or substituted C₁₋₄ alkyl. More preferably, R³ is selected from methyl, ethyl, n-propyl, isopropyl, n-butyl, isobutyl, sec-butyl, t-butyl, and benzyl.

More preferably, R³ of formula (XIX) is H.

Preferably, R⁴ of formula (XIX) is s H, C₁₋₆ alkyl or substituted C₁₋₆ alkyl, more preferably C₁₋₄ alkyl or substituted C₁₋₄ alkyl. More preferably, R⁴ is selected from methyl, ethyl, n-propyl, isopropyl, n-butyl, isobutyl, sec-butyl, t-butyl, and benzyl.

More preferably, R⁴ of formula (XIX) is H.

In another preferred embodiment, R¹ and R³ of formula (XIX) are joined together with the atoms to which they are attached to form a ring A, wherein A is selected from the group consisting of cyclopropane, cyclobutane, cyclopentane, cyclohexane, and cycloheptane.

In yet another preferred embodiment the sub-structure —(SP)ₓ-L-D of formula (I) for the water-soluble carrier-linked prodrug of the present invention is given in formula (XX):

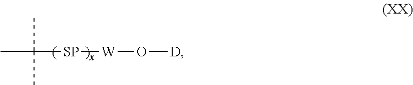

wherein the dashed line indicates attachment to a moiety Hyp of formula (I), which moiety Hyp of formula (I) is connected to m sub-structures of formula (XX),
D is connected through a carboxyl group of D to the rest of the sub-structure of formula (XX) by forming a carboxylic ester comprising O,
and wherein SP, x, D, and W of formula (XX) have the following meaning:
D is a carboxyl-comprising biologically active moiety,
SP represents the spacer moiety SP of formula (I),
x is 0 or 1:
W is selected from linear C₁₋₁₅ alkyl.

Preferably, a carrier moiety of the water-soluble carrier-linked prodrug of formula (I) is connected to at least 6 moieties L (either directly or indirectly), such as to 6, 7, 8, 9, 10, 11, 12, 13, 14, 15, 16, or 17 moieties L (either directly or indirectly). More preferably, a carrier moiety of the water-soluble carrier-linked prodrug of formula (I) is connected to 8, 12, 16 or 20 moieties L (either directly or indirectly).

Preferably, all moieties L of formula (I) are the same.

A water-soluble carrier-linked prodrug of formula (I) comprises biologically active moieties D which are preferably selected from the group of oligopeptides, polypeptides, proteins, oligonucleotides, and small molecule biologically active moieties. The corresponding drugs may comprise one or more functional groups selected from the group comprising amine, hydroxyl, carboxyl, phosphate, and mercapto. A drug may be conjugated to a moiety L through a linkage formed by an amine, such as an aliphatic or aromatic amine, hydroxyl, such as an aliphatic or aromatic hydroxyl, carboxyl, phosphate, or mercapto group provided by the drug.

Suitable aromatic amine-containing drugs are, for example, (−)-Carbovir, (±)-Hymenin, (±)-Norcisapride, (±)-Picumeterol, (R)-Aminoglutethimide, (R)-Clenbuterol, (S)-Aminoglutethimide, (S)-Clenbuterol, [6-p-aminophenylalanine]-angiotensin II, 10′-Demethoxystreptonigrin, 17-Aminogeldanamycin, 1-Aminoacridine, 1-Deazaadenine, 1-NA-PP 1, 1-NM-PP 1, 2,7-Diaminoacridine, 2,7-Dimethylproflavine, 2-Amino-6(5H)-phenanthridinone, 2-Aminoacridine, 2-amino-Carbanilide, 2-Aminohistamine, 2-Aminoperimidine, 2′-AMP, 2-Chloroadenosine, 2′-Deoxyxylotubercidin, 2-Sulfanilamidoimidazole, 3,4-Diaminocoumarin, 3′-Amino-4′-methoxyflavone, 3-Aminoacridine, 3-Aminopicolinic acid, 3-Deazaguanine, 4′-Aminoflavone, 4-Aminopyridine, 5′-ADP, 5-Aminoacridine, 5-amino-DL-Tryptophan, 5-Aminonicotinamide, 5′-AMP, 5′-ATP, 5-Chlorodeoxycytidine, 5′-CMP, 5-Dimethylamiloride, 5′-GDP, 5′-GMP, 5′-GTP, 5-Iodotubercidin, 5-Methylcytosine, 6-Aminoflavone, 6-Aminophenanthridine, 6-Aminothymine, 6-Benzylthioguanine, 6-Chlorotacrine, 6-Iodoamiloride, 7,8-Dihydroneopterin, 7-Aminonimetazepam, 7-Methoxytacrine, 7-Methyltacrine, 9-Deazaguanine, 9-Phenethyladenine, Abacavir, Acadesine, Acediasulfone, Acefurtiamine, Acetyl coenzyme A, Aciclovir, Actimid, Actinomycin, Acyclovir, Adefovir, Adenallene, Adenine, Adenophostin A, Adenosine, Adenosine monophosphate, Adenosine triphosphate, Adenosylhomocysteine, Aditeren, Afloqualone, Alamifovir, Albofungin, Alfuzosin, Allithiamine, Alpiropride, Amanozine, Ambasilide, Ambucaine, Amdoxovir, Ameltolide, Amethopterin, Amfenac, Amflutizole, Amicycline, Amidapsone, Amifampridine, Amiloride, Aminacrine, Aminoacridine, Aminoantipyrine, Aminobenzoate, Aminogenistein, Aminoglutethimide, Aminohippurate, Aminoisatin, Aminometradine, Aminonimetazepam, Aminophenylalanine, Aminopotentidine, Aminopterin, Aminopurvalanol A, Aminoquinuride, Aminosalicylic Acid, Amiphenazole, Amiphenosine, Amisometradine, Amisulpride, Amiterol, Amlexanox, Ammelin, Amonafide, Amoxecaine, Amphenidone, Amphethinile, Amphotalide, Amprenavir, Ampurine, Amrinone, AMT, Amthamine, Amtizole, Angustmycin A, Anileridine, Apadenoson, Apraclonidine, Apricitabine, Arafluorocytosine, Aramine, Arazide, Aristeromycin, Arprinocid, Ascamycin, Ascensil, Aspiculamycin, Atolide, Azabon, Azacitidine, Azaline B, Azamulin, Azanidazole, Azepexole, Aztreonam, Baquiloprim, Basedol, Batanopride, b-D-Adenosine, Bemitradine, Benfotiamine, Bentiamine, Benzamil, Benzocaine, Betoxycaine, Binodenoson, Biopterin, Bisbentiamine, Blasticidin, Bleomycin, Bleomycin A1, Bleomycin A2, Bleomycin A5, Bleomycin A6, Bleomycin DMA2, Brodimoprim, Bromfenac, Bromobuterol, Bromopride, Bropirimine, Buciclovir, Bunazosin, Butyrylthiamine disulfide, Cadeguomycin, cAMP, Candicidin, Capadenoson, Carbanilide, Carbodine, Carbovir, Carbutamide, Carumonam, CDP-dipalmitin, Cefcapenepivoxil, Cefclidin, Cefdaloxime, Cefdinir, Cefditoren, Cefempidone, Cefepime, Cefetamet, Cefetecol, Cefixime, Cefluprenam, Cefmatilen, Cefmenoxime, Cefodizime, Cefoselis, Cefotaxime, Cefotiam, Cefozopran, Cefpodoxime, Cefquinome, Cefrom, Ceftazidime, Cefteram, Ceftibuten, Ceftiofur, Ceftiolene, Ceftioxide, Ceftizoxime, Ceftobiprole, Ceftriaxone, Cefuzonam, Centazolone, Cetotiamine, cGMP, Chloroprocaine, Cidofovir, Cifostodine, Cipamfylline, Cisapride, Cladribine, Clafanone, Claforan, Clebopride, Clenbuterol, Clenproperol, Clofarabine, Clorsulon, Coelenteramine, Coenzyme A, Colchicamid, Coumarin 10, Coviracil, Crotonoside, Cyclobut A, Cyclobut G, Cycloclenbuterol, Cycotiamine, Cytallene, Cytarabine, Cytarazid, Cytidine, Cytidine diphosphate, Cytidoline, CytosineD-(+)-Neopterin, Dactinomycin, D-Amethopterin, dAMP, Damvar, Daniquidone, Dapsone, Daptomycin, Daraprim, Darunavir, DATHF, Dazopride, dCMP, dCTP, Debromohymenialdisine, Decitabine, Declopramide, Deisopropylhydroxyatrazine, Delafloxacin, Delfantrine, Denavir, Deoxyadenosine, Deoxy-ATP, Deoxycytidine, Deoxyguanosine, Dephosphocoenzyme A, Dequalinium, Desbutylbumetanide, Desciclovir, Desoxyminoxidil, dGMP, dGTP, Diacethiamine, Diaminoacridine, Diaveridine, Dichlorobenzamil, Dichloromethotrexate, Dichlorophenarsine, Dideoxycytidine, Dihydrobiopterin, Dihydrofolic acid, Dimethialium, Dimethocaine, Dimethyl methotrexate, Dinalin, DL-5,6,7,8-Tetrahydrofolic acid, DL-Methotrexate, Dobupride, Dovitinib, Doxazosin, Draflazine, Edatrexate, Elpetrigine, Elvucitabine, Emtricitabine, Entecavir, Enviradene, Epcitabine, Epiroprim, Eritadenine, Etanterol, Ethacridine, Ethaden, Ethylisopropylamiloride, Etoprine, Etoxazene, Etravirine, Etriciguat, FAD, Famciclovir, Fazarabine, Fenamol, Fepratset, Fiacitabine, Flucytosine, Fludara, Fludarabine, Fluocytosine, Folic acid, Formycin A, Fosamprenavir, Furalazine, Fursultiamine, Furyltriazine, Ganciclovir, Gancyclovir, Gastracid, Gemcitabine, Giracodazole, Gloximonam, Glybuthiazol, GSK 3B Inhibitor XII, GSK3BInhibitorXII, Guanine, Guanine arabinoside, Guanosine, Hexyl PABA, Hydroxymethylclenbuterol, Hydroxyprocaine, Hydroxytriamterene sulfate, Ibacitabine, Iclaprim, Imanixil, Imiquimod, Indanocine, Iobenzamic acid, Iocetamic acid, Iomeglamic acid, Iomeglamicacid, Ipidacrine, Iramine, Irsogladine, Isatoribine, Isobutamben, Isoritmon, Isosepiapterin, Ketoclenbuterol, Ketotrexate, Kopexil, Lamivudine, Lamotrigin, Lamotrigine, Lamtidine, Lappaconine, Lavendamycin, L-Cytidine, Lenalidomide, Leucinocaine, Leucovorin, L-g-Methylene-10-deazaaminopterin, Linifanib, Lintopride, Lisadimate, Lobucavir, Lodenosine, Lomeguatrib, Lometrexol, Loxoribine, L-S-Adenosylmethionine, Mabuterol, Medeyol, Melarsenoxyd, Melarsoprol B, Mesalazine, Metabutethamine, Metabutoxycaine, Metahexamide, Metazosin, Methioprim, Methotrexate, Methylanthranilate, Metioprim, Metoclopramide, Metoprine, Minoxidil, Mirabegron, Mitomycin, Mivobulin, Mocetinostat, Monocain, Mosapride, Mutamycin, N-(p-Aminophenethyl)spiroperidol, N6-[2-(4-aminophenyl)ethyl]adenosine Role, NAD+, NADH, NADH2, NADP+, NADPH2, Naepaine, Naminterol, Naretin, Nebidrazine, NECA, Nelarabine, Nelzarabine, Neolamin, Neotropine, Nepafenac, Nerisopam, Neurofort, Nifurprazine, Nimustine, Nitrine, N-Methyltetrahydrofolic acid, Nolatrexed, Nomifensine, Norcisapride, N-Propionylprocainamide, N-Sulfanilylnorfloxacin, o-Aminophenylalanine, Octotiamine, Olamufloxacin, Ormetoprim, Orthocaine, Oximonam, Oxybuprocaine, p-Aminoantipyrine, p-Aminobenzoate, p-Amino-D-phenylalanine, Pancopride, Parsalmide, Pasdrazide, Pathocidine, Pelitrexol, Pemetrexed, Penciclovir, Peplomycin, Peralopride, Phenamil, Phenazone, Phenazopyridine, Phenyl p-aminobenzoate, Phenyl-PAS-Tebamin, Phleomycin D1, Pibutidine, Picumeterol, Pirazmonam, Piridocaine, Piritrexim, Porfiromycin, Pralatrexate, Pramipexole, Prazobind, Prazosin, Preladenant, Procainamide, Procaine, Proflavine, Proparacaine, Propoxycaine, Prosultiamine, Prucalopride, Pseudoisocytidine, Psicofuranine, Pteridoxamine, Pteroyltriglutamic acid, Pyramine, Pyrimethamine, Questiomycin, Quinelorane, Racivir, Regadenoson, Renoquid, Renzapride, Resiquimod, Resorcein, Retigabine, Reverset, Riluzole, Rociclovir, Rufocromomycin, S-Adenosylmethionine, Sangivamycin, Sapropterin, S-Doxazosin, Sepiapterine, Silversulfadiazine, Sinefungin, Sipatrigine, Sparfloxacin, Sparsomycin, Stearyl-CoA, Stearylsulfamide, Streptonigrin, Succisulfone, Sufamonomethoxine, Sulamserod, Sulfabromomethazine, Sulfacetamide, Sulfachlorpyridazine, Sulfachrysoidine, Sulfaclomide, Sulfaclorazole, Sulfaclozine, Sulfacytine, Sulfadiasulfone, Sulfadiazine, Sulfadicramide, Sulfadimethoxine, Sulfadimidine, Sulfadoxine, Sulfaethoxypyridazine, Sulfaguanidine, Sulfaguanole, Sulfalene, Sulfamerazine, Sulfamethazine, Sulfamethizole, Sulfamethoxazole, Sulfamethoxydiazine, Sulfamethoxypyridazine, Sulfametomidine, Sulfametopyrazine, Sulfametrole, Sulfanilamide, Sulfanilamidoimidazole, Sulfanilylglycine, Sulfaperin, Sulfaphenazole, Sulfaproxyline, Sulfapyrazole, Sulfapyridine, Sulfasomizole, Sulfasymazine, Sulfathiadiazole, Sulfatroxazole, Sulfatrozole, Sulfisomidine, Sulfisoxazole, Tacedinaline, Tacrine, Talampanel, Talipexole, Talisomycin A, Tenofovir, Tenofovir disoproxil, Terazosin, Tetrahydrobiopterinm, Tetrahydrofolic acid, Tetroxoprim, Tezacitabine, Thiamine, Thiazosulfone, Thioguanine, Tiamiprine, Tigemonam, Timirdine, Tinoridine, Tiodazosin, Tirapazamine, Tiviciclovir, Tocladesine, Trancopal, Triacanthine, Triamterene, Triapine, Triciribine, Trimazosin, Trimethoprim, Trimetrexate, Tritoqualine, Troxacitabine, Tubercidin 5′-diphosphate, Tuvatidine, Tyrphostin AG 1112, Valacyclovir, Valganciclovir, Valopicitabine, Valtorcitabine, Velnacrine, Vengicide, Veradoline, Vidarabine, Viroxime, Vitaberin, Zalcitabine, Zhengguangmycin B2, Zinviroxime, Zorbamycin, Zoxazolamine, (±)-Saxitoxin, 2-Aminoperimidine, 6-Formylpterin, 8-13-Neurotensin, 8-Thioguanosine, 9-Deazaguanosine, 9-Desarginine-bradykinin, a4-10-Corticotropin, Afamelanotide, Agmatine, Alarelin, Ambazone, Amiloride, Aminopterine, Ampyrimine, Angiotensin, Angiotensin I, Angiotensin II, Antibiotic O-129, Antipain, Arginine, Argiprestocin, Astressin, Atriopeptin III, Aviptadil, Benzylisothiourea, Betacyamine, Bisindolylmaleimide IX, Bivalirudin, Blasticidin S, Bleomycin B2, Bombesin 14, Buformin, Camostat, Cariporide, Carperitide, Cecropin P 1, Cetrorelix, Cilengitide, Creapure, Cyanoginosin LR, Cyanoviridin RR, Dalargine, Damvar, Deazaaminopterin, Defensin HNP 1, Deslorelin, Desmopressin, Dezaguanine, Dichloromethotrexate, Dihydrostreptomycin, Dimaprit, Dimethylamiloride, Diminazene, DL-Methotrexate, D-Methotrexate, Ebrotidine, Edatrexate, Eel Thyrocalcitonin, Elastatinal, Elcatonin, Enterostatin, Enviomycin, Eptifibatide, Ethylisopropylamiloride, Etilamide, Etoprine, Famotidine, Flupirtine, Furterene, Galanin, Galegin, Ghrelin, Glucagon, Gonadoliberin A, Guanethidine, Guanfacine, Guanoxan, Guanylthiourea, Gusperimus, Hexamidine, Histatin 5, Histrelin, Homoarginine, Icatibant, Imetit, Insulinotropin, Isocaramidine, Kallidin 10, Kemptide, Ketotrexate, Kiotorphin, Lactoferricin, Lamifiban, L-Bradykinin, Leucoverin, Leucovorin A, Leupeptin, Leuprolide, Lometrexol, Lutrelin, m-Chlorophenylbiguanide, Melagatran, Melanotan II, Melanotropin, Melittin, Metformin, Methotrexate dimethyl ester, Methotrexate monohydrate, Methoxtrexate, Methylisothiourea, Metoprine, Miacalcin, MIBG, Minoxidil, Mitoguazone, Mivobulin, Mivobulin isethionate, Moroxydine, Nafarelin, Neotine, Nesiritide, Netropsin, Neurotensin, N-Methyltetrahydrofolate, Nociceptin, Nolatrexed, Novastan, Panamidin, Pathocidine, Pebac, Peldesine, Pelitrexol, Pemetrexed, Pentamidine, Peramivir, Phenformine, Phenylbiguanide, Pig galanin, Pimagedine, Piritrexim, Pitressin, Porcine angiotensinogen, Porcine gastrin-releasing hormone, Porcine neuropeptide Y, Porcine PHI, Pralatrexate, Protein Humanin, Proteinase inhibitor E 64, Pyrimethamin, Quinespar, Rat atriopeptin, Rat atriopeptin, Resiquimod, Ribamidine, Rimorphin, Saralasin, Saxitoxin, Sermorelin, S-Ethylisothiourea, Spantide, Stallimycin, Stilbamidine, Streptomycin A, Substance P free acid, Sulfaguanidine, Synthetic LH-releasing hormone, Tallimustine, Teprotide, Tetracosactide, Tetrahydrobiopterin, Tetrahydrofolic acid, Thrombin receptor-activating peptide-14, Thymopentin, Tioguanin, Tiotidine, Tirapazamine, Triamteren, Trimetrexate, Tryptorelin, Tuberactinomycin B, Tuftsin, Urepearl, Viomycidin, Viprovex, Vitamin M, Xenopsin, Zanamivir, Zeocin, Ziconotide, Zoladex.

Suitable drugs with an amine group may be selected from the group consisting of Aphidicolin Glycinate, Cetrorelix Acetate, Picumeterol Fumarate, (−)-Draflazine, (−)-Indocarbazostatin B, (+)-(23,24)-Dihydrodiscodermolide, (+)-(R)-Pramipexole, (R)-(+)-Amlodipine, (R)-(+)-Terazosin, (R)-Ganciclovir Cyclic Phosphonate, (R)-Sufinosine, (R)-Zacopride, (S)-(−)-Norketamine, (S)-Oxiracetam, (S)-Sufinosine, (S)-Zacopride Hydrochloride, [90Y]-DOTAGA-Substance P, [ARG(Me)9] MS-10, [D-TYR1,ARG(Me)9] MS-10, [D-TYR1,AzaGLY7,ARG(Me)9] MS-10, [D-TYR1] MS-10, [Psi(CH2NH)TPG4]Vancomycin Aglycon, [TRP19] MS-10, 111IN-Pentetreotide, 13-Deoxyadriamycin Hydrochloride, 17-Aminogeldanamycin, 19-O-Methylgeldanamycin, 1-Methyl-D-Tryptophan, 21-Aminoepothilone B, 2-Aminoaristeromycin, 2-Aminoneplanocin A, 3-Chloroprocainamide, 3-Deazaadenosine, 3-Matida, 4-Aminosalicylic Acid, 4-Chlorophenylthio-DADME-Immucillin-A, 5,4′-Diepiarbekacin, 5′-Homoneplanocin A, 5-Aminosalicylic Acid, 8(R)-Fluoroidarubicin Hydrochloride, 99MTC-C(RGDFK*)2Hynic, 9-Aminocamptothecin, A-42867 Pseudoaglycone, Abacavir Succinate, Abacavir Sulfate, Abanoquil Mesilate, Abarelix, Acadesine, Acriflavine, Acyclovir, Acyclovir Elaidate, Acyclovir Oleate, Acyline, Adefovir, Adefovir Dipivoxil, Ademetionine Tosylate Sulfate, Adenallene, Adenophostin A, Adenophostin B, Adenosine, Aerothricin 1, Aerothricin 16, Aerothricin 41, Aerothricin 45, Aerothricin 5, Aerothricin 50, Aerothricin 55, Afloqualone, Ageliferin Diacetate, Ageliferin Dihydrochloride, Aladapcin, Alamifovir, Alatrofloxacin Mesilate, Alendronic Acid Sodium Salt, Alestramustine, Alfuzosin Hydrochloride, Aliskiren Fumarate, Alogliptin Benzoate, Alpha-Methylnorepinephrine, Alpha-Methyltryptophan, Altemecidin, Alvespimycin Hydrochloride, Amantadine Hydrochloride, Ambasilide, Ambazone, Ambroxol Nitrate, Amdoxovir, Ameltolide, Amelubant, Amezinium Methylsulfate, Amfenac Sodium, Amidox, Amifostine Hydrate, Amikacin, Amiloride Hydrochloride, Aminocandin, Aminoglutethimide, Aminoguanidine, Aminolevulinic Acid Hexyl Ester, Aminolevulinic Acid Methyl Ester, Amisulpride, Amlodipine, Amlodipine Besylate, Amoxanox, Amoxicillin Pulsys, Amphotericin B, Ampicillin Sodium, Amprenavir, Ampydin, Amrinone, Amrubicin Hydrochloride, Amselamine Hydrobromide, Amthamine, Anakinra, Anamorelin Hydrochloride, Anatibant Mesilate, Angiopeptin Acetate, Anisperimus, Antagonist-G, Antide, Antide-1, Antide-2, Antide-3, Antileukinate, Apadenoson, Apixaban, Aplonidine Hydrochloride, Apoptozole 1, Apoptozole 2, Apoptozole 3, Apricitabine, Arbekacin, Arbekacin sulfate, Arborcandin A, Arborcandin B, Arborcandin C, Arborcandin D, Arborcandin E, Arborcandin F, Argatroban Monohydrate, Argimesna, Arginine Butyrate, Argiotoxin-636, Armodafinil, Arotinolol Hydrochloride, Arterolane Maleate, Aspoxicillin, Atenolol, Atosiban, Atreleuton, Avorelin, Azacytidine, Azalanstat, Azaromycin SC, Azelnidipine, Azetirelin, Azodicarbonamide, Azoxybacilin, Aztreonam, Aztreonam L-Lysine, Azumamide A, Baclofen, Bactobolin, Balapiravir Hydrochloride, Balhimycin, Barusiban, Batracylin, Belactin A, Belactosin A, Belactosin C, Benanomicin B, Benexate Cyclodextrin, Benzocaine, Besifloxacin Hydrochloride, Beta-Amyloid (12-20), Binodenoson, Bleomycin A2 Sulfate, Boceprevir, Bogorol A, Boholmycin, Brasilicardin A, Bremelanotide, Brivanib Alaninate, Brivaracetam, Brodimoprim, Bromfenac Sodium, Bromhexine Hydrochloride, Brostallicin Hydrochloride, Bunazosin Hydrochloride, Buserelin Acetate, Butabindide, Butamidine, Buteranol, Cabin 1, Calcium-Like Peptide 1, Calcium-Like Peptide 2, Cambrescidin 800, Cambrescidin 816, Cambrescidin 830, Cambrescidin 844, Camostat, Canfosamide Hydrochloride, Capadenoson, Capeserod Hydrochloride, Capravirine, Caprazamycin A, Caprazamycin B, Caprazamycin C, Caprazamycin E, Caprazamycin F, Capromorelin, Carafiban Maleate, Carbachol, Carbamazepine, Carbetocin, Carbovir, Carboxyamidotriazole, Cariporide Hydrochloride, Carisbamate, Carpipramine, Carumonam Sodium, Caspofungin Acetate, Cefaclor, Cefcanel Daloxate Hydrochloride, Cefcapene Pivoxil Hydrochloride, Cefdaloxime, Cefdaloxime Pentexil Tosilate, Cefdinir, Cefditoren Pivoxil, Cefepime, Cefetamet Pivoxil, Cefetecol, Cefixime, Cefluprenam, Cefmatilen Hydrochloride Hydrate, Cefmenoxime Hydrochloride, Cefminox Sodium, Cefodizime, Cefodizime Sodium, Cefoselis Sulfate, Cefotaxime Sodium, Cefotetan Disodium, Cefotiam Hexetil, Cefotiam Hexetil Hydrochloride, Cefotiam Hydrochloride, Cefoxitin, Cefozopran, Cefozopran Hydrochloride, Cefpirome, Cefpodoxime Proxetil, Cefprozil, Cefprozil Monohydrate, Cefquinome, Ceftaroline, Ceftazidime, Cefteram Pivoxil, Ceftibuten, Ceftobiprole, Ceftobiprole Medorcaril, Ceftrazonal Bopentil, Ceftrazonal Sodium, Ceftriaxone Sodium, Ceftrizoxime Alapivoxil, Cefuroxime, Cefuroxime Axetil, Cefuroxime Pivoxetil, Centanamycin, Cephalexin Monohydrate, Ceranapril, Ceruletide Diethylamine, Cetefloxacin, Chlorofusin, Chloroorienticin A, Chloroorienticin B, Chlorotetain, Cibrostatin 1, Cidofovir, Cilastatin Sodium, Cilengitide, Cimaterol, Cinitapride Hydrogen Tartrate, Cipamfylline, Circinamide, Cisapride Hydrate, Cispentacin, Citicoline, Citrullimycine A, Cladribine, Clitocine, Clofarabine, Clopidogrel Sulfate, Compound 301029, Coumamidine Gamma1, Coumamidine Gamma2, Cromoglycate Lisetil Hydrochloride, Cycallene, Cyclic-Cidofovir, Cycloserine, Cyclotheonamide A, Cyclothialidine, Cygalovir, Cypemycin, Cysmethynil, Cystamidin A, Cystamine, Cystazosin, Cystocin, Cytarabine, Cytarabine Ocfosfate, Cytaramycin, Cytochlor, Cytomodulin, Dabigatran, Dabigatran Etexilate, Dacopafant, Dactimicin, Dactinomycin, Dactylocycline A, Dactylocycline B, DADME-Immucillin-G, Dalargin, Danegaptide Hydrochloride, Dapropterin Dihydrochloride, Dapsone, Darbufelone Mesilate, Darifenacin Hydrobromide, Darinaparsin, Darunavir, Daunorubicin, Davasaicin, Davunetide, Debrisoquine Sulfate, Decahydromoenomycin A, Decaplanin, Deferoxamine, Degarelix Acetate, Delafloxacin, Delta-Aminolevulinic Acid Hydrochloride, Deltibant, Denagliptin Hydrochloride, Denibulin Hydrochloride, Denufosol Tetrasodium, Deoxymethylspergualin, Deoxynegamycin, Deoxyvariolin B, Desacetylvinblastinehydrazide/Folate Conjugate, Des-F-Sitagliptin, Desglugastrin Tromethamine, Deslorelin, Desmopressin Acetate, Detiviciclovir Diacetate, Dexelvucitabine, Dexibuprofen Lysine, Dextroamphetamine Sulfate, Dezinamide, Dezocitidine, Diadenosine Tetraphosphate, Diaveridine, Dichlorobenzoprim, Dicloguamine Maleate, Didemnin X, Didemnin Y, Dideoxycytidine, Difurazone, Dilevalol, Dilevalol Hydrochloride, Disermolide, Disopyramide Phosphate, DI-VAL-L-DC, Docosyl Cidofovir, Dolastatin 14, Dolastatin C, Donitriptan Hydrochloride, Donitriptan Mesilate, Dovitinib Lactate, Doxazosin Mesylate, Doxorubicin Hydrochloride, Doxycycline Hyclate, D-Penicillamine, Draflazine, Droxidopa, DTPA-Adenosylcobalamin, Ebrotidine, Ecenofloxacin Hydrochloride, Efegatran Sulfate Hydrate, Eflornithine Hydrochloride, Eglumegad Hydrate, Eicosyl Cidofovir, Elacytarabine, Elastatinal B, Elastatinal C, Elpetrigine, Elvucitabine, Emtricitabine, Enalkiren, Enigmol, Eniporide Mesilate, Entecavir, Entinostat, Epinastine Hydrochloride, Epiroprim, Epirubicin Hydrochloride, Epithalon, Epofolate, Epostatin, Epsilon Aminocaproic Acid, Eremomycin, Eribulin Mesylate, Erucamide, Esafloxacine Hydrochloride, Eslicarbazepine Acetate, Etaquine, Ethanolamine, Ethylthio-DADME-Immucillin-A, Ethynylcytidine, Etravirine, Etriciguat, Exalamide, Examorelin, Exatecan Mesilate, Ezatiostat Hydrochloride, Famciclovir, Famotidine, Famotidine Bismuth Citrate, Favipiravir, Feglymycin, Felbamate, Fenleuton, Fidarestat, Fidexaban, Filaminast, Filarizone, Fingolimod Hydrochloride, Flucytosine, Fludarabine Phosphate, Fluorobenzyltriamterene, Fluorominoxidil, Fluoroneplanocin A, Flupiritine Maleate, Fluvirucin B2, Fluvoxamine Maleate, Folinic Acid, Fortimicin A, Fosamprenavir Calcium, Fosamprenavir Sodium, Fosfomycin Trometamol, Fradafiban, Freselestat, Frovatriptan, Fudosteine, Furamidine, G1 Peptide, Gabadur, Gabapentin, Gabexate Mesilate, Galarubicin Hydrochloride, Galmic, Galnon, Ganciclovir, Ganciclovir Elaidic Acid, Ganciclovir Monophosphate, Ganciclovir Sodium, Ganirelix, Ganirelix Acetate, Garomefrine Hydrochloride, Gemcitabine, Gemcitabine Elaidate, Gemifloxacin Mesilate, Gilatide, Girodazole, Glaspimod, Glucosamine Sulfate, Gludopa, Glutathione Monoethylester, Glutathione Monoisopropylester, Glycine-Proline-Melphalan, Glycopin, Glycothiohexide alpha, Golotimod, Goserelin, Growth Factor Antagonist-116, Growth Hormone Releasing Peptid 2, Guanabenz Acetate, Guanadrel Sulfate, Guanethidine Monosulfate, Guanfacine Hydrochloride, Gusperimus Hydrochloride, Halovir A, Halovir B, Halovir C, Halovir D, Halovir E, Hayumicin B, Hayumicin C1, Hayumicin C2, Hayumicin D, Helvecardin A, Helvecardin B, Hepavir B, Heptaminol AMP Amidate, Hexa-D-Arginine, Hexadecyl Cidofovir, Hexadecyloxypropyl-Cidofovir, Histamine Dihydrochloride, Histaprodifen, Histrelin, Histrelin Acetate, Human Angiotensin II, Hydrostatin A, Hydroxyakalone, Hydroxyurea, Hypeptin, Ibutamoren Mesilate, Icatibant Acetate, Iclaprim, Icofungipen, Idarubicin Hydrochloride, Ilatreotide, Ilonidap, Imetit, Imidafenacin, Imidazenil, Imiquimod, Immunosine, Impentamine, Incyclinide, Indanocine, Indantadol Hydrochloride, Indoxam, Inogatran, Intrifiban, Iobenguane[131I], Iodorubidazone (P), Iotriside, Isepamicin Sulfate, Isobatzelline A, Isobatzelline B, Isobatzelline C, Isobatzelline D, Isobutyramide, Isodoxorubicin, Isopropamide Iodide, Ispinesib Mesylate, Istaroxime, Janthinomycin A, Janthinomycin B, Janthinomycin C, Jaspine B, Kahalalide F, Kaitocephalin, Kanamycin, Karnamicin B1, Katanosin A, Katanosin B, Kistamicin A, L-4-Oxalysine, Labetalol Hydrochloride, Labradimil, Lagatide, Lamifiban, Lamivudine, Lamotrigine, Lanicemine 2(S)-Hydroxysuccinate, Lanicemine Hydrochloride, Lanomycin, Larazotide Acetate, Lazabemide Hydrochloride, L-Dopa Methyl Ester Hydrochloride, L-Dopamide, Lecirelin, Lenalidomide, Lenampicillin Hydrochloride, Leucettamine A, Leucovorin Calcium, Leuprolide Acetate, Leurubicin, Leustroducsin A, Leustroducsin B, Leustroducsin C, Leustroducsin H, Levetiracetam, Levodopa, Levodopa 3-O-Glucoside, Levodopa 4-O-Glucoside, Levoleucovorin Calcium, L-Histidinol, L-Homothiocitrulline, Liblomycin, Linagliptin, Linifanib, Lintopride, Lirexapride, Lirimilast, Lisinopril, L-Lysine-D-Amphetamine Dimesylate, Lobophorin A, Lobucavir, Lodenosine, Loloatin B, Lomeguatrib, Lometrexol, Lonafarnib, Loracarbef Hydrate, Loviride, Loxoribine, L-Simexonyl Homocysteine, L-Thiocitrulline, Lymphostin, Lysobactin, Mabuterol Hydrochloride, Makaluvamine A, Makaluvamine A, Makaluvamine B, Makaluvamine C, Managlinat Dialanetil, Matristatin A2, Melagatran, Melanotan II, Memantine Hydrochloride, Memno-Peptide A, Meprobamate, Meriolin-3, Mersacidin, Metaraminol, Metazosin, Metformin Hydrochloride, Methotrexate, Methyl Bestatin, Methyldopa, Methylthio-DADME-Immucillin-A, Metoclopramide Hydrochloride, Metyrosine, Mexiletine Hydrochloride, Micafungin Sodium, Midaxifylline, Mideplanin, Midoriamin, Milacainide Tartrate, Milacemide-[2H], Milnacipran Hydrochloride, Minamestane, Minocycline Hydrochloride, Minoxidil, Mirabegron, Mitomycin, Mivazerol, Mivobulin Isethionate, Mizoribine, Mocetinostat Dihydrobromide, Modafinil, Modafinil Sulfone, Moenomycin A Chloride Bismuth Salt, Mofegiline, Mofegiline Hydrochloride, Monamidocin, Monodansyl Cadaverine, Montirelin Tetrahydrate, Mosapride Citrate, Moxilubant, Moxilubant Maleate, Mozenavir Mesilate, M-Phenylene Ethynylene, Muraminomicin A, Muraminomicin B, Muraminomicin C, Muraminomicin D, Muraminomicin E1, Muraminomicin E2, Muraminomicin F, Muraminomicin G, Muraminomicin H, Muraminomicin I, Muraminomicin Z1, Muraminomicin Z2, Muraminomicin Z3, Muraminomicin Z4, Muramyl Dipeptide C, Mureidomycin A, Mureidomycin B, Mureidomycin C, Mureidomycin D, Mycestericin E, Myriocin, Nafamostat Mesylate, Nafarelin Acetate, Naglivan, Namitecan, Napsagatran, Nebostinel, Nebracetam Fumarate, Neldazosin, Nelzarabine, Nemonoxacin, Neomycin B-Hexaarginine Conjugate, Neomycin-Acridine, Nepafenac, Nepicastat Hydrochloride, Neramexane Hydrochloride, Neridronic Acid, Netamiftide Trifluoroacetate, Netilmicin Sulfate, Nocathiacin I, Nocathiacin II, Nocathiacin III, Nocathiacin IV, NO-Gabapentin, Nolatrexed Hydrochloride, NO-Mesalamine, Noraristeromycin, Nuvanil, O6-Benzylguanine, Ocimumoside A, Octacosamicin A, Octacosamicin B, Octreother, Octreotide Acetate, Oglufanide Disodium, Olamufloxacin, Olamufloxacin Mesilate, Olcegepant, Olradipine Hydrochloride, Omaciclovir, Ombrabulin, Ombrabulin Hydrochloride, Onnamide A, Opiorphin, Orbofiban Acetate, Orienticin A, Orienticin B, Orienticin C, Orienticin D, Oritavancin, Oseltamivir Carboxylate, Oseltamivir Phosphate, Otamixaban, Otenabant Hydrochloride, Ovothiol A, Oxazofurin, Oxcarbazepine, Oxiglutatione Sodium, Oxiracetam, Oxolide, Oxynor, Oxyphenarsine, Ozarelix, Pachymedusa Dacnicolor Tryptophyllin-1, Paecilaminol, Pafuramidine Maleate, PalauÀmine, Paldimycin B, Pamidronate Sodium, Pancopride, Papuamide A, Papuamide B, Papuamide C, Papuamide D, Parasin I, Paromomycin, Pasireotide, Paulomycin, Paulomycin A2, Paulomycin B, Paulomycin C, Paulomycin D, Paulomycin E, Paulomycin F, Pazufloxacin, Pazufloxacin Mesilate, PEG-Vancomycin, Pelagiomicin C, Peldesine, Pelitrexol, Pemetrexed Disodium, Penciclovir, Penicillin G Procaine, Pentamidine Gluconate, Pentamidine Isethionate, Pentamidine Lactate, Peplomycin, Peramivir, Perphanazine 4-Aminobutyrate, Phakellistatin 5, PHE-ARG-Beta-Naphthylamide, Phentermine, Phortress, Phospholine, Pibutidine Hydrochloride, Pimeloylanilide O-Aminoanilide, Piracetam, Pirarubicin, Pivampicillin, Pixantrone Maleate, Pluraflavin A, Pluraflavin B, Plusbacin A1, Plusbacin A2, Plusbacin A3, Plusbacin A4, Plusbacin B1, Plusbacin B2, Plusbacin B3, Plusbacin B4, PMEO-5-ME-DAPY, Pneumocandin A0, Pneumocandin B0, Pneumocandin B0 2-Phosphate, Pneumocandin D0, Polaprezinc, Polydiscamide A, Polymer Bound Human Leukocyte Elastase Inhibitor, Poststatin, PPI17-24, Pradimicin E, Pradimicin FA-2, Pralatrexate, Pramipexole Hydrochloride, Pranedipine Tartrate, Prazosin Hydrochloride, Prefolic A, Pregabalin, Preladenant, Primaquine Phosphate, Probestin, Procainamide Hydrochloride, Procaine Hydrochloride, Pro-Diazepam, Prostatin, Prucalopride, Prucalopride Hydrochloride, Prucalopride Succinate, Pseudomycin A′, Pseudomycin B′, Pyloricidin B, Pyradizomycin, Pyrazinamide, Pyrazinoylguanidine, Pyriferone, Pyrimethamine, Quinelorane Hydrochloride, R-(+)-Aminoindane, Ralfinamide, Ramoplanin A′1, Ramoplanin A′2, Ramoplanin A′3, Ramorelix, Ravidomycin N-oxide, Razaxaban Hydrochloride, Reblastatin, Regadenoson, Relcovaptan, Remacemide Hydrochloride, Resiquimod, Restricticin, Retaspimycin Hydrochloride, Retigabine Hydrochloride, Rhodopeptin C1, Rhodopeptin C2, Rhodopeptin C3, Rhodopeptin C4, Rhodostreptomycin A, Rhodostreptomycin B, Ribavirin, Ribavirin Eicosenate cis, Ribavirin Eicosenate trans, Ribavirin Elaidate, Ribavirin Oleate, Rilmazafone Hydrochloride Dihydrate, Riluzole, Rimacalib Hydrochloride, Rimeporide Hydrochloride, Riociguat, Ritipenem Acoxil, Robalzotan Hydrochloride, Robalzotan Tartrate Hydrate, Rociclovir, Romurtide, Rotigaptide, Roxifiban Acetate, Ruboxyl, Rufinamide, Rumycin 1, Rumycin 2, Sabarubicin Hydrochloride, Sabiporide Mesilate, Safinamide Mesilate, Safingol, Sagamacin, Sampatrilat, Sampirtine, Saprisartan, Saquinavir, Saquinavir Mesilate, Sardomizide Hydrochloride, Sardomozide, Saussureamine C, Saxagliptin, Secobatzelline A, Secobatzelline B, Seglitide, Selank, Seletracetam, Semapimod Hydrochloride, Senicapoc, Sepimostat Mesilate, Seproxetine, Seraspenide, Sevelamer Carbonate, Sevelamer Hydrochloride, Shepherdin, Sibrafiban, Silodosin, Silver Sulfadiazine, Sipatrigine, Sitafloxacin Hydrate, Sitagliptin Phosphate Monohydrate, S-Nitrosoglutathione, Sofigatran, Sonedenoson, Sotirimod, Sparfloxacin, Sperabillin A, Sperabillin B, Sperabillin C, Sperabillin D, Sphingofungin F, Spinorphin, Spisulosine, Squalamine Lactate, Streptomycin, Styloguanidine, Substance P(8-11), Sufinosine, Sulcephalosporin, Sulfostin, Sulphazocine, Sultamicilline Tosylate, Sunflower Trypsin Inhibitor-1, Surfen, Synadenol, Synguanol, Tabimorelin, Tacedinaline, Tacrine Hydrochloride, Tageflar, Talabostat, Talaglumetad Hydrochloride, Talampanel, Talipexole Dihydrochloride, Tallimustine Hydrochloride, Talopterin, Taltirelin, Tanespimycin, Tanogitran, Targinine, Technetium (99MTC) Depreotide, Teicoplanin-A2-1, Teicoplanin-A2-2, Teicoplanin-A2-3, Teicoplanin-A2-3, Teicoplanin-A2-5, Telavancin Hydrochloride, Telinavir, Temozolomide, Temurtide, Tenidap, Tenidap Sodium, Tenofovir, Tenofovir DF, Terazosin Hydrochloride, Tetracosyl Cidofovir, Tetracycline Hydrochloride, Tetrafibricin, Texenomycin A, Tezacitabine, TGP, Thioacet, Thiothio, Thrazarine, Thymoctonan, Thymopentin, Tiamdipine, Tigecycline, Tilarginine Hydrochloride, Timirdine Diethanesulfonate, Timodepressin, Tipifarnib, TNF-Alpha Protease Enzyme Inhibitor, Tobramycin, Tocainide Hydrochloride, Tokaramide A, Tomopenem, Topostatin, Torcitabine, Tosufloxacin, Tosufloxacin Tosilate, Tranexamic Acid, Trantinterol Hydrochloride, Tranylcypromine Sulfate, Trelanserin, Tresperimus Triflutate, Trichomycin A, Triciribine, Triciribine Phosphate, Trientine Hydrochloride, Trimazosin Hydrochloride, Trimetrexate Glucuronate, Trimexautide, Trimidox, Trovafloxacin, Trovafloxacin Hydrate, Trovafloxacin Hydrochloride Mesylate, Trovafloxacin Mesilate, Troxacitabine, Trybizine Hydrochloride, Tubastrine, Tuftsin, Tyroservatide, Tyrphostin 47, Ubenimex, Valacyclovir, Valganciclovir Hydrochloride, Valnemulin, Valomaciclovir Stearate, Valonomycin A, Valopicitabine, Valpromide, Valrocemide, Vamicamide, Vancomycin Hydrochloride, Vancoresmycin, Vapitadine Hydrochloride, Varespladib, Varespladib Methyl, Varespladib Mofetil, Velnacrine Maleate, Venorphin, Vigabatrin, Vilazodone Hydrochloride, Vindesine, Viramidine Hydrochloride, Viranamycin-B, Vitamin B3, W Peptide, Xemilofiban, Xylocydine, Zanamivir, Zileuton, Zoniporide Hydrochloride, Zorubicin Hydrochloride, ACTH, adenosine deaminase, agalsidase, albumin, alfa-1 antitrypsin (AAT), alfa-1 proteinase inhibitor (API), alglucosidase, alteplase, anistreplase, ancrod serine protease, antibodies (monoclonal or polyclonal and fragments or fusions), antithrombin III, antitrypsins, aprotinin, asparaginases, biphalin, bone-morphogenic proteins, calcitonin (salmon), collagenase, DNase, endorphins, enfuvirtide, enkephalins, erythropoietins, factor VIIa, factor VIII, factor VIIIa, factor IX, fibrinolysin, fusion proteins, follicle-stimulating hormones, granulocyte colony stimulating factor (G-CSF), galactosidase, glucagon, glucagon-like peptides like GLP-1, glucocerebrosidase, granulocyte macrophage colony stimulating factor (GM-CSF), chorionic gonadotropin (hCG), hemoglobins, hepatitis B vaccines, hirudin, hyaluronidases, idurnonidase, immune globulins, influenza vaccines, interleukines (1 alfa, 1 beta, 2, 3, 4, 6, 10, 11, 12), IL-1 receptor antagonist (rhIL-1ra), insulins, interferons (alfa 2a, alfa 2b, alfa 2c, beta 1a, beta 1b, gamma 1a, gamma 1b), keratinocyte growth factor (KGF), lactase, leuprolide, levothyroxine, luteinizing hormone, lyme vaccine, natriuretic peptide, pancrelipase, papain, parathyroid hormone, PDGF, pepsin, phospholipase-activating protein (PLAP), platelet activating factor alcetylhydrolase (PAF-AH), prolactin, protein C, octreotide, secretin, sermorelin, superoxide dismutase (SOD), somatropins (growth hormone), somatostatin, streptokinase, sucrase, tetanus toxin fragment, tilactase, thrombins, thymosin, thyroid stimulating hormone, thyrothropin, transforming growth factors, tumor necrosis factor (TNF), TNF receptor-IgG Fc, tissue plasminogen activator (tPA), transferrin, TSH, urate oxidase, urokinase, Fab (fragment, antigen-binding), F(ab)2 fragments, Fc (fragment, crystallizable), pFc' fragment, Fv (fragment, variable), scFv (single-chain variable fragment), di-scFv/diabodies, bi-specific T-cell engager, CDRs (complementarity determining regions), single-domain antibodies (sdABs/Nanobodies), heavy chains (α, δ, ε, γ, μ) or heavy chain fragments, light chains (λ, κ) or light chain fragments, VH fragments (variable region of the heavy chain), VL fragments (variable region of the light chain), VHH fragments, VNAR fragments, shark-derived antibody fragments and affinity scaffold proteins, Kunitz domain-derived affinity scaffold proteins, centyrin-derived affinity scaffold proteins, ubiquitin-derived affinity scaffold proteins, lipocalin-derived affinity scaffold proteins, ankyrin-derived affinity scaffold proteins, Versabodies (disulfide-rich affinity scaffold proteins), fibronectin-derived affinity scaffold proteins, cameloid-derived antibody fragments and affinity scaffold proteins, llama-derived antibody fragments and affinity scaffold proteins, transferrin-derived affinity scaffold proteins, Squash-type protease inhibitors with cysteine-knot scaffold-derived affinity scaffold proteins.

Suitable secondary amine-containing drugs may be selected from the group consisting of (−)-3-O-Acetylspectaline hydrochloride, (−)-3-O-tert-Boc-spectaline hydrochloride, (−)-Cicloprolol, (−)-Norchloro-[18F]fluoro-homoepibatidine, (−)-Salbutamol hydrochloride, (−)-Salmeterol, (+)-(S)-Hydroxychloroquine, (+)-Isamoltan, (+)-R-Pramipexole, (R)-(+)-Amlodipine, (R)-Clevidipine, (R)-NSP-307, (R)-Teludipine, (R)-Thionisoxetine, (S)-Clevidipine, (S)—N— Desmethyltrimebutine, (S)-Noremopamil, [99Tc]Demobesin 4, [Glu10,Nle17,Nle30]-Pancreatic polypeptide(2-36), [Nle17,Nle30]-Pancreatic polypeptide(2-36), [psi[CH2NH]Tpg4]Vancomycin aglycon, 15bbeta-Methoxyardeemin, 3-Bromomethcathinone, 4,5-Dianilinophthalimide, 4-Hydroxyatomoxetine, 5-Methylurapidil, 7-Oxostaurosporine, 99mTc-c(RGDfK*)2HYNIC, A-42867 pseudoaglycone, Abacavir succinate, Abacavir sulfate, Abarelix, Acarbose, Acebutolol hydrochloride, Aceclofenac, Acyline, Adaphostin, Adaprolol maleate, Adaprolol oxalate, Adecypenol, Adrogolide hydrochloride, Aglaiastatin C, Alchemix, Alinidine, Alkasar-18, Alminoprofen, Alniditan, alpha-Methylepinephrine, Alprafenone hydrochloride, Alprenolol hydrochloride, Alprenoxime hydrochloride, Altromycin A, Altromycin C, Alvespimycin hydrochloride, Ambroxol nitrate, Amfebutamone hydrochloride, Amibegron hydrochloride, Amifostine hydrate, Amineptine, Aminocandin, Aminochinol, Amitivir, Amlodipine, Amlodipine besylate, Amocarzine, Amodiaquine, Amosulalol hydrochloride, Amoxapine, Amsacrine, Anabasine hydrochloride, Anisperimus, Antide-1, Aranidipine, Araprofen, Arbutamine hydrochloride, Ardeemin, Arformoterol tartrate, Argatroban monohydrate, Argiopine, Arotinolol hydrochloride, Asperlicin E, Atenolol, Atevirdine mesylate, Azathioprine, Azelnidipine, Azepinostatin, Balamapimod, Balhimycin, Balofloxacin, Balofloxacin dihydrate, Bambuterol, Bamirastine hydrate, Banoxantrone, Baogongteng A, Barixibat, Barnidipine hydrochloride, Batoprazine, Batzelline A, Batzelline B, Batzelline C, Becampanel, Bederocin, Bedoradrine sulfate, Befunolol hydrochloride, Belactin B, Belotecan hydrochloride, Benazepril hydrochloride, Bendroflumethiazide, Benidipine hydrochloride, Berlafenone hydrochloride, Betaxolol hydrochloride, Bevantolol hydrochloride, Biemnidin, Bifemelane hydrochloride, Binospirone mesylate, Bioxalomycin alpha 1, Bis(7)-cognitin, Bisantrene hydrochloride, Bisnafide mesilate, Bisoprolol fumarate, Bitolterol mesylate, Bleomycin A2 sulfate, Boholmycin, Bopindolol, Bosutinib, Brinazarone, Brinzolamide, Bulaquine, Bumetanide, Buteranol, Butofilolol, Cadrofloxacin hydrochloride, Caldaret hydrate, Calindol Dihydrochloride, Capridine beta, Carmoterol hydrochloride, Carteolol hydrochloride, Carvedilol, Caspofungin acetate, Ceftaroline fosamil acetate, Ceftizoxime sodium, Ceftobiprole, Celiprolol hydrochloride, Cerebrocrast, Ceruletide diethylamine, Cevipabulin, Chinoin-169, Chloptosin, Chlordiazepoxide hydrochloride, Chloroorienticin A, Chloroorienticin B, Cilazapril, Cilnidipine, Ciluprevir, Cimaterol, Cinacalcet hydrochloride, Cinnamycin, Ciprofloxacin hydrochloride, Ciprofloxacin silver salt, Clevidipine butyrate, Clitocine, Clopenphendioxan, Cloranolol hydrochloride, Clozapine, Conantokin-R, Conophylline, Crisnatol mesilate, Cronidipine, Dabelotine mesilate, Dabigatran, Dabigatran etexilate, Dalbavancin, Dapivirine, Dapropterin dihydrochloride, Dasantafil, Debromoshermilamine, Decaplanin, Degarelix acetate, Delapril hydrochloride, Delavirdine mesilate, Delfaprazine hydrochloride, Delucemine hydrochloride, Demethylallosamidin, Demexiptiline hydrochloride, Denopamine, Deoxymethylspergualin, Deoxyspergualin Hydrochloride, Desacetylvinblastinehydrazide/folate conjugate, Desbutyl benflumetol, Desbutylhalofantrine hydrochloride, Desferri-salmycin A, Desferri-salmycin B, Desferri-salmycin C, Desferri-salmycin D, Desipramine hydrochloride, Desloratadine, Dexfenfluramine hydrochloride, Dexketoprofen meglumine, Dexmethylphenidate hydrochloride, Dexniguldipine hydrochloride, Dexsotalol, Diazepinomicin, Dichlorobenzoprim, Diclofenac potassium, Diclofenac sodium, Diclofenac zinc salt, Diethylnorspermine, Dihydrexidine, Dilevalol, Dilevalol hydrochloride, Dinapsoline, Dinoxyline, Dipivefrine hydrochloride, Discodermide, Discodermide acetate, Discorhabdin D, Discorhabdin P, Discorhabdin S, Discorhabdin T, Discorhabdin U, Dobutamine hydrochloride, Dobutamine phosphate, Dopexamine, Dopexamine hydrochloride, Doripenem, Dorzolamide hydrochloride, d-Pseudoephedrine hydrochloride, Droxinavir, Duloxetine hydrochloride, Duocarmycin A, Duocarmycin B1, Duocarmycin B2, Duocarmycin C1, Duocarmycin C2, Dynemicin A, Dynemicin C, Ebanicline, Ecteinascidin 1560, Ecteinascidin 722, Ecteinascidin 729, Ecteinascidin 736, Ecteinascidin 745, Ecteinascidin 770, Ecteinascidin 875, Efaroxan, Efegatran sulfate hydrate, Efepristin, Efonidipine hydrochloride ethanol, Elagolix sodium, Elansolid C1, Elarofiban, Elbanizine, Elgodipine hydrochloride, Elinafide mesilate, Elinogrel potassium, Elnadipine, Enalapril maleate, Enalapril nitrate, Enalaprilat, Enazadrem, Enkastin (D), Enkastin (D), Enkastin (D), Enkastin AD, Enkastin AE, Enkastin ID, Enkastin IE, Enkastin VD, Enkastin VE, Enoxacin, Epibatidine, Epostatin, Eremomycin, Ersentilide, Ersentilide hydrochloride, Ertapenem sodium, Esculeogenin A, Esculeoside A, Esmolol hydrochloride, Esperamicin A1, Etamsylate, Ethoxy-idazoxan, Eugenodilol, Ezlopitant, Falnidamol, Farglitazar, Fasobegron hydrochloride, Fasudil hydrochloride, Felodipine, Fenoldopam mesilate, Fenoterol hydrobromide, Fepradinol, Ferroquine, Ferulinolol, Finafloxacin hydrochloride, Flecainide acetate, Florbetaben, Florbetapir F 18, Flufenoxine, Flumezapine, Fluodipine, Fluoxetine hydrochloride, Fluparoxan, Flupirtine maleate, Foetidine 1, Foetidine 2, Folinic acid, Formoterol fumarate, Forodesine hydrochloride, Fosaprepitant dimeglumine, Fosopamine, Frovatriptan, Furnidipine, Furosemide, Gaboxadol, Gadobenic acid dimeglumine salt, Gadopentetate dimeglumine, Gadoterate meglumine, Galactomycin I, Galactomycin II, Garenoxacin mesilate, Gatifloxacin, Gefitinib, Glucolanomycin, Glutapyrone, Gosogliptin hydrochloride, Grepafloxacin hydrochloride, Gypsetin, Halofuginone hydrobromide, Helvecardin A, Helvecardin B, Herquline B, Hesperadin, Himastatin, Hispidospermidin, Homoepibatidine, Hydrochlorothiazide, Hydroflumethiazide, Hydroxychloroquine sulfate, Ibopamine, Idazoxan hydrochloride, Iganidipine hydrochloride, Imidapril, Imidapril hydrochloride, Imidazoacridinone, Imisopasem manganese, Immepip, Immepyr, Incadronate, Indacaterol, Indantadol hydrochloride, Indeloxazine hydrochloride, Indolmycin, Inogatran, Intoplicine, Iofetamine hydrochloride I-123, Iptakalim hydrochloride, Isavuconazonium chloride hydrochloride, Isepamicin sulfate, Isofagomine tartrate, Isoquine, Ispronicline, Isradipine, Iturelix, Kaitocephalin, Ketamine hydrochloride, Kopsinine, Korupensamine A, Korupensamine B, Korupensamine C, Kosinostatin, Labedipinedilol A, Labedipinedilol B, Labetalol hydrochloride, Labradimil, Lacidipine, Ladasten, Ladostigil tartrate, Lagatide, Landiolol, Lapatinib ditosylate, Lenapenem hydrochloride, Lenapenem hydrochloride hydrate, Lerisetron, Leucovorin calcium, Levobetaxolol hydrochloride, Levobunolol hydrochloride, Levoleucovorin calcium, Levonebivolol, Liblomycin, Linaprazan, Lisinopril, Litoxetine, Lobenzarit sodium, Lodamin, Lofexidine hydrochloride, Lomefloxacin hydrochloride, Lorcaserin, Lotrafiban, Loviride, Lubazodone hydrochloride, Lumiracoxib, Mabuterol hydrochloride, Makaluvamine D, Makaluvamine E, Makaluvamine F, Makaluvone, Manidipine hydrochloride, Manifaxine hydrochloride, Manzamine B, Manzamine D, Maprotiline hydrochloride, Maropitant, Masnidipine hydrochloride, Mecamylamine hydrochloride, Meclofenamate sodium, Mefenamic acid, Mefloquine hydrochloride, Melagatran, Melogliptin, Meluadrine, Meluadrine tartrate, Memoquin, Mepindolol sulfate, Mepindolol transdermal patch, Meropenem, Methamphetamine hydrochloride, Methoctramine, Methyclothiazide, Methylhistaprodifen, Methylphenidate hydrochloride, Metipranolol, Metolazone, Metoprolol fumarate, Metoprolol succinate, Metoprolol tartrate, Mezacopride, Michellamine B, Microcin J25, Micronomicin sulfate, Midafotel, Milacemide-[2H], Minaprine hydrochloride, Mirabegron, Mitomycin, Mitoxantrone hydrochloride, Mivobulin isethionate, Modipafant, Moexipril hydrochloride, Moexiprilat, Montirelin tetrahydrate, Moranolin, Motesanib diphosphate, Moxifloxacin hydrochloride, Moxonidine hydrochloride hydrate, Muraminomicin I, Mureidomycin E, Mureidomycin F, Mureidomycins, N1,N8-Bisnorcymserine, Nadolol, Naproxen piperazine, Napsamycin A, Napsamycin B, Napsamycin C, Napsamycin D, Nardeterol, N-demethylated sildenafil, Nebivolol, Nemonapride, Neomycin-acridine, Neratinib, Netilmicin sulfate, Nicardipine hydrochloride, Nifedipine, Nifekalant hydrochloride, Niguldipine hydrochloride, Nilvadipine, Nimodipine, Nipradilol, Nisoldipine, Nitracrine dihydrochloride hydrate, Nitrendipine, Nitrofenac, Nitroso-nifedipine, Noberastine, Noberastine citrate, NO-ciprofloxacin, N-Octyl-beta-valienamine, Nolomirole hydrochloride, Norfloxacin, Norsegoline, Nortopixantrone hydrochloride, Nortriptyline hydrochloride, N-tert butyl isoquine, Oberadilol, Oberadilol monoethyl maleate, Odanacatib, Olanzapine, Olanzapine pamoate, Olradipine hydrochloride, Ontazolast, OPC-17083, Orbifloxacin, Orciprenaline sulphate, Orienticin A, Orienticin B, Orienticin C, Oritavancin, Osemozotan hydrochloride, Osutidine, Otenabant hydrochloride, Ovothiol B, Oxprenolol hydrochloride, Ozenoxacin, Pafenolol, Palau'amine, Palindore fumarate, Panobinostat, Parodilol hemifumarate, Parogrelil hydrochloride, Paroxetine, Paroxetine ascorbate, Paroxetine camsilate, Paroxetine hydrochloride, Paroxetine mesilate, Pazelliptine trihydrochloride, Pazelliptine trihydrochloride monohydrate, Pelitinib, Pelitrexol, Penbutolol sulfate, Pentostatin, Peplomycin, Perindopril, Perzinfotel, Phendioxan, Pibutidine hydrochloride, Picumeterol fumarate, Pindolol, Pirbuterol hydrochloride, Pittsburgh Compound B, Pixantrone maleate, Plerixafor hydrochloride, Polyglutamate camptothecin, Pozanicline hydrochloride, Pradimicin A, Pradimicin B, Pradimicin D, Pradimicin FA-1, Pradimicin FL, Pradimicin FS, Pradimicin L, Pradimicin S, Pradofloxacin, Pramipexole hydrochloride, Pranedipine tartrate, Pranidipine, Prefolic A, Premafloxacin, Premafloxacin hydrochloride, Premafloxacin magnesium, Primaquine phosphate, Prisotinol, Procaterol Hydrochloride Hemihydrate, Propafenone hydrochloride, Propranolol hydrochloride, Protriptyline hydrochloride, Proxodolol, Pumaprazole, Pyrindamycin A, Pyrindamycin B, Quinapril hydrochloride, Quinpramine, rac-Debromoflustramine E, Radezolid, Rafabegron, Ralfinamide, Ramipril, Rasagiline mesilate, Razupenem, Reboxetine mesilate, Repinotan, Repinotan hydrochloride, Reproterol hydrochloride, Retaspimycin hydrochloride, Retigabine hydrochloride, Rhodostreptomycin A, Rhodostreptomycin B, Rifabutin, Rilmenidine dihydrogen phosphate, Rimoterol hydrobromide, Risotilide, Rivanicline, Robenacoxib, Rolapitant hydrochloride, Safinamide mesilate, Sagandipine, Salbostatin, Salbutamol nitrate, Salbutamol sulfate, Salmaterol, Salmeterol xinafoate, Sarizotan hydrochloride, Saussureamine C, Sazetidine-A, Selodenoson, Sertraline, Sertraline hydrochloride, Setazindol, Sezolamide hydrochloride, Shishijimicin A, Shishijimicin B, Shishijimicin C, Sibanomicin, Sibenadet hydrochloride, Silodosin, Sitamaquine hydrochloride, Sivelestat sodium hydrate, Sofinicline, Solabegron hydrochloride, Solpecainol hydrochloride, Soraprazan, Sotalol hydrochloride, Sparfloxacin, Spermine dialdehyde, Spirapril, Spiroquinazoline, Squalamine lactate, Streptomycin, Stressinl-A, Sumanirole maleate, Suprofenac 1, Suprofenac 2, Suprofenac 3, Suronacrine maleate, Tafamidis meglumine, Tafenoquine succinate, Talarozole, Talibegron, Talibegron hydrochloride, Talniflumate, Talotrexin, Taltobulin, Taludipine hydrochloride, Tamsulosin hydrochloride, Tanespimycin, Tanogitran, Tauropyrone, Tazopsine, Tecalcet hydrochloride, Tecastemizole, Technetium (99mTc) apcitide, Technetium (99mTc) bicisate, Telatinib, Telavancin hydrochloride, Temacrazine mesilate, Temafloxacin hydrochloride, Temocapril hydrochloride, Terbutaline sulfate, Terodiline hydrochloride, Tertatolol hydrochloride, Tetracaine hydrochloride, Tetrahydrodercitin 1, Tetrindole, Tezampanel, Thiamet-G, Thiofedrine, Tiamdipine, Tiamenidine, Tianeptine sodium, Tiapafant, Tienoxolol hydrochloride, Tigecycline, Tilisolol hydrochloride, Timolol hemihydrate, Timolol maleate, Tinazoline hydrohloride, Tirofiban hydrochloride, Tizanidine hydrochloride, Toborinone, Tolfenamic acid, Tomatine, Tomoxetine hydrochloride, Topixantrone hydrochloride, Torasemide, Trabectedin, Trandolapril, Trandolaprilat, Trantinterol hydrochloride, Treprostinil diethanolamine, Tresperimus triflutate, Triacetyl dynemicin C, Trientine hydrochloride, Trifluproxim, Trimetazidine, Trimetrexate glucuronate, Trombodipine, Troxipide, Tulathromycin A, Tulathromycin B, Tulobuterol hydrochloride, Ufenamate, Ulifloxacin, Ulimorelin, Uncialamycin, Urapidil, Utibapril, Utibaprilat, Vabicaserin hydrochloride, Vancomycin hydrochloride, Vandetanib, Vanidipinedilol, Vaninolol, Vapitadine hydrochloride, Varenicline tartrate, Varlitinib, Vatalanib succinate, Vatanidipine, Vatanidipine hydrochloride, Vestipitant mesylate, Vicenistatin, Vildagliptin, Viloxazine hydrochloride, Vofopitant hydrochloride, Voglibose, Voreloxin, Xamoterol fumarate, Ximelagatran, Yttrium-90 edotreotide, Zabicipril hydrochloride, Zabiciprilat hydrochloride, Zabofloxacin hydrochloride, Zanapezil fumarate, Zelandopam hydrochloride, Zilpaterol, Zolmitriptan.

Suitable drugs containing aliphatic hydroxyl groups are, for example, (−)-(2R*,3R*,11bS*)- Dihydrotetrabenazine, (−)-(2R*,3S*, 11bR*)-Dihydrotetrabenazine, (−)-2-(2-Bromohexadecanoyl)paclitaxel, (−)-4′,5′-Didemethoxypicropodophyllin, (−)-4′-Demethoxypicropodophyllin, (−)-9-Dehydrogalanthaminium bromide, (−)-Calicheamicinone, (−)-Cicloprolol, (−)-Indocarbazostatin B, (−)-Kendomycin, (−)-Kolavenol, (−)-Salmeterol, (+)-(2R*,3R*, 11bS*)-Dihydrotetrabenazine, (+)-(2R*,3S*, 11bR*)-Dihydrotetrabenazine, (+)-(S)-Hydroxychloroquine, (+)-23,24-Dihydrodiscodermolide, (+)-Almuheptolide A, (+)-Azacalanolide A, (+)-Cystothiazole B, (+)-Dihydrocalanolide A, (+)-Etorphine, (+)-Hemipalmitoylcarnitinium, (+)-Indocarbazostatin, (+)-Isamoltan, (+)-SCH-351448, (+)-Sotalol, (E)-p-Coumaroylquinic acid, (R)-Almokalant, (R)-Bicalutamide, (R)-Dixyrazine dihydrochloride, (R)-Sulfinosine, (S)-Almokalant, (S)-Methylnaltrexone bromide, (S)-Oxiracetam, (S)-Sulfinosine, (Z)-Indenaprost, [125I]-Iodomethyllycaconitine, [8]-Gingerol, [Arg(Me)9] MS-10, [D-Tyr1,Arg(Me)9] MS-10, [D-Tyr1,AzaGly7,Arg(Me)9] MS-10, [D-Tyr1] MS-10, [N-MeIle4]-cyclosporin, [psi[CH2NH]Tpg4]Vancomycin aglycon, [Trp19] MS-10, 111In-Pentetreotide, 11-Hydroxyepothilone D, 11-Keto-Beta-Boswellic Acid, 12′-Methylthiovinblastine dihydrochloride, 13-Deoxyadriamycin hydrochloride, 14alpha-Lipoyl andrographolide, 14beta-Hydroxydocetaxel-1,14-acetonide, 14beta-Hydroxytaxotere, 14-C-Methyltriptolide, 14-Demethylmycoticin A, 14-Hydroxyclarithromycin, 14-Isobutanoylandrographolide, 14-Pivaloylandrographolide, 15-Methylepothilone B, 16-Methyloxazolomycin, 17-Aminogeldanamycin, 17beta-Hydroxywortmannin, 18,19-Dehydrobuprenorphine hydrochloride, 18-Hydroxycoronaridine, 19-O-Demethylscytophycin C, 19-O-Methylgeldanamycin, 1alpha,25-Dihydroxyvitamin D3-23,26-lactone, 1alpha-Hydroxyvitamin D4, 1-Oxorapamycin, 21-Aminoepothilone B, 22-Ene-25-oxavitamin D, 22-Oxacalcitriol, 24(S)-Ocotillol, 24-Deoxyascomycin, 25-Anhydrocimigenol-3-O-beta-D-xylopyranoside, 26-Fluoroepothilone, 2-Aminoaristeromycin, 2-Aminoneplanocin A, 2-Methoxyestradiol, 2′-Palmitoylpaclitaxel, 3,5-Dicaffeoylquinic acid, 3,7a-Diepialexine, 36-Dihydroisorolliniastatin 1, 3-Allyl farnesol, 3-Bromodiosmine, 3-Chlorodiosmine, 3-Deazaadenosine, 3-Epimaxacalcitol, 4,6-diene-Cer, 41-Demethylhomooligomycin B, 44-Homooligomycin B, 4-Chlorophenylthio-DADMe-immucillin-A, 4-Demethylepothilone B, 4′-Ethynylstavudine, 4″-Hydroxymevastatin lactone, 5(R)-Hydroxytriptolide, 5,4′-Diepiarbekacin, 5,6-Dehydroascomycin, 5′-Epiequisetin, 5-Ethylthioribose, 5-N-Acetyl-15balpha-hydroxyardeemin, 5-Phenylthioacyclouridine, 5-Thiaepothilone, 5Z-7-Oxozeaenol, 6alpha-7-Epipaclitaxel, 6alpha-Fluoroursodeoxycholic acid, 6′-Homoneplanocin A, 6-Hydroxyscytophycin B, 6-O-mPEG4-Nalbupine, 6-O-mPEG5-Nalbuphine, 7,7a-Diepialexine, 7-Deoxytaxol, 8(R)-Fluoroidarubicin hydrochloride, 9,11-Dehydrocortexolone 17alpha-butyrate, 9,9-Dihydrotaxol, 9-[18F]Fluoropropyl-(+)-dihydrotetrabenazine, 99mTc-c(RGDfK*)2HYNIC, 9-Aminocamptothecin, 9-Hydroxyrisperidone, A-42867 pseudoaglycone, Abacavir succinate, Abacavir sulfate, Abaperidone hydrochloride, Abarelix, Abietaquinone methide, Abiraterone, Acadesine, Acarbose, Acaterin, Acebutolol hydrochloride, Acemannan, Aceneuramic acid sodium salt, Achimillic Acids, Achimillicic Acid a Lactone, Aciclovir, Aclarubicin, Actinoplanone A, Actinoplanone B, Aculeacin Agamma, Acyline, Adamantyl globotriaosylceramide, Adaprolol maleate, Adaprolol Oxalate, Adecypenol, Adelmidrol, Ademetionine tosylate sulfate, Adenophostin A, Adenophostin B, Adenosine, Adlupulon, Adxanthromycin A, Aerothricin 1, Aerothricin 16, Aerothricin 41, Aerothricin 45, Aerothricin 5, Aerothricin 50, Aerothricin 55, Afeletecan hydrochloride, Agelasphin 517, Agelasphin 564, Aglaiastatin A, Aglaiastatin B, Aglaiastatin C, Aglepristone, Albaconazole, Albifylline, Albitiazolium bromide, Albocycline K3, Alclometasone dipropionate, Alcuronium chloride, Aldecalmycin, Alemcinal, Alendronate sodium, Alfacalcidol, Alisamycin, Aliskiren fumarate, Alkasar-18, Almokalant, alpha-C-Galactosylceramide, alpha-Galactosylceramide, alpha-Galactosylceramide-BODIPY, alpha-Lactosylceramide, alpha-Methylepinephrine, alpha-Methylnorepinephrine, Alprafenone hydrochloride, Alprenolol hydrochloride, Alprostadil, Altemicidin, Altorhyrtin C, Altromycin A, Altromycin B, Altromycin C, Altromycin D, Altromycins, Alvespimycin hydrochloride, Alvocidib hydrochloride, Amarogentin, Ambroxol nitrate, Amdoxovir, Amelometasone, Amibegron hydrochloride, Amikacin, Aminocandin, Ammocidin A, Amosulalol Hydrochloride, Amphidinolide E, Amphidinolide T1, Amphinidin A, Amphotericin B, Amprenavir, Amrubicin Hydrochloride, Amycolamicin, Amycomycin, Anandamide, Andenallene, ANDREA-1, Androstanolone, Androxolutamide, Anecortave acetate, Anguinomycin C, Anguinomycin D, Anidulafungin, Ankinomycin, Annamycin, Annocherimolin, Antheliatin, Antide, Antide-1, Antide-2, Antide-3, Antiflammin-1, Antiflammin-3, Apadenoson, Apaziquone, Aphidicolin, Aphidicolin Glycinate, Apicularen A, Apicularen B, Aplaviroc hydrochloride, Apricitabine, Aragusterol A, Aragusterol C, Aranorosin, Aranorosinol A, Aranorosinol B, Aranose, Arbekacin, Arbekacin sulfate, Arborcandin A, Arborcandin B, Arborcandin C, Arborcandin D, Arborcandin E, Arborcandin F, Arbutamine hydrochloride, Archazolid A, Archazolid B, Arformoterol tartrate, Arimoclomol maleate, Arisostatin A, Arisugacin A, Arotinolol hydrochloride, Artelinate, Arteminolide A, Arteminolide B, Arteminolide C, Arteminolide D, Artilide fumarate, Arundifungin, Ascosteroside, Asiatic acid, Asiaticoside, Asimadoline, Asperlicin B, Asperlicin E, Assamicin I, Assamicin II, Astromicin sulfate, Atazanavir sulfate, Atenolol, Atigliflozin, Atorvastatin, Atorvastatin calcium, Atorvastatin-Aliskiren, Atosiban, Atovaquone, Atrinositol, Auristatin E, Aurothioglucose, Australifungin, Australine, Avicenol A, Avicequinone A, Avicin D, Avicin G, Avorelin, Axitirome, Azacitidine, Azaromycin SC, Azithromycin, Azithromycin Copper Complex, Bactobolin, Bafilomycin A1, Bafilomycin C1, Baicalin, Balhimycin, Bambuterol, Baogongteng A, Barixibat, Barusiban, Basifungin, Becatecarin, Beciparcil, Beclometasone dipropionate, Becocalcidiol, Bedoradrine sulfate, Befloxatone, Befunolol hydrochloride, Begacestat, Belactin B, Belotecan hydrochloride, Beloxepin, Benanomicin A, Benanomicin B, Benexate cyclodextrin, Bengazole A, Bengazole B, Beraprost sodium, Bervastatin, Beta-Boswellic Acid, beta-Hydroxy beta-methylbutyrate, Betamethasone butyrate propionate, Betamethasone dipropionate, Beta-Sialosylcholesterol Sodium Salt, Betaxolol hydrochloride, Bevantolol hydrochloride, Biapenem, Bicalutamide, Bimatoprost, Bimoclomol, Bimoclomol 1-oxide, Bimosiamose, Binodenoson, Biperiden, Bipranol hydrochloride, Bisabosqual A, Bisabosqual B, Bisabosqual C, Bisabosqual D, Bisoprolol fumarate, Bitolterol mesylate, Bleomycin A2 sulfate, Bogorol A, Bohemine, Boholmycin, Bolinaquinone, Borrelidin, Bosentan, Brasilicardin A, Brasilinolide A, Brasilinolide B, Brecanavir, Breflate, Breynin A, Breynin B, Brivanib, Brivudine, Bromocriptine mesilate, Bromperidol, Brovincamine fumarate, Bryostatin 1, Bryostatin 10, Bryostatin 11, Bryostatin 12, Bryostatin 13, Bryostatin 9, Budesonide, Bungeolic acid, Buprenorphine hemiadipate, Buprenorphine hydrochloride, Buprenorphine-Val-carbamate, Buserelin acetate, Butalactin, Buteranol, Butixocort, Butofilolol, Butorphanol tartrate, Byssochlamysol, Cabazitaxel, Cabin 1, Cadralazine, Calanolide A, Calanolide B, Calbistrin A, Calbistrin B, Calbistrin C, Calbistrin D, Calcipotriol, Calcitriol, Calcium-like peptide 1, Caloporoside B, Caloporoside C, Caloporoside D, Caloporoside E, Caloporoside F, Calphostin B, Calphostin D, Calteridol calcium, Cambrescidin 800, Cambrescidin 816, Cambrescidin 830, Cambrescidin 844, Camiglibose, Campestanol ascorbyl phosphate, Canadensol, Canagliflozin, Candelalide B, Candelalide C, Cangrelor tetrasodium, Canrenoate potassium, Canventol, Capadenoson, Capecitabine, Caprazamycin A, Caprazamycin B, Caprazamycin C, Caprazamycin E, Caprazamycin F, Capridine beta, Carabersat, Carbazomadurin A, Carbazomadurin B, Carbazomycin G, Carbazomycin H, Carbovir, Caribaeolin, Caribaeoside, Carisbamate, Carmoterol hydrochloride, Carpesterol, Carquinostatin A, Carsatrin, Carteolol hydrochloride, Carteramine A, Carvastatin, Carvedilol, Caspofungin acetate, Castanospermine, Cefbuperazone sodium, Cefcanel, Cefonicid sodium, Cefoselis sulfate, Celgosivir, Celikalim, Celiprolol hydrochloride, Cephalostatin 1, Cephalostatin 2, Cephalostatin 3, Cephalostatin 4, Cephalostatin 7, Cephalostatin 8, Cephalostatin 9, Ceramidastin, Cerebroside A, Cerebroside B, Cerebroside C, Cerebroside D, Cerivastatin sodium, Ceruletide diethylamine, Cethromycin, Cetrorelix Acetate, Chackol, Chaetoatrosin A, Chafuroside, Chenodeoxycholic acid, Chetocin, Chinoin-169, Chloptosin, Chlorazicomycin, Chlorofusin, Chlorogentisylquinone, Chloroorienticin A, Chloroorienticin B, Chlortalidone, Cholerae Autoinducer-1, Choline alfoscerate, Ciclesonide, Cidofovir, Cimaterol, Cimetropium bromide, Cinatrin A, Cinatrin B, Cinatrin C1, Cinatrin C2, Cinatrin C3, Cinnabaramide A, Cinolazepam, Ciprokiren, Citicoline, Citreamicin-eta, Citropeptin, Citrullimycine A, Cladribine, Clarithromycin, Clavaric acid, Clavarinone, Clavulanate potassium, Clazosentan, Clevudine, Clidinium bromide, Clindamycin hydrochloride, Clitocine, Clobenoside, Clofarabine, Clopithepin, Cloranolol hydrochloride, Cocositol, Colabomycin A, Coleneuramide, Coleophomone B, Colestimide, Colforsin, Colforsin daproate hydrochloride, Colletoic acid, Colupulon, Conagenin, Coniferol Alcohol, Coniosetin, Conocurvone, Conophylline, Contignasterol, Contortumine hydrochloride, Contulakin G, Coproverdine, Correolide, Cortexolone 17alpha-propionate, Corynecandin, Cositecan, Costatolide, Coumamidine Gamma1, Coumamidine Gamma2, Crassicauline A, Crellastatin A, Crisnatol mesilate, Cromakalim, Crossoptine A, Crossoptine B, Curtisian D, Curvularol, Cyclamenol, Cyclandelate, Cyclipostin A, Cyclohexanediol, Cyclomarin A, Cyclooctatin, Cycloplatam, Cyclosporin A, Cyclosporin J, Cyclothialidine, Cygalovir, Cypemycin, Cystocin, Cystothiazole C, Cystothiazole D, Cystothiazole F, Cytallene, Cytarabine, Cytaramycin, Cytoblastin, Cytochalasin B, Cytochlor, Cytogenin, Cytosporic acid, Cytostatin, Cytotrienin I, Cytotrienin II, Cytotrienin III, Cytotrienin IV, Cytoxazone, DACH-Pt(II)-bis-ascorbate, Dacinostat, Dactimicin, Dactylfungin A, Dactylfungin B, Dactylocycline A, Dactylocycline B, Dactylorhin B, DADMe-Immucillin-G, DADMe-Immucillin-H, Dalbavancin, Dalfopristin mesilate, Dalvastatin, Dapagliflozin, Daphnodorin B, Dapitant, Dapropterin dihydrochloride, Darunavir, Dasantafil, Dasatinib, Daunorubicin, Davunetide, Decahydromoenomycin A, Decaplanin, Decarestrictine C, Decarestrictine D, Decatromicin A, Decatromicin B, Decitabine, Decursinol, Deferiprone, Deflazacort, Deforolimus, Degarelix acetate, Dehydelone, Dehydrodolastatin-13, Dehydroilludin M, Delafloxacin, Delaminomycin A, Delaminomycin B, Delaminomycin C, Delimotecan sodium, delta-Tocopherol glucoside, Deltibant, Demethimmunomycin, Demethomycin, Demethylallosamidin, Demethylasterriquinone B-1, Denopamine, Denufosol tetrasodium, Deoxyenterocin, Deoxylaidlomycin, Deoxymulundocandin, Deoxynojirimycin, Deoxyspergualin Hydrochloride, Deprodone propionate, Desacetyleleutherobin, Desacetylravidomycin N-oxide, Desacetylvinblastinehydrazide, Desacetylvinblastinehydrazide/folate conjugate, Desbutyl benflumetol, Desbutylhalofantrine hydrochloride, Desferri-danoxamine, Desferri-nordanoxamine, Desferri-salmycin A, Desferri-salmycin B, Desferri-salmycin C, Desferri-salmycin D, Desisobutyrylciclesonide, Deslorelin, Desmethyleleutherobin, Desmin-370, Desogestrel, Desoxyepothilone B, Desoxyepothilone F, Desoxylaulimalide, Desvenlafaxine succinate, Dexamethasone, Dexamethasone beloxil, Dexamethasone cipecilate, Dexamethasone Palmitate, Dexamethasone sodium phosphate, Dexanabinol, Dexelvucitabine, Dexylosylbenanomycin A, DHA-paclitaxel, Diadenosine tetraphosphate, Dictyostatin 1, Didemnin X, Didemnin Y, Dideoxyinosine, Dienogest, Diepoxin-sigma, Diflomotecan, Digalactosyldiacylglycerol, Digoxin, Diheteropeptin, Dihydro-alpha-ergokryptine mesylate, Dihydrocostatolide, Dihydroeponemycin, Dihydroergotamine mesylate, Dihydrogranaticin B, Dihydroheptaprenol, Dihydroisosteviol, Dilevalol, Dilevalol hydrochloride, Dilmapimod, Dimelamol, Dimethandrolone, Dimethylcurcumin, di-mPEG5-Atazanavir, Dinaphine, Dioncoquinone A, Dioncoquinone B, Dioxolane thymine nucleoside, Diperamycin, Dipivefrine hydrochloride, Dipyridamole, Dipyridamole beta-cyclodextrin complex, Diquafosol tetrasodium, Dirithromycin, Discodermide, Discodermide acetate, Disermolide, Disodium cromproxate, Disodium lettusate, Disorazol E1, Docetaxel, Docosanol, Docosyl cidofovir, Dofequidar fumarate, Dolastatin 13, Doramectin, Doranidazole, Doretinel, Doripenem, Dorrigocin A, Dorrigocin B, Doxefazepam, Doxercalciferol, Doxifluridine, Doxorubicin Hydrochloride, Doxorubicin, Morpholinyl, DoxoTam 12, Doxycycline hyclate, Dridocainide, Droxidopa, Droxinavir, Drupangtonine, DTPA-adenosylcobalamin, Duramycin, Dutomycin, Ecdysterone, Ecomustine, Ecraprost, Ecteinascidin 1560, Ecteinascidin 722, Ecteinascidin 729, Ecteinascidin 736, Ecteinascidin 757, Edotecarin, Edotreotide yttrium, Eicosyl cidofovir, Elacytarabine, Elansolid C1, Eldecalcitol, Eleutherobin, Eleutheroside B, Eliprodil, Elisapterosin B, Elocalcitol, Elomotecan hydrochloride, Eltanolone, Elvitegravir, Elvucitabine, Emakalim, Embeconazole, Embelin, Emestrin C, Emtricitabine, Enalkiren, Enfumafungin, Englerin A, Enigmol, Enkastin (D), Enkastin AD, Enkastin AE, Enkastin ID, Enkastin IE, Enkastin VD, Enkastin VE, Enocitabine, Enoloxone, Enpiperate, Enprostil, Enrasentan, Entecavir, ent-Estriol, Eperezolid, Eperezolid N-oxide, Epervudine, Epicochlioquinone A, Epidoxoform, Epirubicin hydrochloride, Epispongiadiol, Epocarbazolin A, Epocarbazolin B, Epofolate, Epolactaene, Eponemycin, Epoprostenol sodium, Epothilone A, Epothilone A N-oxide, Epothilone B N-oxide, Epothilone E, Epoxomicin, Epoxyvibsanin B, Eptaloprost, Eptastatin sodium, Eptastigmine Tartrate, Erabulenol B, Erectumin A, Eremomycin, Eremophyllene A, Ergotamine tartrate, Eribulin mesilate, Eriocalyxin B, Eritoran tetrasodium, Ersentilide, Ersentilide hydrochloride, Ertapenem sodium, Eryloside A, Eryloside F, Erythritol, Erythrodiol, Erythromycin, Erythromycin Acistrate, Erythromycin salnacedin, Erythromycin stinoprate, Esculeogenin A, Esculeoside A, Esmolol hydrochloride, Espatropate hydrate, Esperatrucin, Estetrol, Estradiol, Estradiol acetate, Estren, Estriol, Ethanolamine, Ethchlorvynol, Ethinylestradiol, Ethylthio-DADMe-immucillin-A, Ethynylcytidine, Etidronic acid disodium salt, Etiprednol dicloacetate, Etonogestrel, Etoposide, Etoposide phosphate disodium salt, Eugenodilol, Eugenosedin A, Euphodendroidin D, Evernimicin, Everolimus, Exatecan mesilate, Ezetimibe, Ezetimibe glucuronide, Faeriefungin A, Faeriefungin B, Faropenem medoxomil, Faropenem sodium, Fasobegron hydrochlorid, Fattiviracin A1, Febradinol, Febuprol, Fenoterol hydrobromide, Ferulinolol, Fesoterodine fumarate, Fexofenadine hydrochloride, Fidaxomicin, Filibuvir, Fimbrigal P, Fingolimod hydrochloride, Finrozole, Flomoxef Sodium, Flopristin, Floxuridine, Fluconazole, Fludarabine phosphate, Fludelone, Fludeoxyglucose (18F), Flumecinol, Flunisolide, Flunoprost, Fluocinonide, Fluoroindolocarbazole A, Fluoroindolocarbazole B, Fluoroindolocarbazole C, Fluoroneplanocin A, Fluostatin B, Flupentixol hydrochloride, Fluphenazine hydrochloride, Flurithromycin, Fluticasone furoate, Fluticasone propionate, Flutropium Bromide, Fluvastatin sodium, Fluvirucin B2, Foetidine 1, Foetidine 2, Fondaparinux sodium, Formamicin, Formestane, Formosyn A, Formoterol fumarate, Forodesine hydrochloride, Fosteabine sodium hydrate, Frederine, Fucoxanthin, Fudosteine, Fuladectin component A3, Fuladectin component A4, Fulvestrant, Fumagalone, Furaquinocin A, Furaquinocin B, Fusacandin A, Fusacandin B, Fuscoside B, Fusidate silver, Fusidienol, Gabusectin, Gabusectin methyl ester, Gadobutrol, Gadocoletic acid trisodium salt, Gadomelitol, Gadoterate meglumine, Gadoteridol, Galactomycin I, Galactomycin II, Galactosyllactose, Galamustine hydrochloride, Galantamine hydrobromide, Galarubicin hydrochloride, Galocitabine, Ganaxolone, Ganciclovir, Ganciclovir elaidic acid, Ganciclovir monophosphate, Ganciclovir Sodium, Ganefromycin Alpha, Ganefromycin Beta, Ganglioside GM1, Ganirelix, Ganirelix acetate, Ganoderic acid X, Garomefrine hydrochloride, Garveatin E, Garveatin F, Gemcitabine, Gemcitabine elaidate, Gemeprost, Genaconazole, Genipin, Gestrinone, Gilatide, Gimatecan, Girodazole, Glaucocalyxin A, Glemanserin, Glenvastatin, Glidobactin PF-1, Glucarolactam potassium, Glucolanomycin, Glucolipsin A, Glucolipsin B, Glucopiericidinol A1, Glucopiericidinol A2, Glucosamine sulfate, Glufosfamide, Glycopin, Glycopyrronium bromide, Glycothiohexide alpha, Glycyrrhizinic acid, Gomphostenin, Goodyeroside A, Goodyeroside B, Goralatide, Goserelin, Granaticin B, Griseusin C, Gypsetin, Halistatin 1, Halistatin 2, Halistatin 3, Halobetasol propionate, Halofantrine hydrochloride, Halofuginone hydrobromide, Halometasone, Haloperidol, Halopredone Acetate, Halovir A, Halovir B, Halovir C, Halovir D, Halovir E, Halxazone, Haperforin F, Haperforine A, Haperforine B1, Hatomamicin, Hatomarubigin C, Hatomarubigin D, Hattalin, Hayumicin A, Hayumicin B, Hayumicin C1, Hayumicin C2, Hayumicin D, Hederacolchiside E, Heliquinomycin, Helvecardin A, Helvecardin B, Heptaminol AMP Amidate, Heptelidic acid chlorohydrin, Hexadecyl cidofovir, Hexadecyloxypropyl-cidofovir, Hexafluorocalcitriol, Hidrosmin, Himastatin, Hispitolide C, Hispitolide D, Histrelin, Histrelin acetate, Homorisedronate, Hyaluronate sodium, Hydrocortisone Aceponate, Hydrostatin A, Hydroxychloroquine sulfate, Hydroxymycotrienin A, Hydroxymycotrienin B, Hydroxyphoslactomycin B, Hydroxyzine hydrochloride, Hypeptin, Hyperoside, Hypocholamide, Hypocholaride, Ibandronic acid monosodium salt monohydrate, Ibutilide fumarate, Icariin, Icatibant acetate, Idarubicin hydrochloride, Idebenone, Idremcinal, Ifenprodil, Ilatreotide, Iliparcil, Ilonidap, Iloprost, Imipenem, Immunosine, Implitapide, Incyclinide, Indacaterol, Indanaprost (S), Indinavir sulfate, Indomethacin-Simvastatin, Indynaprost, Ingenol mebutate, Inophyllum B, Inophyllum P, Inosiplex, Integracide A, Integracide B, Integracin B, Integramycin, Integrastatin A, Iobitridol, Iodixanol, Iodorubidazone (p), Iofratol, Iohexol, Iomeprol, Iopamidol, Iopentol, Iopromide, Iotriside, Iotrol, Ioversol, Ioxilan, Ipratropium bromide, Iralukast, Iralukast sodium, Irciniastatin A, Irciniastatin B, Irinotecan hydrochloride, Irofulven, Isalmadol, Isavuconazole, Isavuconazonium chloride hydrochloride, Isepamicin sulfate, Isodoxorubicin, Isoeleutherobin A, Isofagomine tartrate, Isofloxythepin, Isohomohalichondrin B, Isosorbide 5-mononitrate, Isospongiadiol, Isoxazoledehydelone, Isoxazolefludelone, Itavastatin calcium, Itrocinonide, Ixabepilone, Jadomycin B, Janthinomycin A, Janthinomycin B, Janthinomycin C, Jorumycin, Kadsuphilin C, Kahalalide F, Kaitocephalin, Kanamycin, Kanglemycin A, Kansuinin B, kappa-Conotoxin P VIIA, Karalicin, Katanosin A, Katanosin B, Khafrefungin, Kifunensine, Kigamicin A, Kigamicin B, Kigamicin C, Kigamicin D, Kigamicin E, Kigamicinone, Kijimicin, Kinsenoside, Kobifuranone B, Kobiin, Kodaistatin A, Kodaistatin B, Kodaistatin C, Kodaistatin D, Kosinostatin, Kuehneromycin A, Kurasoin B, Kynostatin-227, Kynostatin-272, Labedipinedilol A, Labedipinedilol B, Labetalol hydrochloride, Labradimil, Lactonamycin, Lactosylphenyl trolox, Ladirubicin, Lagatide, Laherradurin, Lamivudine, Landiolol, Lanreotide acetate, Lanthiopeptin, Larotaxel dihydrate, Lasinavir, Lasonolide A, Latanoprost, Latrunculin S, Lavanduquinocin, Lecirelin, Ledazerol, Leinamycin, Lemuteporphin, Lenapenem hydrochloride, Lenapenem hydrochloride hydrate, Leptocillin, Leptofuranin A, Leptofuranin B, Lersivirine, Lestaurtinib, Leuprolide acetate, Leurubicin, Leustroducsin A, Leustroducsin B, Leustroducsin C, Leustroducsin H, Levalbuterol hydrochloride, Levobetaxolol hydrochloride, Levobunolol hydrochloride, Levodopa 3-O-glucoside, Levodopa 4-O-glucoside, Levodropropizine, Levonadifloxacin arginine salt, Levonebivolol, Levonorgestrel, Lexacalcitol, L-Histidinol, Liblomycin, Licorice-saponin C2, Lificiguat, Limaprost alfadex, Linaprazan, Linderol A, Lipiarmycin B3, Lipiarmycin B4, Lipo-isocarbacyclin methyl ester Clinprost, Liquiritin apioside, Lisofylline, Lobatamide C, Lobatamide F, Lobophorin A, Lobophorin B, Lobucavir, Lodenafil, Lodenosine, Lonaprisan, Longestin, Loperamide hydrochloride, Lopinavir, Lorazepam, Lormetazepam, Lornoxicam, Losartan, Losartan potassium, Losigamone, Loteprednol etabonate, Lovastatin, Loxoribine, L-threitol ceramide, L-threo-C6-pyridinium-ceramide-bromide, Lubeluzole, Lubiprostone, Lumefantrine, Luminacin D, Lupulone, Lurtotecan, Lu-Tex bis(gluconate), Lysobactin, Mabuterol hydrochloride, Macquarimycin B, Macrocarpin B, Macrolactine M, Madecassic acid, Madecassoside, Madindoline A, Madindoline B, Manifaxine hydrochloride, Manitimus, Mannopeptimycin alpha, Mannopeptimycin beta, Mannopeptimycin delta, Mannopeptimycin epsilon, Mannopeptimycin gamma, Manoalide, Manumycin A, Manumycin B, Manumycin C, Manumycin E, Manumycin F, Manumycin G, Manzamine A, Manzamine D, Manzamine E, Manzamine F, Maribavir, Marimastat, Maslinic acid, Matteuorienate A, Matteuorienate B, Matteuorienate C, Mazindol, Mazokalim, Mefloquine hydrochloride, Megovalicin A, Megovalicin B, Megovalicin C, Megovalicin D, Megovalicin G, Megovalicin H, Meloxicam, Meluadrine, Meluadrine tartrate, Memno-peptide A, Mepenzolate bromide, Mepindolol sulfate, Mepindolol transdermal patch, Meropenem, Metaraminol, Metesind glucuronate, Methanobactin, Methoxatone, Methscopolamine bromide, Methyl bestatin, Methylnaltrexone bromide, Methylprednisolone, Methylprednisolone aceponate, Methylprednisolone suleptanate, Methyltestosterone, Methylthio-DADMe-immucillin-A, Methysergide maleate, Metildigoxin, Metipranolol, Metoprolol Fumarate, Metoprolol succinate, Metoprolol tartrate, Metrifonate, Metronidazole, Micacocidin A, Micacocidin B, Micafungin sodium, Michigazone, Microbisporicin A2, Microcolin A, Micronomicin sulfate, Midecamycin acetate, Mideplanin, Mifepristone, Miglitol, Miglustat, Milataxel, Milbemycin alpha-9, Milrinone Lactate, Minerval, Minocycline hydrochloride, Minodronate, Miporamicin, Mipragoside, Mirabegron, Mirodenafil hydrochloride, Misakinolide, Misoprostol, Mitemcinal fumarate, Mitoxantrone hydrochloride, Mizoribine, Modecainide, Modithromycin, Moenomycin A chloride bismuth salt, Mometasone furoate, Momordin Ic, Monamidocin, Monlicin A, Monogalactosyldiacylglycerol, Monohydroxyethylrutoside, Monophosphoryl lipid A, Montelukast sodium, Morphine Glucuronide, Morphine hydrochloride, Morphine sulfate, Motexafin gadolinium, Motexafin lutetium, Moxidectin, Mozenavir mesilate, Multiforisin A, Mumbaistatin, Mupirocin, Muraminomicin A, Muraminomicin B, Muraminomicin C, Muraminomicin D, Muraminomicin E1, Muraminomicin E2, Muraminomicin F, Muraminomicin G, Muraminomicin H, Muraminomicin I, Muraminomicin Z1, Muraminomicin Z2, Muraminomicin Z3, Muraminomicin Z4, Muramyl dipeptide C, Mureidomycin A, Mureidomycin B, Mureidomycin C, Mureidomycin D, Mureidomycin E, Mureidomycins, Mycalamide A, Mycaperoxide A, Mycaperoxide B, Mycestericin E, Mycolactone A, Mycolactone B, Myrciacitrin I, Myrciacitrin II, Myrciaphenone B, Myrocin C, Mytolbilinol, N4-Hexadecyl-dC-AZT, N-9-Oxadecyl-6-methyl-DGJ, N-Acetylsperamycin A1, N-Acetylsperamycin AlB, N-Acetylsperamycin A2, Nadifloxacin, Nadolol, Nafarelin acetate, Naftopidil, Nafuredin, Nafuredin-gamma, Nagstatin, Nalbuphine hydrochloride, Nalfurafine hydrochloride, Nalmefene, Naloxone hydrochloride, Naltrexone hydrochloride, Naltrindole, Namitecan, Napsamycin A, Napsamycin B, Napsamycin C, Napsamycin D, Nardeterol, Naroparcil, Navuridine, N-Cyclopentyl-tazopsine, Nebivolol, Nectrisine, Neldazosin, Nelfinavir mesilate, Nelivaptan, Nelzarabine, Nemifitide ditriflutate, Nemorubicin, Neocimicigenoside A, Neocimicigenoside B, Neolaulimalide, Neomycin B-arginine conjugate, Neomycin-acridine, Neotripterifordin, Nepadutant, Neparensinol A, Neridronic acid, Neristatin 1, Nesbuvir, Netilmicin sulfate, Netivudine, Neu5Ac2en, Ngercheumicin A, Ngercheumicin B, N-hexacosanol, Nifekalant hydrochloride, Nileprost beta-cyclodextrin clathrate, Nipradolol, Nitropravastatin, N-Nonyl-deoxygalactojirimycin, Nocathiacin I, Nocathiacin II, Nocathiacin III, Nocathiacin IV, N-Octyl-beta-valienamine, NO-hydrocortisone, Noladin ether, Noraristeromycin, Norelgestromin, Norethisterone, Normethyljiadifenin, Nortopixantrone hydrochloride, Nostocyclopeptide M1, Nothramicin, NO-Ursodeoxycholic acid, N-Retinoyl-D-glucosamine, Nubiotic 2, Nutlin-2, Obelmycin H, Oberadilol, Oberadilol Monoethyl Maleate, Obeticholic acid, Ocimumoside A, Ocimumoside B, Octacosamicin A, Octacosamicin B, Octreotide Acetate, O-Demethylchlorothricin, Odiparcil, Oenothein B, Okicenone, Oleanolic acid, Oleoyl-L-Valinol amide, Olmesartan, Olmesartan medoxomil, Olpadronic acid sodium salt, Omaciclovir, Ombrabulin, Ombrabulin hydrochloride, Onnamide A, Opiorphin, Opipramol hydrochloride, Orciprenaline sulphate, Orienticin A, Orienticin B, Orienticin C, Orienticin D, Oritavancin, Orniplabin, Ornoprostil, Ortataxel, Orthosomycin A, Orthosomycin B, Orthosomycin C, Orthosomycin D, Orthosomycin E, Orthosomycin F, Orthosomycin G, Orthosomycin H, Ospemifene, Osutidine, Ovalicin, Oxandrolone, Oxaspirol A, Oxaspirol B, Oxazepam, Oxazofurin, Oxeclosporin, Oxiracetam Oxitropium bromide, Oxolide, Oxprenolol hydrochloride, Oxybutynin chloride, Oxycodone hydrochloride, Oxymorphazole dihydrochloride, Oxymorphone hydrochloride, Oxymorphone-Val-carbamate, Oxynor, Oxyphencyclimine hydrochloride, Ozarelix, Pachastrissamine, Pachymedusa dacnicolor Tryptophyllin-1, Paciforgine, Paclitaxel, Paclitaxel ceribate, Paecilaminol, Paeciloquinone D, Pafenolol, Palau'amine, Paldimycin B, Palinavir, Palmidrol, Palosuran sulfate, Pamapimod, Pamaqueside, Pamidronate sodium Panamesine hydrochloride, Pancratistatin disodium phosphate, Pancratistatin-3,4-cyclic phosphate sodium salt, Panipenem, Pantethine, Papuamide A, Papuamide B, Papuamide C, Papuamide D, Papyracillic acid, Paraherquamide G, Parasin I, Paricalcitol, Parodilol Hemifumarate, Paromomycin, Parthenin, Parvisporin B, Patellazole A, Patellazole B, Patellazole C, Patupilone, Pauciflorine A, Pauciflorine B, Paulomycin, Paulomycin A2, Paulomycin B, Paulomycin C, Paulomycin D, Paulomycin E, Paulomycin F, PEG40000-Paclitaxel, PEG5000-Paclitaxel, PEG-conjugated camptothecin, PEG-vancomycin, Peloruside A, Penasterol, Penbutolol sulfate, Penciclovir, Penicillide, Pentostatin, Peplomycin, Pepluanin A, Peramivir, Percyquinnin, Periciazine, Perillyl alcohol, Perphenazine, Persin, Petrosaspongiolide M, Phaseolinone, Phenochalasin A, Phenochalasin B, Philinopside A, Phomactin A, Phomactin B, Phomactin E, Phomactin F, Phomactin G, Phomoidride A, Phomopsichalasin, Phorboxazole A, Phorboxazole B, Phospholine, Phosphostim, Picumeterol fumarate, Pimecrolimus, Pimilprost, Pindolol, Pinitol, Pipalamycin, Pipenzolate bromide, Pipotiazine, Pirarubicin, Pirbuterol hydrochloride, Pirmenol hydrochloride, Pironetin, Piroxicam, Pladienolide A, Pladienolide B, Pladienolide C, Pladienolide D, Pladienolide E, Plantagoside, Plaunotol, Plitidepsin, Pluraflavin A, Pluraflavin B, Pluraflavin E, Plusbacin A1, Plusbacin A2, Plusbacin A3, Plusbacin A4, Plusbacin B1, Plusbacin B2, Plusbacin B3, Plusbacin B4, Pneumocandin A0, Pneumocandin B1, Pneumocandin B0 2-phosphate, Pneumocandin D0, Podophyllotoxin, Poldine metilsulfate, Polyestradiol phosphate, Polyketomycin, Polymer bound human leukocyte elastase inhibitor, Popolohuanone E, Posaconazole, Posizolid, Potassium embelate, Pradimicin A, Pradimicin B, Pradimicin D, Pradimicin E, Pradimicin FA-1, Pradimicin FA-2, Pradimicin FL, Pradimicin FS ((+)-enantiomer), Pradimicin L, Pradimicin Q, Pradimicin S, Pradimicin T1, Pradimicin T2, Prasterone, Prednicarbate, Prednisolone, Prednisolone acetate, Prednisolone farnesylate, Prednisone, Preussin, Pristinamycin IIA, Probestin, Procaterol Hydrochloride Hemihydrate, Procyclidine hydrochloride, Prolylmeridamycin, Propafenone hydrochloride, Propeptin T, Propranolol hydrochloride, Prostanit, Prostatin, Prostratin, Prostratin succinate, Proxodolol, Pseudoephedrine hydrochloride, Pseudohypericin, Pseudomycin A′, Pseudomycin B′, Purpuromycin, Purvalanol A, Pycnanthuquinone A, Pycnanthuquinone B, Pyloricidin B, Pyripyropene A, Pyripyropene B, Pyripyropene C, Pyripyropene D, Pyrrocidine A, Pyrrocidine B, Pyrrolosporin A, Quartromicin A1, Quartromicin A2, Quartromicin A3, Quartromicin D1, Quartromicin D2, Quartromicin D3, Quetiapine fumarate, Quinidine, Quinoxapeptin C, Rafabegron, Raluridine, Rameswaralide, Ramoplanin A′1, Ramoplanin A′2, Ramoplanin A′3, Ramorelix, Ranimustine, Ranolazine, Rapamycin, Ravidomycin N-oxide, Ravuconazole, Razupenem, Reblastatin, Regadenoson, Relcovaptan, Remikiren mesilate, Remiprostol, Remogliflozin etabonate, Repandiol, Reproterol hydrochloride, Resiquimod, Resorthiomycin, Retapamulin, Retaspimycin hydrochloride, Revatropate, Reveromycin A, Rhodiocyanoside A, Rhodiocyanoside B, Rhodostreptomycin A, Rhodostreptomycin B, Ribavirin, Ribavirin eicosenate cis, Ribavirin eicosenate trans, Ribavirin elaidate, Ribavirin oleate, Rifabutin, Rifalazil, Rifamexil, Rifampicin, Rifapentine, Rifaximin, Rilmakalim hemihydrate, Rimexolone, Rimoterol hydrobromide, Risedronate sodium, Ritipenem acoxil, Ritonavir, Rivastigmine tartrate, Rivenprost, Rocagloic acid, Rocuronium bromide, Rofleponide, Rofleponide palmitate, Rohitukine, Rokitamycin, Rolliniastatin 1, Romurtide, Rosaprostol sodium, Roscovitine, Roselipin 1A, Roselipin 1B, Roselipin 2A, Roselipin 2B, Rostafuroxine, Rosuvastatin calcium, Rosuvastatin sodium, Rotigaptide, Roxatidine bismuth citrate, Roxithromycin, Rubiginone A1, Rubiginone A2, Rubiginone B1, Rubiginone C1, Rubitecan, Ruboxyl, Rugatocenone B, Rumycin 1, Rumycin 2, Sabarubicin hydrochloride, Safingol, Saishin N, Sakyomicin A, Sakyomicin E, Salbostatin, Salbutamol nitrate, Salbutamol sulfate, Salicylihalamide A, Salicylihalamide B, Salinamide A, Salinosporamide A, Saliphenylhalamide, Salmaterol, Salmeterol xinafoate, Samaderine X, Sanfetrinem, Sanfetrinem cilexetil, Sanfetrinem sodium, Sanglifehrin A, Sanglifehrin B, Sanglifehrin C, Sanglifehrin D, Sapacitabine, Saquinavir, Saquinavir mesilate, Sarcophytol A, Sarcophytol B, Saricandin, Saussureamine D, Saussureamine E, Saxagliptin, Sazetidine-A, Schizandrin, Scopinast fumarate, Scopolamine, Scyphostatin, Secalciferol, Secobatzelline A, Secobatzelline B, Secoisolariciresinol diglucoside, Securioside A, Securioside B, Selamectin, Selank, Selodenoson, Semagacestat, Semduramicin, Semorphone hydrochloride, Seocalcitol, Seprilose, Sergliflozin etabonate, Serofendic acid, Sessiloside, Setamycin, Setazindol, Shepherdin, Shishijimicin A, Shishijimicin B, Shishijimicin C, Sialosylcholesterol-Alpha Sodium Salt, Sibanomicin, Sibiskoside, Silodosin, Siltenzepine, Silychristin, Simotaxel, Simvastatin, Sitostanol ascorbyl phosphate, Siwenmycin, Sizofiran, Smilagenin, Socorromycin, Sodium cromoglycate, Sodium oxybate, Solabegron hydrochloride, Solidagenon, Solpecainol hydrochloride, Sonedenoson, Soraprazan, Sorbicillactone A, Sorivudine, so-Simvastatin-6-one, Sotalol hydrochloride, Sparoxomycin A1, Sparoxomycin A2, Sperabillin A, Sperabillin B, Sperabillin C, Sperabillin D, Sphingofungin F, Spinorphin, Spiralizone B, Spirocardin A, Spirocardin B, Spiruchostatin A, Spiruchostatin B, Spisulosine, Spongiadiol, Spongistatin 1, Spongistatin 3, Spongistatin 4, Spongistatin 5, Spongistatin 6, Spongistatin 7, Spongistatin 8, Spongistatin 9, Sporeamicin A, Sporeamicin B, Squalamine lactate, Squalestatin I, Stachybocin A, Stachybocin B, Stachybocin C, Stachybotrin C, Stachybotrydial, Staplabin, Starrhizin, Stavudine, Stelleramacrin A, Stelleramacrin B, Sterenin A, Streptomycin, Styloguanidine, Suberosenol A, Sufotidine bismuth citrate, Sugammadex sodium, Sulfinosine, Sulfircin C, Sulopenem, Sulopenem etzadroxil, Sulphoquinovosyldiacylglycerol, Sulprostone, Sulukast, Sunflower trypsin inhibitor-1, Suplatast tosilate, Suronacrine maleate, Swiftiapregnene, Synadenol, Synguanol, Syriacusin B, Syzygiol, Tacalcitol, Tacapenem pivoxil, Taccalonolide E, Tacrolimus, Tafluprost, Takanawaene A, Takanawaene B, Takanawaene C, Talibegron, Talibegron hydrochloride, Tamandarin A, Tamandarin B, Tamolarizine Hydrochloride, Tanespimycin, TAP-doxorubicin, Taurohyodeoxycholic acid, Tautomycin, Taxuspain D, Taxuyunnanine, Tazopsine, Tebipenem, Tebipenem cilexetyl, Tebipenem pivoxil, Tecadenoson, Teicoplanin-A2-1, Teicoplanin-A2-2, Teicoplanin-A2-3, Teicoplanin-A2-5, Telavancin hydrochloride, Telbivudine, Telinavir, Telithromycin, Temazepam, Temiverine, Temiverine hydrochloride hydrate, Tempol, Temsirolimus, Temurtide, Tenidap, Teniposide, Tenoxicam, Tenuifoliside A, Tenuifoliside B, Tenuifoliside C, Tenuifoliside D, Terbutaline sulfate, Terestigmine tartrate, Terfenadine, Teriflunomide, Terlakiren, Ternatin, Terreulactone A, Terreulactone B, Terreulactone C, Terreulactone D, Tertatolol hydrochloride, Tesetaxel, Testosterone glucoside, Tetracosyl cidofovir, Tetracycline hydrochloride, Tetrafibricin, Tetrahydrocortisol, Tetrahydroechinocandin B, Tetrahydroswertianolin, Tetrahydroxyquinone, Tetromycin A, Tetromycin B, Tetronothiodin, Texenomycin A, Tezacitabine, Tezosentan, Tezosentan disodium, Thenorphine, Theopederin D, Theoperidin E, Theophylline rutoside, Thermozymocidin, Thiamet-G, Thiamphenicol, Thiarubrine E, Thiarubrine F, Thiarubrine G, Thiarubrine H, Thiazinotrienomycin B, Thiazohalostatin, Thielocin, Thiofedrine, Thiomarinol, Thiomarinol B, Thiomarinol C, Thiomarinol D, Thiomarinol E, Thiomarinol F, Thioviridamide, Thioxamycin, Thrazarine, Thymallene, Thymectacin, Tibolone, Tidembersat, Tienoxolol hydrochloride, Tigecycline, Tilisolol hydrochloride, Timolol hemihydrate, Timolol maleate, Tiotropium bromide, Tipranavir, Tiqueside, Tisocalcitate, Tixocortol buryrate propionate, Toborinone, Tobramycin, Toloxatone, Tolvaptan, Tolytoxin, Tomatine, Tomeglovir, Tonabersat, Topixantrone hydrochloride, Topotecan Acetate, Topotecane Hydrochloride, Torcitabine, Torezolid, Toripristone, Tosagestin, Tosedostat, Trabectedin, Tradecamide, Tramadol hydrochloride, Tramadol N-oxide, Trantinterol hydrochloride, Travoprost, Traxoprodil, Traxoprodil mesylate, Trecadrine, Trecetilide fumarate, Treprostinil diethanolamine, Treprostinil sodium, Trewiasine, Triamcinolone acetonide, Triamcinolone hexacetonide, Trichodimerol, Trichomycin A, Trichostatin D, Triciferol, Triciribine, Triciribine phosphate, Trifluridine, Trihexyphenidyl hydrochloride, Trilostane, Trimazosin hydrochloride, Trimegestone, Trimoprostil, Tripterifordin, Tripterin, Tripterinin, Triptolide, Troxacitabine, Tsukubamycin A, Tubelactomicin A, Tuberactomycin B, Tuberactomycin D, Tuberactomycin E, Tubingensin B, Tuftsin, Tulathromycin A, Tulathromycin B, Tulobuterol hydrochloride, Turbostatin 1, Turbostatin 2, Turbostatin 3, Turbostatin 4, Tyroservatide, Ubenimex, Ukrain, Uncarinic acid A, Uncarinic acid B, Uncialamycin, Unoprostone, Unoprostone isopropyl ester, Ursodeoxycholic acid, Ustilipid A, Ustilipid B, Ustilipid C, Uvalol, Valganciclovir hydrochloride, Valnemulin, Valonomycin A, Valopicitabine, Valrubicin, Vancomycin hydrochloride, Vancoresmycin, Vanidipinedilol, Vaninolol, Variapeptin, Veinamitol, Velnacrine Maleate, Velusetrag, Venlafaxine hydrochloride, Venlafaxine N-oxide, Vermisporin, Vernakalant hydrochloride, Verticillatine, Vicenistatin, Vildagliptin, Vincristine Sulfate, Vindesine, Vinflunine, Vinfosiltine sulfate, Vinleucinol, Vinorelbine, Vinylamycin, Viquidacin, Viramidine Hydrochloride, Viranamycin-A, Viranamycin-B, Viscosin, Vitilevuamide, Voclosporin, Voglibose, Volinanserin, Volpristin, Voriconazole, Woodorien, Xamoterol Fumarate, Xanthofulvin, Xenovulene A, Xylocydine, Yohimbine, Zahavin B, Zalcitabine, Zampanolide, Zanamivir, Zankiren, Zanoterone, Zaragozic acid D3, Z-Eleutherobin, Zidovudine, Zilascorb (2H), Zilpaterol, Zoledronic acid monohydrate, Zorubicin hydrochloride, Zosuquidar trihydrochloride, Zotarolimus, Zoticasone propionate, Zuclopenthixol hydrochloride.

Suitable drugs containing aromatic hydroxyl groups are, for example, (−)-cis-Resorcylide, (−)-Indocarbazostatin B, (−)-Salmeterol, (−)-Subersic acid, (+)-alpha-Viniferin, (+)-Etorphine, (+)-Indocarbazostatin, (+)-SCH-351448, (R)-Gossypol, (S)-(+)-Curcuphenol, (S)-Methylnaltrexone bromide, [8]-Gingerol, [Arg(Me)9] MS-10, [D-Tyr1,Arg(Me)9] MS-10, [D-Tyr1,AzaGly7,Arg(Me)9] MS-10, [D-Tyr1] MS-10, [psi[CH2NH]Tpg4]Vancomycin aglycon, [Trp19] MS-10, 13-Deoxyadriamycin hydrochloride, 14-Methoxymetopon, 14-Phenylpropoxymetopon, 18,19-Dehydrobuprenorphine hydrochloride, 2,12-Dimethyleurotinone, 2′-Hydroxymatteucinol, 2-Methoxyestradiol, 2-Methyleurotinone, 3,5-Dicaffeoylquinic acid, 3-Bromodiosmetine, 3-Bromodiosmine, 3-Chlorodiosmetine, 3-Chlorodiosmine, 4′,7,8-Trihydroxyisoflavone, 4-Aminosalicylic acid, 4-Hydroxyatomoxetine, 4-Iodopropofol, 5-Iodofredericamycin A, 5Z-7-Oxozeaenol, 6-Carboxygenistein, 6-O-mPEG4-Nalbupine, 6-O-mPEG5-Nalbuphine, 7-Methylcapillarisin, 8(R)-Fluoroidarubicin hydrochloride, 8′,9′-Dehydroascochlorin, 8-Carboxy-iso-iantheran A, 8-Paradol, 8-Prenylapigenin, 8-Prenylnaringenin, 9-Hydroxycrisamicin A, A-42867 pseudoaglycone, Abarelix, Acacetin, Aclarubicin, Acolbifene hydrochloride, Acotiamide hydrochloride hydrate, Acrovestone, Actinoplanone A, Actinoplanone B, Aculeacin Agamma, Adaphostin, Adarotene, Adxanthromycin A, Aerothricin 1, Aerothricin 16, Aerothricin 41, Aerothricin 45, Aerothricin 50, Aerothricin 55, Ajulemic acid, Alchemix, Aldifen, alpha-Mangostin, alpha-Methylepinephrine, alpha-Methylnorepinephrine, Alpha-Peltatin, Altromycin A, Altromycin B, Altromycin C, Altromycin D, Altromycins, Alvimopan hydrate, Alvocidib hydrochloride, Amamistatin A, Amamistatin B, Amarogentin, Amelubant, Amidox, Aminocandin, Amodiaquine, Amoxicillin trihydrate, Amrubicin Hydrochloride, Amurensin H, Anguillosporal, Anidulafungin, Ankinomycin, Annamycin, Annulin C, Antimycin A11, Antimycin A12, Antimycin A13, Antimycin A14, Antimycin A15, Antimycin A16, Apicularen A, Apicularen B, Apigenin, Apomine, Apomorphine hydrochloride, Arbidol, Arbutamine hydrochloride, Arformoterol tartrate, Artepillin C, Arzoxifene hydrochloride, Aspoxicillin, Atalaphillidine, Atalaphillinine, Atraric acid, Avorelin, Axitirome, Azaresveratrol, Azatoxin, Azepinostatin, Baicalein, Baicalin, Balhimycin, Balsalazide disodium, Banoxantrone, Bazedoxifene acetate, Bazedoxifene hydrochloride, Bedoradrine sulfate, Benadrostin, Benanomicin A, Benanomicin B, Benastatin A, Benastatin B, Benastatin C, Benastatin D, Benzbromarone, Berefrine, Berupipam maleate, beta-Mangostin, Biemnidin, Biochanin A, Bioxalomycin alpha 1, Bioxalomycin alpha2, Bismuth subsalicylate, Bisphenol, Bix, Bizelesin, Bogorol A, Brandisianin A, Brandisianin B, Brandisianin C, Brasilicardin A, Brevifolin carboxylic acid, Breynin A, Breynin B, Bromotopsentin, Buflomedil pyridoxalphosphate, Buprenorphine hydrochloride, Buserelin acetate, Butein, Buteranol, Butorphan, Butorphanol tartrate, Calebin A, Calocoumarin A, Caloporoside D, Caloporoside E, Caloporoside F, Calphostin A, Calphostin B, Calphostin C, Calphostin D, Calphostin I, Capillarisin, Capsazepine, Carbazomadurin A, Carbazomadurin B, Carbetocin, Carbidopa, Carmoterol hydrochloride, Caspofungin acetate, Cassigalol A, Cefetecol, Cefoperazone sodium, Cefpiramide sodium, Cefprozil, Cefprozil monohydrate, Cetrorelix Acetate, Chaetoatrosin A, Chafuroside, Chloroorienticin A, Chloroorienticin B, Chondramide A, Chondramide B, Chondramide C, Cinnatriacetin A, Cinnatriacetin B, cis-6-Shogaol, Citpressine I, Citreamicin-Alpha, Citreamicin-eta, Citrusinine-I, Clausenamine A, Combretastatin A-1, Combretastatin A-2, Combretastatin A-3, Combretastatin B-1, Combretastatin B-2, Combretastatin B-3, Combretastatin B-4, Combretastatin D-1, Combretastatin D-2, Complestatin, Coniferol Alcohol, Conophylline, Corynecandin, Cosalane, Crisamicin C, Crobenetine, Crobenetine hydrochloride, Curtisian A, Curtisian B, Curtisian D, Cyanidin Chloride Monohydrate, Cyclocommunol, Cycloproparadicicol, Cyclotheonamide A, Cyclothialidine, Cyrtominetin, Cytogenin, Cytosporone B, Cytotrienin I, Cytotrienin II, Dactylocycline A, Dactylocycline B, Dalargin, Dalbavancin, Damunacantal, Daphnodorin A, Daphnodorin B, Daphnodorin C ((−)-enantiomer), Darbufelone, Darbufelone mesilate, Daunorubicin, Daurichromenic acid, Davidigenin, Deacetyl moxisylyte hydrochloride, Decaplanin, Decyl gallate, Deferasirox, Dehydrozingerone, Delphinidin, Denopamine, Deoxymulundocandin, Dersalazine, Desacetylravidomycin N-oxide, Desglugastrin tromethamine, Deslorelin, Desmopressin acetate, Desvenlafaxine succinate, Dexanabinol, Dextrorphan, Dexylosylbenanomycin A, D-Fluviabactin, Diazaphilonic acid, Diazepinomicin, Dieckol, Diflunisal, Dihydrexidine, Dihydroavenanthramide D, Dihydrogranaticin B, Dihydrohonokiol B, Dihydroraloxifene, Dilevalol, Dilevalol hydrochloride, Dinapsoline, Dinoxyline, Dioncoquinone A, Dioncoquinone B, Dipotassium gossypolate, Dobutamine hydrochloride, Dobutamine Phosphate, Dopexamine, Dopexamine hydrochloride, Dosmalfate, Doxorubicin Hydrochloride, Doxorubicin, Morpholinyl, DoxoTam 12, Doxycycline hyclate, Dronabinol, Droxidopa, Duocarmycin B1, Duocarmycin B2, Duocarmycin C1, Duocarmycin C2, Dutomycin, Dynemicin A, Dynemicin C, Econazole Sulfosalicylate, Ecopipam, Ecteinascidin 1560, Ecteinascidin 722, Ecteinascidin 729, Ecteinascidin 736, Ecteinascidin 745, Ecteinascidin 757, Ecteinascidin 770, Ecteinascidin 875, Edotecarin, Edotreotide yttrium, Eflucimibe, Eflumast, Elansolid C1, Eldacimibe, Ellagic acid-4-gallate, Elliptinium acetate, Elsibucol, Eltrombopag olamine, Emodin, Enazadrem, Enofelast, Entacapone, ent-Estriol, Epidoxoform, Epigallocatechin-3-gallate, Epirubicin hydrochloride, Eplivanserin, Eplivanserin fumarate, Eplivanserin mesilate, Epocarbazolin A, Epocarbazolin B, Eprotirome, Eptazocine hydrobromide, Erabulenol A, Erabulenol B, Eremomycin, Estetrol, Estradiol, Estriol, Etalocib sodium, Etamsylate, Ethinylestradiol, Ethyl gallate, Etoposide, Eurotinone, Euxanthone, Evernimicin, Exifone, Ezetimibe, Fadolmidine hydrochloride, Feglymycin, Fenoldopam mesilate, Fenoterol hydrobromide, Fidaxomicin, Fidexaban, Fluostatin A, Fluostatin B, Foetidine 1, Foetidine 2, Folipastatin, Formobactin, Formoterol fumarate, Fosopamine, Frederine, Fulvestrant, Furaquinocin A, Furaquinocin B, Fusacandin A, Fusacandin B, Fusidienol, Galactomycin I, Galactomycin II, Galarubicin hydrochloride, Galocitabine, Gambogic acid, gamma-Mangostin, gamma-Tocotrienol, Ganirelix, Ganirelix acetate, Garvalone C, Garveatin E, Garveatin F, Genistein-7-phosphate, Gigantol, Gilvusmycin, Glucopiericidinol A1, Glucopiericidinol A2, Gludopa, Glycothiohexide alpha, Goserelin, Granaticin B, Griseusin C, Hatomarubigin A, Hatomarubigin B, Hatomarubigin C, Hatomarubigin D, Hayumicin A, Hayumicin B, Hayumicin C1, Hayumicin C2, Hayumicin D, Heliquinomycin, Helvecardin A, Helvecardin B, Hericenal A, Hericenal B, Hericenal C, Hidrosmin, Histrelin, Histrelin acetate, Hongoquercin A, Hongoquercin B, Honokiol diepoxide, Honokiol diepoxide, Human angiotensin II, Hydromorphone methiodide, Hymenistatin 1, Hypeptin, Hypericin, Hyperoside, Icariin, Idarubicin hydrochloride, Idronoxil, Ifenprodil, Imidazoacridinone, Incyclinide, Indacaterol, Indanocine, Integracin A, Integracin B, Integracin C, Integramycin, Integrastatin A, Integrastatin B, Intoplicine, Iodochlorhydroxyquin, Iododiflunisal, Iodorubidazone (p), Iolopride (123I), Ioxipride, Iralukast, Iralukast sodium, Irciniastatin A, Irciniastatin B, Isalmadol, Isobavachalcone, Isodoxorubicin, Iso-iantheran A, Isoliquiritigenin, Isomolpan Hydrochloride, Isoquine, Isovanihuperzine A, Jadomycin B, Jasplakinolide, Kadsuphilin C, Kaitocephalin, Kampanol A, Kampanol B, Kanglemycin A, Kapurimycin A1, Kapurimycin A3, Kapurimycin A3, Kehokorin D, Kehokorin E, Kigamicin A, Kigamicin B, Kigamicin C, Kigamicin D, Kigamicin E, Kigamicinone, Kistamicin A, Klainetin A, Klainetin B, Kodaistatin A, Kodaistatin B, Kodaistatin C, Kodaistatin D, Korupensamine A, Korupensamine B, Korupensamine C, Korupensamine D, Kosinostatin, Labetalol hydrochloride, Laccaridione A, Lactonamycin, Lactosylphenyl trolox, Ladirubicin, Lamellarin alpha 20-sulfate sodium salt, Lamifiban, Lanreotide acetate, Lasofoxifene, Lasofoxifene tartrate, Latamoxef sodium, L-Chicoric acid, L-Dopamide, Lecirelin, Ledazerol, Leuprolide acetate, Leurubicin, Levalbuterol hydrochloride, Levodopa, Levodopa 3-O-glucoside, Levodopa 4-O-glucoside, Levorphanol tartrate, L-Fluviabactin, Lipiarmycin B3, Lipiarmycin B4, Liquiritin apioside, Lithospermic acid B magnesium salt, Lobatamide C, Lobatamide F, Loloatin B, Luminacin D, Luteolin, Macrocarpin A, Macrocarpin B, Makaluvamine D, Makaluvamine E, Malonoben, Maltolyl p-coumarate, Mannopeptimycin beta, Manzamine F, Marinopyrrole A, Marmelin, Masoprocol, Mastprom, Matteuorienate A, Matteuorienate B, Matteuorienate C, Medicarpin, Melevodopa hydrochloride, Mellein, Meluadrine, Meluadrine tartrate, Memno-peptide A, Meptazinol hydrochloride, Mesalazine, Metaraminol, Methanobactin, Methyl gallate, Methyldopa, Methylnaltrexone bromide, Metirosine, Micacocidin A, Micacocidin B, Micafungin sodium, Michellamine B, Mideplanin, Mimopezil, Minocycline hydrochloride, Miproxifene, Mitoxantrone hydrochloride, Mivazerol, Modecainide, Mollugin, Monohydroxyethylrutoside, Morphine Glucuronide, Morphine hydrochloride, Morphine sulfate, Moxifetin hydrogen maleate, Mumbaistatin, Mureidomycin A, Mureidomycin B, Mureidomycin C, Mureidomycin D, Mureidomycin E, Mureidomycin F, Mureidomycins, Mycophenolate Mofetil, Mycophenolic acid sodium salt, Myrciacitrin I, Myrciacitrin II, Myrciaphenone B, Myriceric acid A, Mytolbilin, Mytolbilin acid, Mytolbilin acid methyl ester, Mytolbilinol, Naamidine A, Nabilone, N-Acetylcolchinol, Nafarelin acetate, Nalbuphine hydrochloride, Nalfurafine hydrochloride, N-Allylsecoboldine, Nalmefene, Naloxone hydrochloride, Naltrexone hydrochloride, Naltrindole, Napsamycin A, Napsamycin B, Napsamycin C, Napsamycin D, Nardeterol, N-Cyclopentyl-tazopsine, Nebicapone, Nelfinavir mesilate, Nemorubicin, Neparensinol A, Neparensinol B, Neparensinol C, Nerfilin I, Nicanartine, Nitecapone, Nocardione A, Nocathiacin I, Nocathiacin III, Nocathiacin IV, NO-Mesalamine, Nordamunacantal, Nostocyclopeptide M1, Nothramicin, N-tert butyl isoquine, Obelmycin H, Ochromycinone, Octyl gallate, Odapipam acetate, O-Demethylchlorothricin, O-Demethylmurrayafoline A, Oenothein B, Okicenone, Olanzapine pamoate, Olcegepant, Olsalazine sodium, Onjixanthone I, Onjixanthone II, Oolonghomobisflavan A, Oolonghomobisflavan C, Orciprenaline sulphate, Orienticin A, Orienticin B, Orienticin C, Orienticin D, Oritavancin, Orniplabin, Orthosomycin A, Orthosomycin B, Orthosomycin C, Orthosomycin D, Orthosomycin E, Orthosomycin F, Orthosomycin G, Orthosomycin H, Osutidine, Oximidine III, Oxymetazoline hydrochloride, Oxymorphazole dihydrochloride, Oxymorphone hydrochloride, Oxyphenarsine, Ozarelix, Paeciloquinine A, Paeciloquinine D, Paeciloquinone B, Paeciloquinone D, Pancratistatin-3,4-cyclic phosphate sodium salt, Pannorin, Papuamide A, Papuamide B, Papuamide C, Papuamide D, Paracetamol, Parvisporin B, PEG-vancomycin, Penicillide, Pentazocine hydrochloride, Pepticinnamin E, Phaffiaol, Phakellistatin 7, Phakellistatin 8, Phakellistatin 9, Phenochalasin A, Phentolamine mesilate, Phlorofucofuroeckol, Phomopsichalasin, Phthalascidin, Physostigmine salicylate, Piceatannol, Pidobenzone, Pinocembrin, Pipendoxifene, Pirarubicin, Pittsburgh Compound B, Platencin, Platensimycin, Pluraflavin A, Pluraflavin B, Pluraflavin E, Pneumocandin A0, Pneumocandin B0, Pneumocandin B0 2-phosphate, Pneumocandin D0, Polyestradiol phosphate, Polyketomycin, Popolohuanone E, Pradimicin A, Pradimicin B, Pradimicin D, Pradimicin E, Pradimicin FA-1, Pradimicin FA-2, Pradimicin FL, Pradimicin FS ((+)-enantiomer), Pradimicin L, Pradimicin Q, Pradimicin S, Pradimicin T1, Pradimicin T2, Prinaberel, Probucol, Procaterol Hydrochloride Hemihydrate, Propofol, Propyl gallate, Protocatechuic acid, Protocatechuic aldehyde, Pseudohypericin, Purpuromycin, Pyrindamycin A, Pyrindamycin B, Quercetin-3-O-methyl ether, Quinagolide hydrochloride, Quinobene, rac-Apogossypolone, Rac-Tolterodine, Raloxifene hydrochloride, Ramoplanin A′1, Ramoplanin A′2, Ramoplanin A′3, Ramorelix, Ravidomycin N-oxide, Rawsonol, Reblastatin, Reproterol hydrochloride, Resobene, Resorthiomycin, Retaspimycin hydrochloride, Rhodiocyanoside B, Rhododaurichromanic acid A, Rifabutin, Rifalazil, Rifamexil, Rifampicin, Rifapentine, Rifaximin, Rimoterol hydrobromide, Riodoxol, Rohitukine, Rotigaptide, Rotigotine, Roxindole Mesilate, Ruboxyl, Rufigallol, Rumycin 1, Rumycin 2, Russuphelin A, Sabarubicin hydrochloride, Saintopin, Saintopin E, Sakyomicin A, Sakyomicin E, Salazopyridazin, Salbutamol nitrate, Salbutamol sulfate, Salcaprozic acid sodium salt, Salicylazobenzoic acid, Salicylihalamide A, Salicylihalamide B, Saliphenylhalamide, Salmaterol, Salmeterol xinafoate, Saloxin, Salvianolic acid L, Sampatrilat, Sanglifehrin A, Sanglifehrin B, Sanglifehrin C, Sanglifehrin D, Saptomycin D, Sapurimycin, Saricandin, Secoisolariciresinol diglucoside, Seglitide, Semorphone hydrochloride, Shishijimicin A, Shishijimicin B, Shishijimicin C, Sibenadet hydrochloride, Silychristin, Sinomenine, Sivifene, Siwenmycin, Sootepenseone, Spinorphin, Spinosulfate A, Spinosulfate B, Spiroximicin, Stachybocin A, Stachybocin B, Stachybocin C, Stachybotrin C, Stachybotrydial, Staplabin, Sterenin A, Sterenin C, Sterenin D, Streptopyrrole, Succinobucol, Sulfasalazine, Sulphazocine, Susalimod, Symbioimine, Syriacusin A, Syriacusin B, Syriacusin C, Tageflar, Taiwanhomoflavone A, TAP-doxorubicin, Tapentadol hydrochloride, Taramanon A, Tazofelone, Tazopsine, Tebufelone, Technetium Tc 99m depreotide, Teicoplanin-A2-1, Teicoplanin-A2-2, Teicoplanin-A2-3, Teicoplanin-A2-3, Teicoplanin-A2-5, Telavancin hydrochloride, Temoporfin, Teniposide, Tenuifoliside A, Tenuifoliside B, Tenuifoliside C, Terbutaline sulfate, Terprenin, Tetracycline hydrochloride, Tetragalloylquinic acid, Tetrahydrocurcumin, Tetrahydroechinocandin B, Tetrahydroswertianolin, Thenorphine, Theophylline rutoside, Thiazinotrienomycin B, Thiazinotrienomycin F, Thiazinotrienomycin G, Thielavin G, Thielocin B3, Thymopentin, Tigecycline, Tipelukast, Tocotrienol, Tokaramide A, Tolcapone, Tolterodine Tartrate, Topotecan Acetate, Topotecane Hydrochloride, Topsentine B1, Trabectedin, trans-Resveratrol, Traxoprodil, Traxoprodil mesylate, Trimidox, Triphendiol, Troglitazone, Tubastrine, Tubulysin A, Tubulysin B, Tubulysin C, Tucaresol, Tyropeptin A10, Tyropeptin A6, Tyropeptin A9, Tyroservatide, Tyrphostin 47, Uncarinic acid A, Uncarinic acid B, Uncialamycin, Valrubicin, Vancomycin hydrochloride, Veinamitol, Venorphin, Verticillatine, Vexibinol, Vialinin B, Vinaxanthone, W Peptide, Wiedendiol A, Wiedendiol B, Woodorien, Xamoterol Fumarate, Xanthoangelol E, Xanthofulvin, Xanthomegnin, Xipamide, Yatakemycin, Zelandopam hydrochloride, Zorubicin hydrochloride.

Suitable drugs with a carboxyl group may be be selected from the list containing (−)-Subersic acid, (+)-Deoxoartelinic acid, (+)-Hemipalmitoylcarnitinium, (+)-Indobufen, (+)-SCH-351448, (E)-p-Coumaroylquinic acid, (Z)-Indenaprost, [111In-DTPA-Prol,Tyr4]bombesin, [90Y]-DOTAGA-substance P, [psi[CH2NH]Tpg4]Vancomycin aglycon, 111In-Pentetreotide, 11-Keto-Beta-Boswellic Acid, 15-Methoxypinusolidic acid, 1-Methyl-D-tryptophan, 3,5-Dicaffeoylquinic acid, 3-MATIDA, 3-O-Acetyloleanolic acid, 4-Aminosalicylic acid, 6alpha-Fluoroursodeoxycholic acid, 6-Carboxygenistein, 7-Chlorokynurenic acid, 8-Carboxy-iso-iantheran A, 99mTc-c(RGDfK*)2HYNIC, A-42867 pseudoaglycone, Aceclofenac, Acemetacin, Aceneuramic acid sodium salt, Acetyl-11-Keto-Beta-Boswellic Acid, Acetyl-Beta-Boswellic Acid, Acetylcysteine, Achimillic Acids, Acipimox, Acitazanolast, Acrivastine, Actarit, Adapalene, Adarotene, Ademetionine tosylate sulfate, Adxanthromycin A, Ajulemic acid, Alacepril, Aladapcin, Aleglitazar, Alitretinoin, Alminoprofen, Alogliptin benzoate, alpha-Linolenic acid, alpha-Lipoic acid, alpha-Methyltryptophan, Alprostadil, Altemicidin, Alutacenoic acid B, Alvimopan hydrate, Amiglumide, Amineptine, Aminocaproic acid, Aminolevulinic acid hydrochloride, Amlexanox, Amoxicillin trihydrate, Amphotericin B, Amsilarotene, Anakinra, Antiflammin-1, Antiflammin-2, Antiflammin-3, Apalcillin sodium, Aplaviroc hydrochloride, Argatroban monohydrate, Argimesna, Artelinate, Artepillin C, Artesunate, Arundifungin, Ascosteroside, Asiatic acid, Aspirin, Aspoxicillin, Assamicin I, Assamicin II, Ataluren, Atorvastatin, Atorvastatin calcium, Atrasentan, Azaromycin SC, Azelaic Acid, Azepinostatin, Azilsartan, Azoxybacilin, Aztreonam, Aztreonam L-lysine, Azumamide E, Baclofen, Bafilomycin C1, Baicalin, Balhimycin, Balofloxacin, Balofloxacin dihydrate, Balsalazide disodium, Bamirastine hydrate, Belactosin A, Belactosin C, Benanomicin A, Benanomicin B, Benastatin A, Benastatin B, Benazepril hydrochloride, Benthocyanin A, Bepotastine besilate, Beraprost sodium, Besifloxacin hydrochloride, Beta-Boswellic Acid, beta-Hydroxy beta-methylbutyrate, Betamipron, Beta-Sialosylcholesterol Sodium Salt, Bevirimat, Bexarotene, Bezafibrate, Biapenem, Bilastine, Bimosiamose, Bindarit, Binfloxacin, Biphenyl-indanone A, Boc-Belactosin A, Borrelidin, Brasilicardin A, Brasilinolide A, Bremelanotide, Brevifolin carboxylic acid, Bucillamine, Bumetanide, Bungeolic acid, Buprenorphine hemiadipate, Buprenorphine-Val-carbamate, Butibufen, Butoctamide hemisuccinate, Butyzamide, Cabin 1, Cadrofloxacin hydrochloride, Calbistrin A, Calbistrin B, Calbistrin C, Calbistrin D, Calcium-like peptide 1, Calcium-like peptide 2, Caloporoside B, Caloporoside C, Caloporoside D, Caloporoside E, Caloporoside F, Calpinactam, Calteridol calcium, Camprofen, Candesartan, Candoxatril, Candoxatrilat, Canfosfamide hydrochloride, Canrenoate potassium, Caprazamycin A, Caprazamycin B, Caprazamycin C, Caprazamycin E, Caprazamycin F, Captopril, Carbidopa, Carmoxirole hydrochloride, Carprofen, Cefaclor, Cefalexin monohydrate, Cefbuperazone sodium, Cefcanel, Cefdaloxime, Cefdinir, Cefetecol, Cefixime, Cefmatilen hydrochloride hydrate, Cefmenoxime hydrochloride, Cefminox sodium, Cefodizime, Cefonicid sodium, Cefoperazone sodium, Cefoselis sulfate, Cefotiam hydrochloride, Cefoxitin, Cefpimizole sodium, Cefpiramide sodium, Cefprozil, Cefprozil monohydrate, Ceftaroline fosamil acetate, Ceftazidime, Ceftibuten, Ceftobiprole, Cefuroxime, Ceranapril, Cerivastatin sodium, Ceruletide diethylamine, Cetefloxacin, Cetirizine hydrochloride, Chenodeoxycholic acid, Chinoin-169, Chlorambucil, Chloroorienticin A, Chloroorienticin B, Choline fenofibrate, Choline thioctate, Chrolactomycin, Cilastatin sodium, Cilazapril, Cilengitide, Cilomilast, Ciluprevir, Cinaciguat, Cinalukast, Cinatrin A, Cinatrin B, Cinatrin C1, Cinatrin C2, Cinatrin C3, Cinnatriacetin A, Cinnatriacetin B, Ciprofibrate, Ciprofloxacin hydrochloride, Circinamide, Cispentacin, Citrullimycine A, Clavaric acid, Clavulanate potassium, Clinofibrate, Clopidogrel Sulfate, Colletoic acid, Complestatin, Conagenin, Cosalane, Creatine phosphate, Cyclocreatine, Cycloplatam, Cyclothialidine, Cytomodulin, Cytosporic acid, Dabigatran, Daglutril, Dalargin, Dalbavancin, Danegaptide hydrochloride, Danofloxacin, Darinaparsin, Darusentan, Daurichromenic acid, Davunetide, Decahydromoenomycin A, Decaplanin, Decatromicin A, Decatromicin B, Deferasirox, Delafloxacin, Delapril Hydrochloride, Deltibant, Deoxylaidlomycin, Deoxynegamycin, Dersalazine, Desacetylvinblastinehydrazide/folate conjugate, Desferri-danoxamine, Desferri-nordanoxamine, Desglugastrin tromethamine, Desmin-370, Dexibuprofen, Dexibuprofen lysine, Dexketoprofen, Dexketoprofen choline, Dexketoprofen D,L-lysine, Dexketoprofen lysine, Dexketoprofen meglumine, Dexketoprofen trometamol, Dexloxiglumide, Dexpemedolac, dextro-Ciprofibrate, Dexylosylbenanomycin A, Diacerein, Diazaphilonic acid, Di-Calciphor, Difenoxin, Diflunisal, Dihydroavenanthramide D, Dihydrogranaticin B, Dihydroisosteviol, Dihydrolipoic acid, Disalazine, Disila-bexarotene, Disodium cromproxate, Disodium lettusate, Doqualast, Doripenem, Dormitroban, Dorrigocin A, Dorrigocin B, Droxidopa, DTPA-adenosylcobalamin, Duramycin, Dynemicin A, Ecabet Sodium, Ecenofloxacin hydrochloride, Econazole Sulfosalicylate, Edetic acid, Edotreotide yttrium, Efletirizine, Eflornithine hydrochloride, Eglumetad hydrate, Elansolid C1, Elarofiban, Elastatinal B, Elastatinal C, Elsibucol, Eltrombopag olamine, Elvitegravir, Emricasan, Enalapril maleate, Enalapril nitrate, Enalaprilat, Enfumafungin, Enkastin (D), Enkastin AD, Enkastin AE, Enkastin ID, Enkastin IE, Enkastin VD, Enkastin VE, Enoloxone, Enoxacin, Enrasentan, Enrofloxacin, Epalrestat, Epidioxymanadic acid A, Epidioxymanadic acid B, Epithalon, Epofolate, Epoprostenol sodium, Epostatin, Epristeride, Eprosartan mesilate, Eprotirome, Eptaloprost, Eptastatin sodium, Eptastigmine Tartrate, Eptifibatide, Erdosteine, Eremomycin, Ertapenem sodium, Ertiprotafib, Eryloside F, Esafloxacin Hydrochloride, Esonarimod, Etacrynic acid, Etalocib sodium, Etodolac, Etretin, Evatanepag, Evernimicin, Exisulind, Ezetimibe glucuronide, Fandofloxacin hydrochloride, Faranoxi, Farglitazar, Faropenem sodium, Fasobegron hydrochloride, Febuxostat, Feglymycin, Felbinac, Felbinac Lysine Salt, Fenbufen, Fexofenadine hydrochloride, Fidexaban, Finafloxacin hydrochloride, Fleroxacin, Flobufen, Flomoxef Sodium, Flunoprost, Flunoxaprofen, Flurbiprofen, Fluvastatin sodium, Folinic acid, Fondaparinux sodium, Fosfosal, Fradafiban, Frusemide, Fudosteine, Furprofen, G1 peptide, Gabadur, Gabapentin, Gabapentin enacarbil, Gabusectin, Gadobenic acid dimeglumine salt, Gadobutrol, Gadocoletic acid trisodium salt, Gadodenterate, Gadomelitol, Gadopentetate dimeglumine, Gadoterate meglumine, Gadoteridol, Gambogic acid, Gamendazole, Gamma-Linolenic Acid, Ganefromycin Alpha, Ganefromycin Beta, Ganglioside GM1, Ganoderic acid X, Garenoxacin mesilate, Gastrazole, Gatifloxacin, Gemfibrozil, Gemifloxacin mesilate, Gemopatrilat, Gilatide, Gimatecan, Giripladib, Glaspimod, Glucarolactam potassium, Gludopa, Glutathione Monoethyl Ester, Glutathione Monoisopropyl Ester, Glycine-proline-Melphalan, Glycopin, Glycyrrhizinic acid, Golotimod, Goodyeroside B, Goralatide, Grepafloxacin hydrochloride, GS-143, Haterumadioxin A, Haterumadioxin B, Helvecardin A, Helvecardin B, Heptelidic acid chlorohydrin, Hericenal A, Hericenal B, Hericenal C, Homoindanomycin, Hongoquercin A, Hongoquercin B, Human angiotensin II, Hyaluronate sodium, Hydrostatin A, Ibuprofen, Icatibant acetate, Icofungipen, Idrapril, Ifetroban, Ilepatril, Iloprost, Imidapril, Imidapril hydrochloride, Imiglitazar, Imipenem, Indanaprost (S), Indanomycin, Indeglitazar, Indobufen, Indole-3-propionic acid, Indometacin, Indomethacin trometamol, Indoxam, Indynaprost, Inogatran, Inosiplex, Iododiflunisal, Iodofiltic acid-[123I], Iodostearic Acid, Iralukast, Iralukast sodium, Isalsteine, Isobongkrekic acid, Isotretinoin, Itavastatin calcium, Itriglumide, Kaitocephalin, Kanglemycin A, Kapurimycin A1, Kapurimycin A3, Ketoprofen, Ketoprofen lysine, Ketorolac, Ketorolac tromethamine, Khafrefungin, Kijimicin, Kistamicin A, L-4-Oxalysine, Labradimil, Lamectacin, Lamifiban, Lanthiopeptin, Lapaquistat acetate, Larazotide acetate, Laropiprant, Latamoxef sodium, L-Chicoric acid, Lenapenem hydrochloride, Lenapenem hydrochloride hydrate, Levocabastine hydrochloride, Levocetirizine dihydrochloride, levo-Ciprofibrate, Levodopa, Levodopa 3-O-glucoside, Levodopa 4-O-glucoside, Levofloxacin, Levonadifloxacin arginine salt, L-Homothiocitrulline, Licofelone, Licorice-saponin C2, Lidorestat, Limaprost alfadex, Limazocic, Linoleic acid 18:2w6-cis,9-cis, Linotroban, Lintitript, Lipohexin, Lisinopril, Lithium succinate, Lithospermic acid B magnesium salt, Loloatin B, Lomefloxacin hydrochloride, Lometrexol, Longestin, Lonidamine, Loracarbef hydrate, Lorglumide, Lotrafiban, Loxiglumide, L-Simexonyl homocysteine, L-Thiocitrulline, Lubiprostone, Lumiracoxib, Lu-Tex bis(gluconate), Lysinated-betulonic acid, Lysine acetylsalicylate, Macrocarpin B, Madecassic acid, Maracenin A1, Maracenin A2, Maracenin B1, Maracenin B2, Maracenin C1, Maracenin C2, Maracenin D1, Maracenin D2, Marbofloxacin, Maslinic acid, Matristatin A1, Matristatin A2, Matteuorienate A, Matteuorienate B, Matteuorienate C, Mebrofenin, Meclinertant, Mefenamic acid, Melagatran, Memno-peptide A, Meptazinol-Val-carbamate, Meropenem, Mersacidin, Mesalazine, Metesind glucuronate, Methanobactin, Methotrexate, Methoxatin, Methyldopa, Methylenolactocin, Methylhomoindanomycin, Metiapril, Metirosine, Micacocidin A, Micacocidin B, Midafotel, Midoriamin, Milrinone Lactate, Minerval, Mipitroban, Mispyric acid, Mixanpril, Moenomycin A chloride bismuth salt, Moexipril hydrochloride, Moexiprilat, Mofezolac, Momordin Ic, Monamidocin, Monoethanolamine oleate, Montelukast sodium, Morphine Glucuronide, Moxifloxacin hydrochloride, Mumbaistatin, Mupirocin, Muraglitazar, Muraminomicin A, Muraminomicin B, Muraminomicin C, Muraminomicin D, Muraminomicin E1, Muraminomicin E2, Muraminomicin F, Muraminomicin G, Muraminomicin H, Muraminomicin I, Muraminomicin Z1, Muraminomicin Z2, Muraminomicin Z3, Muraminomicin Z4, Mureidomycin A, Mureidomycin B, Mureidomycin C, Mureidomycin D, Mureidomycin E, Mureidomycin F, Mureidomycins, Mycaperoxide A, Mycaperoxide B, Mycestericin E, Mycophenolic acid sodium salt, Myriceric acid A, Mytolbilin acid, Nadifloxacin, Nafagrel hydrochloride, Nafagrel hydrochloride hemihydrate, Nagstatin, Napirimus, Napsagatran, Napsamycin A, Napsamycin B, Napsamycin C, Napsamycin D, Nateglinide, Naveglitazar, Nebostinel, Nemonoxacin, Neu5Ac2en, Niacin, Niglizin, Nileprost beta-cyclodextrin clathrate, Nooglutil, Norfloxacin, Norfloxacin succinil, Obeticholic acid, Octacosamicin A, Octacosamicin B, O-Demethylchlorothricin, Ofloxacin, Olamufloxacin, Olamufloxacin mesilate, Olanzapine pamoate, Oleanolic acid, Olmesartan, Olopatadine Hydrochloride, Olsalazine sodium, Omapatrilat, Onnamide A, OPC-17083, Opiorphin, Orbifloxacin, Oreganic acid, Orienticin A, Orienticin B, Orienticin C, Orienticin D, Oritavancin, Orniplabin, Oseltamivir carboxylate, Ovothiol A, Ovothiol B, Ovothiol C, Oxaprozin, Oxeglitazar, Oxiglutatione sodium, Oxymorphone-Val-carbamate, Oxynor, Ozagrel hydrochloride, Ozenoxacin, Pactimibe, Padoporfin, Paeciloquinone B, Paeciloquinone D, Paldimycin B, Palovarotene, Panipenem, Parasin I, Parinaric acid, Paulomycin, Paulomycin A2, Paulomycin B, Paulomycin C, Paulomycin D, Paulomycin E, Paulomycin F, Pazufloxacin, Pazufloxacin mesilate, Pefloxacin, PEG-vancomycin, Pelagiomicin C, Peliglitazar, Pelitrexol, Pelretin, Penasterol, Penicillamine, Peramivir, Perindopril, PG-camptothecin, Phomallenic acid C, Phomoidride A, Phomoidride B, Phosphinic cyclocreatine, Phosphosalsalate, Physostigmine salicylate, Pibaxizine, Pidotimod, Piraxostat, Piretanide, Pirfenoxone, Pirprofen, Pivagabine, Pixantrone maleate, Plakotenin, Platencin, Platensimycin, Plevitrexed, Pluraflavin E, Plusbacin A1, Plusbacin A2, Plusbacin A3, Plusbacin A4, Plusbacin B1, Plusbacin B2, Plusbacin B3, Plusbacin B4, Polyalthidin, Pomisartan, Ponalrestat, Poststatin, PPI17-24, Pradimicin A, Pradimicin B, Pradimicin D, Pradimicin E, Pradimicin FA-1, Pradimicin FA-2, Pradimicin FL, Pradimicin FS ((+)-enantiomer), Pradimicin L, Pradimicin Q, Pradimicin S, Pradimicin T1, Pradimicin T2, Pradofloxacin, Pralatrexate, Pranoprofen, Prefolic A, Pregabalin, Premafloxacin, Premafloxacin hydrochloride, Prezatide copper acetate, Proamipide, Probenecid, Probestin, Procysteine, Proglumide, Propagermanium, Propofol hemisuccinate, Prostatin, Prostratin succinate, Protocatechuic acid, Protoporphyrin IX gallium(III) complex, Prulifloxacin, Prulifloxacin Hydrochloride, Prulifloxacin Mesylate, Pseudomycin A′, Pseudomycin B′, Pycnanthuquinone A, Pycnanthuquinone B, Pyloricidin B, Pyridazomycin, Pyrrolosporin A, Quiflapon Sodium, Quinapril hydrochloride, Quinlukast, Rafabegron, Ragaglitazar, Raltitrexed, Ramatroban, Ramipril, Raxofelast, Razupenem, Rebamipide bismuth citrate tetramethyledamine, Rebamipide bismuth L-tartrate tetramethyledamine, Repaglinide, Resobene, Reveromycin A, Rhododaurichromanic acid A, Ridogrel, Robenacoxib, Rocagloic acid, Rolafagrel, Romazarit, Romurtide, Rosaprostol sodium, Rosuvastatin calcium, Rosuvastatin sodium, Rufloxacin Gluconate, Rufloxacin hydrochloride, Rumycin 1, Rumycin 2, Salazopyridazin, Salcaprozic acid sodium salt, Salicylazobenzoic acid, S-Allylmercaptocaptopril, Salmisteine, Salvianolic acid L, Samixogrel, Sampatrilat, Sanfetrinem, Sanfetrinem sodium, Sapurimycin, Sarpogrelate hydrochloride, Saussureamine A, Saussureamine B, Saussureamine C, Saussureamine D, Saussureamine E, Scabronine G, Scopadulcic acid B, Securioside A, Securioside B, Selank, Semduramicin, Seocalcitol, Seratrodast, Serofendic acid, Sessiloside, Shepherdin, Sialosylcholesterol-Alpha Sodium Salt, Sitafloxacin hydrate, S-Nitrosocaptopril, S-Nitrosoglutathione, Sodelglitazar, Sodium cromoglycate, Sodium oxybate, Sofalcone, Solabegron hydrochloride, Sorbicillactone A, Sparfloxacin, Sphingofungin F, Spinorphin, Spirapril, Spiriprostil, Spiroglumide, Spiroximicin, Squalestatin I, Stachybocin A, Stachybocin B, Stachybocin C, Staplabin, Starrhizin, Sterenin D, Subtilopentadecanoic acid, Succinobucol, Sufotidine bismuth citrate, Sugammadex sodium, Sulfasalazine, Sulindac, Sulopenem, Sulukast, Sunflower trypsin inhibitor-1, Susalimod, Tafamidis meglumine, Tageflar, Talaglumetad hydrochloride, Talibegron, Talibegron hydrochloride, Talopterin, Taltobulin, Tamibarotene, Tanogitran, Tanomastat, TAP-doxorubicin, Tarenflurbil, Targinine, Tazarotenic Acid, Tebipenem, Teicoplanin-A2-1, Teicoplanin-A2-2, Teicoplanin-A2-3, Teicoplanin-A2-5, Telavancin hydrochloride, Telmesteine, Telmisartan, Temafloxacin hydrochloride, Temocapril hydrochloride, Temurtide, Tenosal, Terbogrel, Terestigmine tartrate, Terikalant fumarate, Tesaglitazar, Tetomilast, Tetradecylselenoacetic acid, Tetrafibricin, Tetragalloylquinic acid, Tetrahydroechinocandin B, Tetronothiodin, Tezampanel, Thermozymocidin, Thiazohalostatin, Thielavin G, Thielocin, Thielocin B3, Thiofoscarnet, Thioxamycin, Thrazarine, Thymic humoral factor gamma-2, Thymopentin, Tiagabine hydrochloride, Tibenelast, Ticolubant, Tilarginine hydrochloride, Tiliquinatine, Timodepressin, Tipelukast, Tiplasinin, Tirofiban hydrochloride, Tisartan, Tolfenamic acid, Tolmetin, Tolrestatin, Tomopenem, Tosufloxacin, Tosufloxacin Tosilate, Trandolapril, Trandolaprilat, Tranexamic acid, Tranilast, Treprostinil diethanolamine, Treprostinil sodium, Tretinoin, Triacetylshikimic acid, Trichomycin A, Triflusal, Trimexautide, Trimoprostil, Tripterin, Tropesin, Trovafloxacin, Trovafloxacin hydrate, Trovafloxacin hydrochloride mesylate, Trovafloxacin mesilate, Tubelactomicin A, Tuberactomycin D, Tuberactomycin E, Tubulysin A, Tubulysin B, Tubulysin C, Tucaresol, Tuftsin, Turbinaric acid, Tyroservatide, Ubenimex, Ulifloxacin, Uncarinic acid A, Uncarinic acid B, Unoprostone, Ursodeoxycholic acid, Ursolic acid phosphate, Utibapril, Utibaprilat, Vadimezan, Valonomycin A, Valproate Semisodium, Valproic acid, Valsartan, Vancomycin hydrochloride, Varespladib, Vebufloxacin, Vedaprofen, Veliflapon, Verlukast, Vinaxanthone, Viquidacin, Viranamycin-A, Viscosin, Vitilevuamide, Voreloxin, W Peptide, Xanthofulvin, Zabicipril Hydrochloride, Zabiciprilat Hydrochloride, Zabofloxacin hydrochloride, Zaltoprofen, Zanamivir, Zaragozic acid D3, Zenarestat, Zofenoprilat, Zofenoprilat arginine, Zolasartan, Zonampanel.

Suitable drugs with a phosphate group may be selected fromt the group consisting of Adenophostin A, Adenophostin B, Atrinositol, Buflomedil pyridoxalphosphate, Cytostatin, Fludarabine phosphate, Fosfluconazole, Fosfonochlorin, Fosfosal, Fosopamine, Fosquidone, Fostamatinib, Ganciclovir monophosphate, Genistein-7-phosphate, Hydroxyphoslactomycin B, Leustroducsin A, Leustroducsin B, Leustroducsin C, Leustroducsin H, Mangafodipir trisodium, Menadiol sodium diphosphate, Miproxifene phosphate, Monophosphoryl lipid A, Phospholine, Phosphosalsalate, Pneumocandin B0 2-phosphate, Tafluposide, Triciribine phosphate, Ursolic acid phosphate.

Suitable drugs with a thiol group may be selected fromt the group consisting of Acetylcysteine, Antileukinate, Argimesna, Bucillamine, Butixocort, Captopril, Dihydrolipoic acid, Gemopatrilat, Glutathione monoethyl ester, Glutathione monoisopropyl ester, Midoriamin, Omapatrilat, Ovothiol A, Ovothiol B, Ovothiol C, Penicillamine, Rebimastat, Shepherdin, Zofenoprilat, Zofenoprilat arginine.

In the following sections preferred embodiments of the present invention are described.

In a preferred embodiment, the carrier of formula (I) comprises a quaternary carbon, in particular a quaternary carbon of a branching core moiety B, wherein B is pentarythritol in bound form. Preferably, each A is independently a PEG-based polymeric chain terminally attached to the quaternary carbon of pentaerythritol via the —CH₂—O— moieties of pentaerythritol by a permanent covalent linkage, and the distal end of each moiety A is covalently bound to a moiety Hyp, each moiety Hyp is conjugated to m moieties L, either directly or indirectly through a moiety SP and which moieties L are each connected to a biologically active moiety D.

In one preferred embodiment, a moiety Hyp of formula (I) comprises, preferably consists of, branched polyamines comprising at least 2 amine groups. Preferably, such branched polyamine comprises one or more lysine residues in bound form. Preferably, each Hyp of formula (I) has a molecular weight of from 0.1 kDa to 4 kDa, in particular of from 0.2 to 2 kDa. In a preferred embodiment, n is 4 and each of the 4 moieties Hyp may independently consist of the same or different moieties Hyp. In a preferred embodiment, the 4 moieties Hyp are the same.

In a preferred embodiment, a moiety Hyp comprises, in particular consists of, between 1 and 32 lysines in bound form, preferably comprises, in particular consists of, 1, 3, 7 or 15 lysines in bound form, more preferably of 1, 3 or 7 lysines in bound form. Most preferably, Hyp comprises, in particular consists of, heptalysinyl.

Preferably, the carrier of formula (I) with n=4 has a molecular weight of from 1 kDa to 80 kDa, more preferably of from 1 kDa to 40 kDa and even more preferably of from 10 kDa to 40 kDa.

Preferred carrier moieties of formula (I) are selected from structures (i) to (iii):

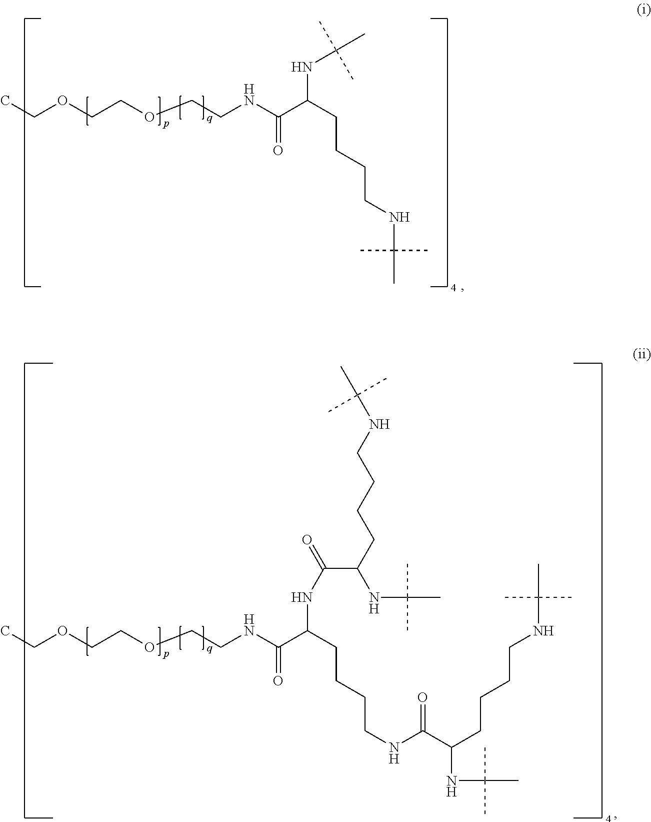

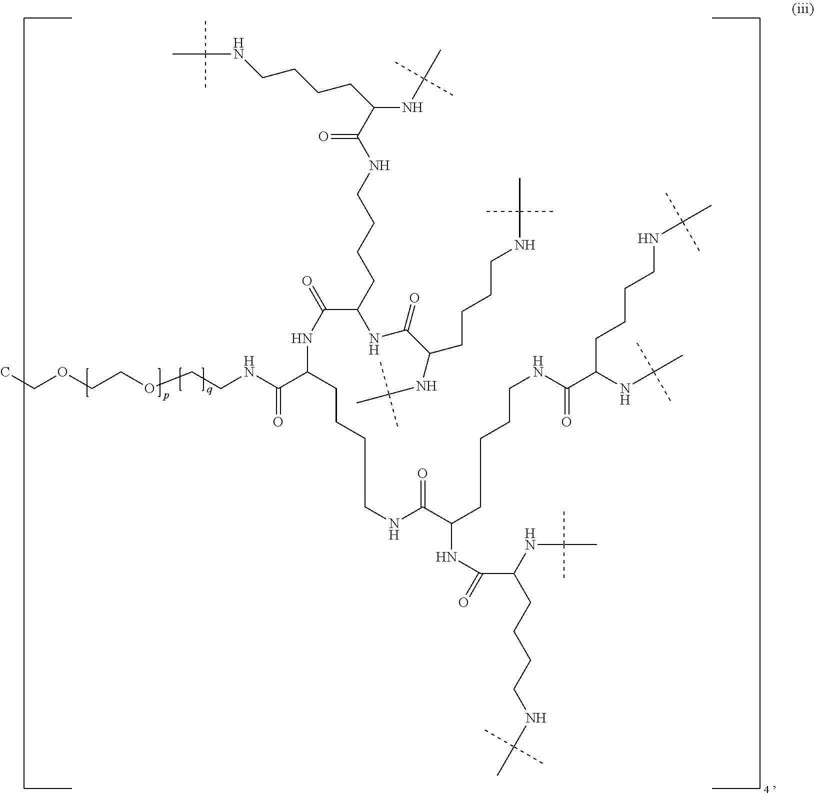

wherein
dashed lines indicate attachment to sub-structures —(SP)ₓ-L-D of formula (I),
p is an integer from 5 to 2000, preferably from 10 to 1000, more preferably from 10 to 500, and even more preferably from 100 to 500,
q is 1 or 2.

In a preferred embodiment, B is pentaerythritol.

Another subject of the present invention is a method for the synthesis of the water-soluble carrier-linked prodrug of formula (I) or a pharmaceutically acceptable salt thereof. Water-soluble carrier-linked prodrugs of formula (I) or precursors thereof invention may be prepared by known methods or in accordance with the reaction sequences described below. The starting materials used in the preparation (synthesis) of water-soluble carrier-linked prodrugs of formula (I) or precursors thereof are known or commercially available, or can be prepared by known methods or as described below.

A preferred starting material is a 4-arm-PEG amine reagent with the 4-arm-PEG amine reagent having a molecular weight ranging from 0.2 to 160 kDa. To such 4-arm-PEG amine reagent, lysine residues are coupled sequentially to form the carrier. It is understood that the lysines can be partially or fully protected by protective groups during the coupling steps and that also the final carrier may contain protective groups. A preferred building block is bis-boc lysine.

Alternatively, instead of sequential additions of lysine residues, a branched polylysine moiety may be assembled first and subsequently coupled to the 4-arm-PEG amine reagent. Such polylysine may be obtained by batch condensation or by means of sequential assembly using protected lysine building blocks.

For example it may be desirable to obtain such carrier carrying 32 amino groups, consequently seven lysines would be attached to each arm of a 4-arm-PEG amine.

In another embodiment, the PEG reagent may be a 4-arm-PEG-carboxylate. In this case the dendritic moieties may be generated from glutamic or aspartic acid, and the resulting carrier would carry a number of terminal carboxy groups.

Alternatively, instead of sequential additions of glutamic or aspartic acid residues, a branched poly-glutamate or poly-aspartate moiety may be assembled first and subsequently coupled to the 4-arm-PEG-carboxylate reagent. Such polyglutamate or -aspartate may be obtained by batch condensation or by means of sequential assembly using corresponding protected amino acid building blocks.

In yet another embodiment, an oligo- or polyglycerol may be converted into a corresponding poly-amine comprising a glycerol condensation product core. Such polyglycerol-derived poly-amine may be coupled to a 4-arm-PEG-carboxylate reagent to yield a suitable carrier. It is understood that carboxy groups may be activated to enhance their reactivity. For instance, the carboxy group may be converted into a chloride or an active ester.

It is also understood that all or a fraction of the carrier's reactive functional groups may be present in a free form, as salts or conjugated to protecting or activating groups. Due to practical reasons, the carrier reagent's number of branches per arm of the multi-arm, such as a 4-arm PEG reagent will be in a range of, for example 1 to 15, more preferably 1 to 7.

Functional groups of the carrier are then used for coupling linker reagents comprising suitable complementary functional groups to yield carrier-linker conjugate reagents. To such carrier-linker conjugate reagents are subsequently drugs coupled. Alternatively, a drug moiety may first be coupled to a linker reagent and subsequently, the biologically active moiety-linker reagent is coupled to the carrier.

Another aspect of the present invention is a pharmaceutical composition comprising the water-soluble carrier-linked prodrugs of formula (I) or a pharmaceutical salt thereof, and optionally one or more pharmaceutically acceptable excipients.

The pharmaceutical composition is further described in the following paragraphs.

The pharmaceutical composition comprising the water-soluble carrier-linked prodrug of formula (I) may be provided as a liquid composition or as a dry composition. Suitable methods of drying are, for example, spray-drying and lyophilization (freeze-drying). A preferred method of drying is lyophilization.

Preferably, the water-soluble carrier-linked prodrug of formula (I) is sufficiently dosed in the composition to provide a therapeutically and/or diagnostically effective amount of the biologically active moiety, in particular for at least one day in one application. More preferably, one application of the pharmaceutical composition comprising the water-soluble carrier-linked prodrug is sufficient for at least two days, such as three days, four days, five days, six days, or is sufficiently dosed for at least one week, such as for one week, two weeks, three weeks, four weeks, five weeks, six weeks, seven weeks, eight weeks, three months, four months, five months or six months.

A pharmaceutical composition comprising a water-soluble carrier-linked prodrug or a pharmaceutically acceptable salt thereof of formula (I) preferably comprises one or more excipients.

Excipients may be categorized as buffering agents, isotonicity modifiers, preservatives, stabilizers, anti-adsorption agents, oxidation protection agents, viscosifiers/viscosity enhancing agents, or other auxiliary agents. In some cases, these ingredients may have dual or triple functions. The pharmaceutical compositions of water-soluble carrier-linked prodrugs according to the present invention preferably comprise one or more excipients, selected from the groups consisting of:
(i) Buffering agents: physiologically tolerated buffers to maintain pH in a desired range, such as sodium phosphate, bicarbonate, succinate, histidine, citrate and acetate, sulphate, nitrate, chloride, pyruvate. Antacids such as Mg(OH)₂ or ZnCO₃ may be also used. Buffering capacity may be adjusted to match the conditions most sensitive to pH stability
(ii) Isotonicity modifiers: to minimize pain that can result from cell damage due to osmotic pressure differences at the injection depot. Glycerin and sodium chloride are examples. Effective concentrations can be determined by osmometry using an assumed osmolality of 285-315 mOsmol/kg for serum
(iii) Preservatives and/or antimicrobials: multidose parenteral preparations require the addition of preservatives at a sufficient concentration to minimize risk of patients becoming infected upon injection and corresponding regulatory requirements have been established. Typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride
(iv) Stabilizers: Stabilization is achieved by strengthening of the protein-stabilizing forces, by destabilization of the denatured state, or by direct binding of excipients to the protein. Stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives. In addition, oligomers or polymers such as cyclodextrins, dextran, dendrimers, PEG or PVP or protamine or HSA may be used
(v) Anti-adsorption agents: Mainly ionic or non-ionic surfactants or other proteins or soluble polymers are used to coat or adsorb competitively to the inner surface of the composition's or composition's container. Suitable surfactants are e.g., alkyl sulfates, such as ammonium lauryl sulfate and sodium lauryl sulfate, alkyl ether sulfates, such as sodium laureth sulfate and sodium myreth sulfate, sulfonates such as dioctyl sodium sulfosuccinates, perfluorooctanesulfonates, perfluorobutanesulfonates, alkyl benzene sulfonates, phosphates, such as alkyl aryl ether phosphates and alkyl ether phosphates, carboxylates, such as fatty acid salts (soaps) or sodium stearate, sodium lauroyl sarcosinate, perfluorononanoate, perfluorooctanoate, octenidine dihydrochloride, quaternary ammonium cations such as cetyl trimethylammonium bromide, cetyl trimethylammonium chloride, cetylpyridinium chloride, polyethoxylated tallow amine, benzalkonium chloride, benzethonium chloride, 5-bromo-5-nitor-1,3-dioxane, dimethyldioctadecylammonium chloride, dioctadecyldimethylammonium bromide, zwitterionics, such as 3-[(3-cholamidopropyl)dimethylammonio]-1-propanesulfonate, cocamidopropyl hydroxysultaine, amino acids, imino acids, cocamidopropyl betaine, lecithin, fatty alcohols, such as cetyl alcohol, stearyl alcohol, cetostearyl alcohol, oleyl alcohol, polyoxyethylene glycol alkyl ethers, such as octaethylene glycol monododecyl ether, pentaethylene glycol monododecyl ether, polyoxypropylene glycol alkyl ethers, glucoside alkyl ethers, such as decyl glucoside, lauryl glucoside, octyl glucoside, polyoxyethylene glycol octylphenol ethers such as Triton X-100, polyoxyethylene glycol alkylphenol ethers such as nonoxynol-9, glycerol alkyl esters such as glyceryl laurate, polyoxyethylene glycol sorbitan alkyl esters such as polysorbates, sorbitan alkyl esters, cocamide MEA and cocamide DEA, dodecyl dimethylamine oxide, block copolymers of polyethylene glycol and polypropylene glycol, such as poloxamers (Pluronic F-68), PEG dodecyl ether (Brij 35), polysorbate 20 and 80, other anti-absorption agents are dextran, polyethylene glycol, PEG-polyhistidine, BSA and HSA and gelatines. Chosen concentration and type of excipient depends on the effect to be avoided but typically a monolayer of surfactant is formed at the interface just above the CMC value
(vi) Lyo- and/or cryoprotectants: During freeze- or spray drying, excipients may counteract the destabilizing effects caused by hydrogen bond breaking and water removal. For this purpose sugars and polyols may be used but corresponding positive effects have also been observed for surfactants, amino acids, non-aqueous solvents, and other peptides. Trehalose is particulary efficient at reducing moisture-induced aggregation and also improves thermal stability potentially caused by exposure of protein hydrophobic groups to water. Mannitol and sucrose may also be used, either as sole lyo/cryoprotectant or in combination with each other where higher ratios of mannitol:sucrose are known to enhance physical stability of a lyophilized cake. Mannitol may also be combined with trehalose. Trehalose may also be combined with sorbitol or sorbitol used as the sole protectant. Starch or starch derivatives may also be used
(vii) Oxidation protection agents: antioxidants such as ascorbic acid, ectoine, methionine, glutathione, monothioglycerol, morin, polyethylenimine (PEI), propyl gallate, vitamin E, chelating agents such aus citric acid, EDTA, hexaphosphate, thioglycolic acid
(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue. A spreading agent such as but not limited to hyaluronidase temporarily decreases the viscosity of the extracellular matrix and promotes diffusion of injected drugs.
(ix) Other auxiliary agents: such as wetting agents, viscosity modifiers, antibiotics, hyaluronidase. Acids and bases such as hydrochloric acid and sodium hydroxide are auxiliary agents necessary for pH adjustment during manufacture.

In a general embodiment the pharmaceutical composition comprising the water-soluble carrier-linked prodrugs of formula (I) in either dry or liquid form may be provided as a single or multiple dose composition.

In one embodiment of the present invention, the liquid or dry pharmaceutical composition comprising the water-soluble carrier-linked prodrug is provided as a single dose, meaning that the container in which it is supplied contains one pharmaceutical dose in case of therapeutically active drugs.

Alternatively, in one embodiment, the liquid or dry pharmaceutical composition comprising the water-soluble carrier-linked prodrug is a multiple dose composition, meaning that the container in which it is supplied contains more than one therapeutic dose, i.e., a multiple dose composition contains at least 2 doses in case of therapeutically active drugs. Such multiple dose composition of water-soluble carrier-linked prodrug can either be used for different patients in need thereof or can be used for one patient, wherein the remaining doses are stored after the application of the first dose until needed.

In another aspect of the present invention the pharmaceutical composition is in a container. Suitable containers for liquid or dry compositions are, for example, syringes, vials, vials with stopper and seal, ampouls, and cartridges. In particular, the liquid or dry composition comprising the water-soluble carrier-linked prodrug of formula (I) is provided in a syringe. If the pharmaceutical composition comprising the water-soluble carrier-linked prodrug is a dry pharmaceutical composition the container preferably is a dual-chamber syringe. In such embodiment, said dry pharmaceutical composition is provided in a first chamber of the dual-chamber syringe and reconstitution solution is provided in the second chamber of the dual-chamber syringe.

Prior to applying the dry composition of water-soluble carrier-linked prodrug to a patient in need thereof, the dry composition is reconstituted. Reconstitution can take place in the container in which the dry composition of water-soluble carrier-linked prodrug is provided, such as in a vial, syringe, dual-chamber syringe, ampoule, and cartridge. Reconstitution is done by adding a predefined amount of reconstitution solution to the dry composition. Reconstitution solutions are sterile liquids, such as water or buffer, which may contain further additives, such as preservatives and/or antimicrobials, such as, for example, benzylalcohol and cresol. Preferably, the reconstitution solution is sterile water. When a dry composition is reconstituted, it is referred to as a “reconstituted pharmaceutical composition” or “reconstituted composition”.

An additional aspect of the present invention relates to the method of administration of a reconstituted or liquid pharmaceutical composition comprising the water-soluble carrier-linked prodrug of formula (I). The pharmaceutical composition comprising water-soluble carrier-linked prodrug may be administered by methods of inhalation, injection or infusion, including intradermal, subcutaneous, intramuscular, intravenous, intraosseous, and intraperitoneal. Preferably, the pharmaceutical composition comprising water-soluble carrier-linked prodrug is administered subcutaneously.

The preferred method of administration for dry pharmaceutical compositions comprising the water-soluble carrier-linked prodrugs of the present invention is via inhalation.

Therefore, in a preferred embodiment, the present invention relates to a water-soluble carrier-linked prodrug or a pharmaceutically acceptable salt thereof of formula (I) or a pharmaceutical composition of the present invention, for use as medicament for topical, enteral administration, parenteral administration, inhalation, injection, or infusion, intraarticular, intradermal, subcutaneous, intramuscular, intravenous, intraosseous, and intraperitoneal, intrathecal, intracapsular, intraorbital, intracardiac, transtracheal, subcuticular, intraarticular, subcapsular, subarachnoid, intraspinal, intraventricular or intrasternal administration.

Therefore, in another preferred embodiment, the present invention relates to a water-soluble carrier-linked prodrug or a pharmaceutically acceptable salt thereof of formula (I) or a pharmaceutical composition of the present invention, wherein such water-soluble carrier-linked prodrug or pharmaceutically acceptable salt thereof or pharmaceutical composition is suitable to be administered to a patient via topical, enteral or parenteral administration and by methods of external application, inhalation, injection or infusion, including intraarticular, intradermal, subcutaneous, intramuscular, intravenous, intraosseous, and intraperitoneal, intrathecal, intracapsular, intraorbital, intracardiac, transtracheal, subcuticular, intraarticular, subcapsular, subarachnoid, intraspinal, intraventricular and intrasternal application.

A further aspect is a method of preparing a reconstituted composition comprising a diagnostically and/or therapeutically effective amount of water-soluble carrier-linked prodrug of formula (I), and optionally one or more pharmaceutically acceptable excipients, the method comprising the step of
contacting the pharmaceutical composition comprising water-soluble carrier-linked prodrug of formula (I) with a reconstitution solution.

Another aspect is a reconstituted pharmaceutical composition comprising a diagnostically and/or therapeutically effective amount of the water-soluble carrier-linked prodrug of formula (I), and optionally one or more pharmaceutically acceptable excipients.

Another aspect of the present invention is the method of manufacturing a dry composition of water-soluble carrier-linked prodrug. In one embodiment, such dry composition is obtainable by
(i) admixing the water-soluble carrier-linked prodrug with one or more excipients,
(ii) transfering amounts equivalent to single or multiple doses into a suitable container,
(iii) drying the composition in said container, and
(iv) sealing the container.

Suitable containers are vials, syringes, dual-chamber syringes, ampoules, and cartridges.

Another aspect of the present invention is a kit of parts.

If the administration device is simply a hypodermic syringe then the kit may comprise the syringe, a needle and a container comprising the dry pharmaceutical composition of water-soluble carrier-linked prodrug suitable for use with the syringe and a second container comprising the reconstitution solution.

If the pharmaceutical composition is a liquid composition then the kit may comprise the syringe, a needle and a container comprising the liquid composition of water-soluble carrier-linked prodrug suitable for use with the syringe.

In more preferred embodiments, the injection device is other than a simple hypodermic syringe and so the separate container with reconstituted or liquid water-soluble carrier-linked prodrug is adapted to engage with the injection device such that in use the liquid composition in the container is in fluid connection with the outlet of the injection device. Examples of administration devices include but are not limited to hypodermic syringes and pen injector devices. Particularly preferred injection devices are the pen injectors in which case the container is a cartridge, preferably a disposable cartridge. Optionally, the kit of parts comprises a safety device for the needle which can be used to cap or cover the needle after use to prevent injury.

A preferred kit of parts comprises a needle and a container containing the composition according to the present invention and optionally further containing a reconstitution solution, the container being adapted for use with the needle. Preferably, the container is a dual-chamber syringe.

In another aspect, the invention provides a cartridge comprising a pharmaceutical composition of water-soluble carrier-linked prodrug as hereinbefore described for use with a pen injector device. The cartridge may contain a single dose or multiplicity of doses of the water-soluble carrier-linked prodrug.

Yet another aspect of the present invention is a water-soluble carrier-linked prodrug or a pharmaceutically acceptable salt thereof of formula (I) or a pharmaceutical composition of the present invention, for use as a medicament and/or diagnostic.

In another embodiment, the present invention relates to the use of a water-soluble carrier-linked prodrug of formula (I) or a pharmaceutically acceptable salt thereof, or a pharmaceutical composition of the present invention for the preparation of a medicament and/or diagnostic, in particular for the treatment and/or diagnosis of diseases.

It is understood, that the disease that can be treated and/or diagnosed a water-soluble carrier-linked prodrug of the present invention or a pharmaceutically acceptable salt thereof, or a pharmaceutical composition of the present invention depends on the active agent. A water-soluble carrier-linked prodrug with an active agent moiety which has anti-cancer activity, like Doxorubicin, is typically administered to a cancer patient. Analogously, a water-soluble carrier-linked prodrug with an active agent moiety which has anti-inflammatory activity, like aminosalicylic acid, is typically administered to a patient who suffers from an inflammatory disease, like rheumatoid arthritis, IBD or Morbus Crohn. Analogously, a water-soluble carrier-linked prodrug with an active agent moiety which has neurological activity is typically administered to a patient suffering from a neurological disease like Alzheimer's disease or Parkinson's disease. Analogously, a water-soluble carrier-linked prodrug with an active agent moiety which has anti-infective activity, like Gancyclovir, is typically administered to a patient suffering from a infectious disease like bacterial, viral, protozoal or fungal infection.

In case the water-soluble carrier-linked prodrugs according to the invention contain one or more acidic or basic groups, the invention also comprises their corresponding pharmaceutically or toxicologically acceptable salts, in particular their pharmaceutically utilizable salts. Thus, the water-soluble carrier-linked prodrugs according to the invention which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Water-soluble carrier-linked prodrugs according to the invention which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the water-soluble carrier-linked prodrugs according to the invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example by contacting these with an organic or inorganic acid or base in a solvent or dispersant, or by anion exchange or cation exchange with other salts. The present invention also includes all salts of the prodrugs which, owing to low physiological compatibility, are not directly suitable for use in pharmaceuticals but which can be used, for example, as intermediates for chemical reactions or for the preparation of pharmaceutically acceptable salts.

Yet another aspect of the present invention is a method of treating, controlling, delaying or preventing in a mammalian patient, preferably in a human, in need of the treatment of one or more conditions comprising administering to said patient a diagnostically and/or therapeutically effective amount of a water-soluble carrier-linked prodrug of the present invention or a pharmaceutical composition comprising the water-soluble carrier-linked prodrug of the present invention or a pharmaceutically acceptable salt thereof.

### OPERATIVE EXAMPLES

The subject matter of the present invention is elucidated in more detail below, using examples, without any intention that the subject matter of the invention should be confined to these exemplary embodiments.

### Materials and Methods

Pramipexole dihydrochloride was obtained from Carbone Scientific Co., Ltd., Wuhan, China. Amino 4-arm PEGs were obtained from JenKem Technology, Beijing, P. R. China. 2-Chlorotrityl chloride resin was obtained from Merck Biosciences GmbH, Schwalbach/Ts, Germany. Paliperidone was purchased from Carbon Scientific Co., Ltd, London, UK. All other chemicals were purchased from Sigma-ALDRICH Chemie GmbH, Taufkirchen, Germany.

Solid phase synthesis was performed in syringes equipped with polyethylene frits as reaction vessels.

### RP-HPLC Purification:

RP-HPLC was done on a 100×20 or a 100×40 mm C18 ReproSil-Pur 300 ODS-3 5 pm column (Dr. Maisch, Ammerbuch, Germany) connected to a Waters 600 HPLC System and Waters 2487 Absorbance detector. Linear gradients of solution A (0.1% TFA in H₂O) and solution B (0.1% TFA in acetonitrile or 0.1% TFA in 2/1 (v/v) methanol/isopropanol) were used. HPLC fractions containing product were lyophilized. Alternatively, if the HCl salt of the purified product was desired, TFA was replaced by 0.01% HCl (v/v, 37% HCl) in solution A and solution B.

### LC-MS Analytics:

Liquid chromatography-electronspray ionization mass spectrometry was performed on a Waters Acquity Ultra Performance LC instrument connected to a Thermo scientific LTQ Orbitrap Discovery instrument and spectra were, if necessary, interpreted by Thermo scientific software xcalibur. M/z signals corresponding to the most abundant isotope are given.

### Example 1

### Synthesis of Pramipexole Linker Building Block

### Synthesis of pramipexole(boc) 1a

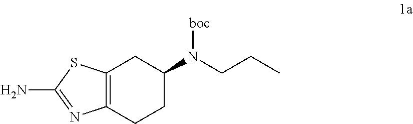

Pramipexole dihydrochloride (MW 284 g/mol, 400 mg, 1.41 mmol) and Di-tert-butyl dicarbonate (MW 218 g/mol, 307 mg, 1.41 mmol) were dissolved in DMSO (5 mL). DIEA (735 μL, 4.22 mmol) was added and solution was stirred for 3 h at RT. 1a was purified by RP-HPLC.

Yield: 422 mg (0.99 mmol, TFA salt).

MS: m/z 312.2=[M+H]⁺ (MW calculated=311.5 g/mol).

### Synthesis of Fmoc-Gly-pramipexole(boc) 1b

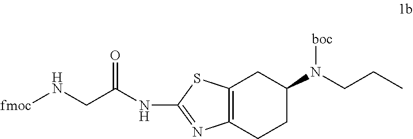

Pramipexole(boc) 1a (MW 311, 5 g/mol, 50 mg, 0.118 mmol), Fmoc-Gly-OH (MW 297 g/mol, 52 mg, 0.176 mmol) and PyBOP (104 mg, 0.200 mmol) were dissolved in DMSO (200 μL). DIEA (90 μL, 0.517 mmol) was added and the solution was agitated for 15 h. Fmoc-protected intermediate 1b was purified by RP-HPLC.

Yield: 72 mg (0.12 mmol).

MS: m/z 591.3=[M+H]⁺ (MW calculated=590.8 g/mol).

### Synthesis of H-Gly-pramipexole(boc) 1c

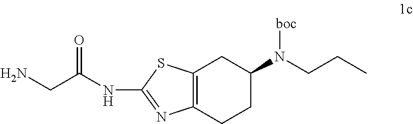

Compound 1b was dissolved in piperidine/DBU/DMF (2/2/96) and stirred for 30 min at RT.

Product 1c was purified by RP-HPLC.

Yield: 30.6 mg (0.06 mmol, TFA salt).

MS: m/z 369.2=[M+H]⁺ (MW calculated=368.5 g/mol).

### Synthesis of Pramipexole Linker Building Block 1d

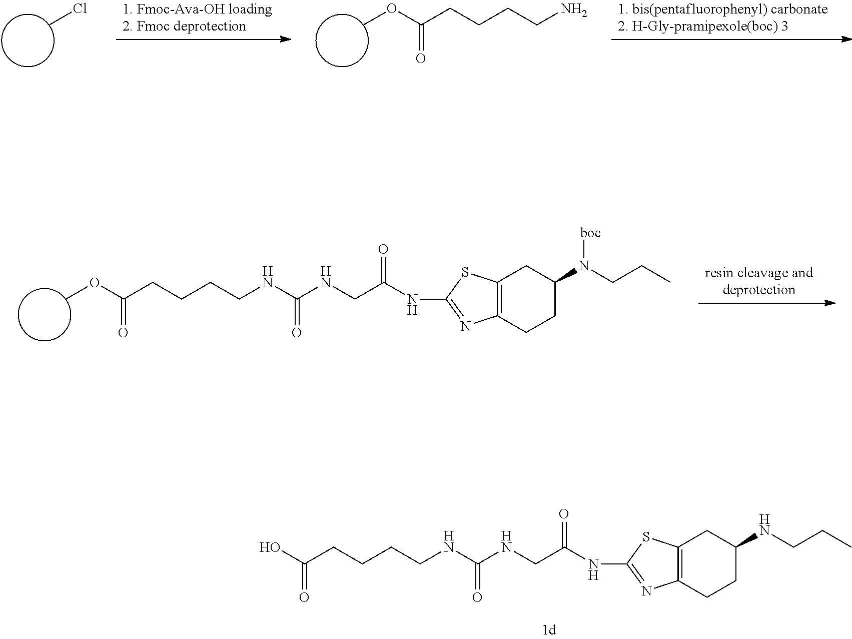

2-Chlorotrityl chloride resin (1.0 mmol/g, 420 mg, 0.42 mmol) was loaded with Fmoc-4-aminovaleric acid according to manufacturers instruction. For Fmoc removal, the resin was agitated with 2/2/96 (v/v/v) piperidine/DBU/DMF (two times, 10 min each) at RT and washed with DMF (ten times).

Synthesis of urea: Resin was washed with DCM (10 times) and reacted with bis(pentafluorophenyl) carbonate (MW 394 g/mol, 414 mg, 1.05 mmol) and DIEA (365 μL, 2.1 mmol) in DCM at RT for 45 min. Resin was washed with DCM (5 times) and DMF (10 times) and reacted with 1c (MW 482.5 g/mol, 203 mg, 0.42 mmol) and 2.5 eq DIEA (183 μL, 1.05 mmol) in DMF for 75 min at RT. Resin was washed with DMF (10 times).

Cleavage from resin and boc deprotection: Resin was washed with DCM (10 times) and treated three times for 30 minutes with 7/3 (v/v) DCM/HFIP. Eluates were combined and volatiles were removed under reduced pressure. Residue was incubated in TFA for 10 min at RT. TFA was removed under a stream of nitrogen and 1d was purified by RP-HPLC.

Yield: 160 mg (0.258 mmol, HCl salt).

MS: m/z 412.2=[M+H]⁺ (MW calculated=411.5 g/mol).

### Example 2

### Synthesis of 4-Arm PEG Trilysine Carrier

### Synthesis of 4-arm PEG lysine(boc) 2a

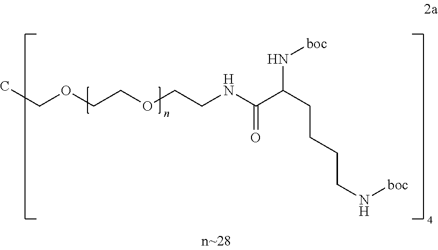

4-Arm-PEG5000 tetraamine (MW ca. 5200 g/mol, 5.20 g, 1.00 mmol, HCl salt) was dissolved in 20 mL of DMSO (anhydrous). Boc-Lys(Boc)-OH (2.17 g, 6.25 mmol) in 5 mL of DMSO (anhydrous), EDC HCl (1.15 g, 6.00 mmol), HOBt.H₂O (0.96 g, 6.25 mmol), and collidine (5.20 mL, 40 mmol) were added. The reaction mixture was stirred for 30 min at RT. The reaction mixture was diluted with 1200 mL of dichloromethane and washed with 600 mL of 0.1 N H₂SO₄ (2×), brine (1×), 0.1 M NaOH (2×), and 1/1 (v/v) brine/water (4×). Aqueous layers were reextracted with 500 mL of DCM. Organic phases were dried over Na₂SO₄, filtered and evaporated to give 6.3 g of crude product 2a as colorless oil. Compound 2a was purified by RP-HPLC.

Yield 3.85 g (59%) colorless glassy product 2a.

MS: m/z 1294.4=[M+5H]⁵⁺ (m/z of [M+5H]⁵⁺ calculated for a 4-Arm-PEG containing a total of 107 ethylene glycol units=1294.6).

### Synthesis of 4-Arm PEG Lysine 2b

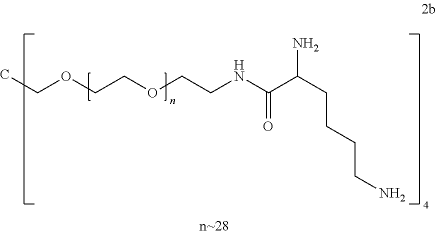

Compound 2b was obtained by stirring 3.40 g of compound 2a (0.521 mmol) in 5 mL of methanol and 9 mL of 4 N HCl in dioxane at RT for 15 min. Volatiles were removed in vacuo. The product was used in the next step without further purification.

MS: m/z 1151.9=[M+5H]⁵⁺ (m/z calculated=1152.0).

### Synthesis of 4-arm PEG trilysine(boc) 2c

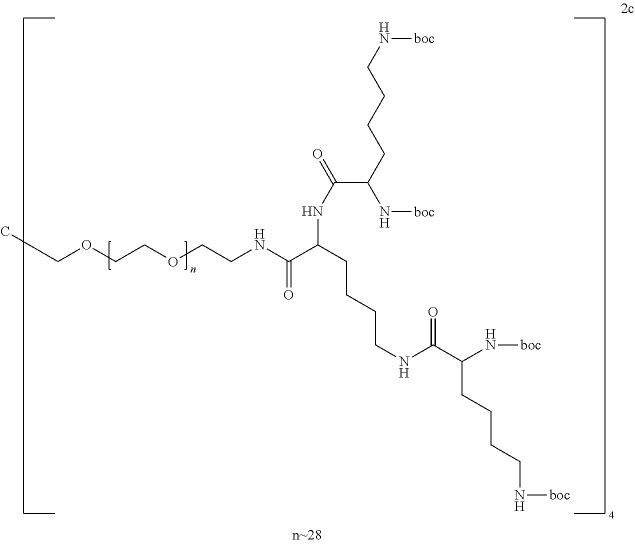

For synthesis of compound 2c, 3.26 g of compound 2b (0.54 mmol) were dissolved in 15 mL of DMSO (anhydrous). 2.99 g Boc-Lys(Boc)-OH (8.64 mmol) in 15 mL DMSO (anhydrous), 1.55 g EDC HCl (8.1 mmol), 1.24 g HOBt.H₂O (8.1 mmol), and 5.62 mL of collidine (43 mmol) were added. The reaction mixture was stirred for 30 min at RT.

Reaction mixture was diluted with 800 mL DCM and washed with 400 mL of 0.1 N H₂SO₄ (2×), brine (1×), 0.1 M NaOH (2×), and 1/1 (v/v) brine/water (4×). Aqueous layers were reextracted with 800 mL of DCM. Organic phases were dried with Na₂SO₄, filtered and evaporated to give a glassy crude product.

Product was dissolved in DCM and precipitated with cooled (−18° C.) diethyl ether. This procedure was repeated twice and the precipitate was dried in vacuo.

Yield: 4.01 g (89%) colorless glassy product 2c, which was used in the next step without further purification.

MS: m/z 1405.4=[M+6H]⁶⁺ (m/z calculated=1405.4).

### Synthesis of 4-arm PEG trilysine 2d

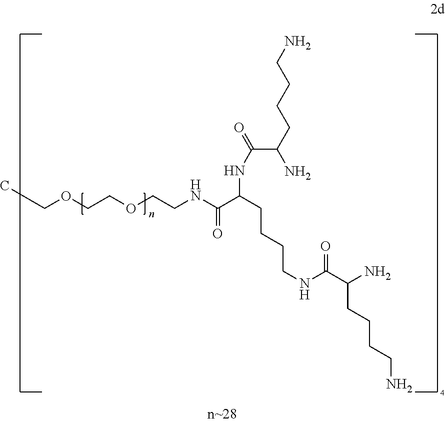

Compound 2d was obtained by stirring a solution of compound 2c (3.96 g, 0.47 mmol) in 7 mL of methanol and 20 mL of 4 N HCl in dioxane at RT for 15 min. Volatiles were removed in vacuo. The product was used in the next step without further purification.

MS: m/z 969.6=[M+7H]⁷⁺ (m/z calculated=969.7).

### Example 3

### Synthesis of carrier linked prodrug 4-arm PEG trilysine tetrapramipexole 3

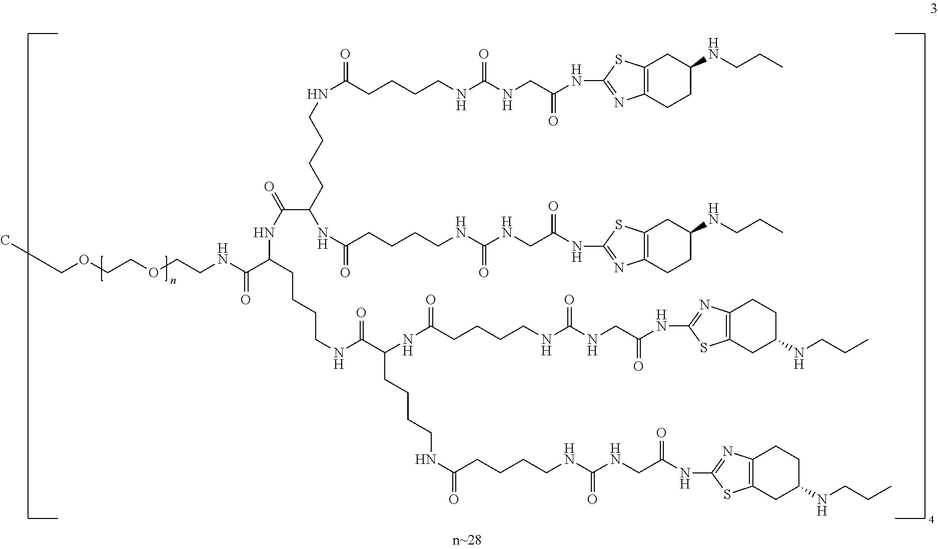

2d (MW approx 6500 g/mol, 20 mg, 3.1 μmol) in DMF (1 mL) is reacted with compound 1d (MW 448 g/mol, 90 mg 0.2 mmol), PyBOP (MW 520 g/mol, 105 mg, 0.2 mmol) and collidine (132 μL, 1.0 mmol) for 3 h at RT. 3 is purified by means of RP-HPLC.

### Example 4

### Synthesis of Branched Paliperidone Building Block

### Synthesis of Intermediate 4a

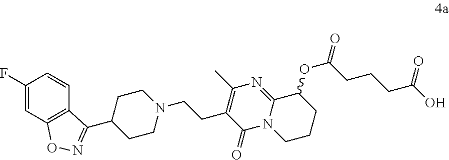 [SMILES: Cc1nc2n(c(=O)c1CCN1CCC(c3noc4cc(F)ccc34)CC1)CCCC2OC(=O)CCCC(=O)O]

5.35 g glutaric anhydride and 2.84 mL pyridine were added to a solution of 2.00 g paliperidone in 30 mL DCM (dry, mol. sieve). The reaction mixture was allowed to stir for 3 d at RT. Volatiles were removed and the resulting mixture was diluted with ACN/water 1/1 and acidified with acetic acid until pH reached about 4. 4a was purified by RP-HPLC.

Yield: 1.60 g (2.77 mmol, 60%, HCl salt).

MS: m/z 541.2=[M+H]⁺ (MW calculated=540.7)

### Synthesis of Intermediate 4b

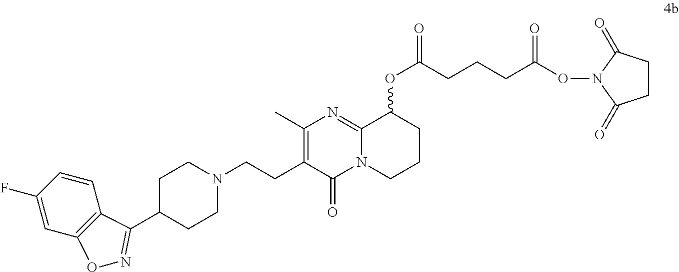 [SMILES: Cc1nc2n(c(=O)c1CCN1CCC(c3noc4cc(F)ccc34)CC1)CCCC2OC(=O)CCCC(=O)ON1C(=O)CCC1=O]

4a (1.50 g, 2.77 mmol) was dissolved in 40 mL DCM (dry, mol. sieve). DCC (1.72 g, 8.32 mmol), N-hydroxy succinimide (1.60 g, 13.87 mmol) and a catalytic amount of DMAP was added and mixture was stirred for 3 h at RT. Precipitate was filtered off and the solvent was removed under reduced pressure. Residue was dissolved with ACN/water 1/1 and acidified with acetic acid until pH reached about 4. 4b was purified by RP-HPLC.

Yield: 1.25 g (TFA salt, 1.66 mmol, 60%).

MS: m/z 638.25=[M+H]⁺ (MW calculated=637.67)

### Synthesis of Intermediate 4c

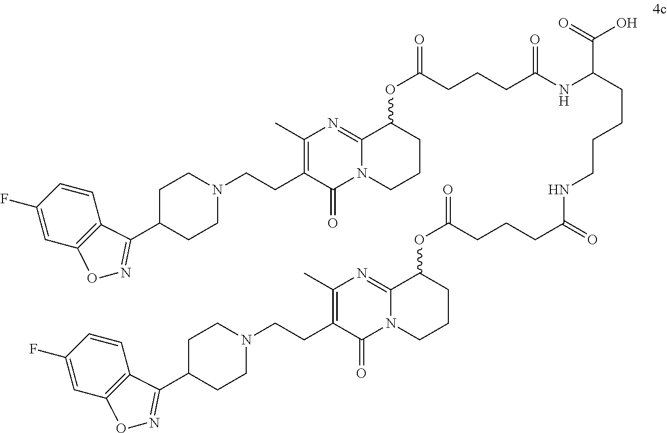 [SMILES: Cc1nc2n(c(=O)c1CCN1CCC(c3noc4cc(F)ccc34)CC1)CCCC2OC(=O)CCCC(=O)NCCCCC(NC(=O)CCCC(=O)OC1CCCn2c1nc(C)c(CCN1CCC(c3noc4cc(F)ccc34)CC1)c2=O)C(=O)O]

A solution of lysine (19 mg, 0.13 mmol) in 2.5 mL 0.5 M sodium borate buffer pH 8.5 was given to a solution of 4b (TFA salt, 300 mg, 0.40 mmol) in 5 mL DMSO. Mixture was stirred for 60 min at RT. Solution was acidified with acetic acid to a pH of approx. 4 and diluted with water and acetonitrile. 4c was purified by RP-HPLC.

Yield: 125 mg (HCl salt, 0.10 mmol, 74%).

MS: m/z 1191.55=[M+H]⁺ (MW calculated=1191.35)

### Synthesis of Intermediate 4d

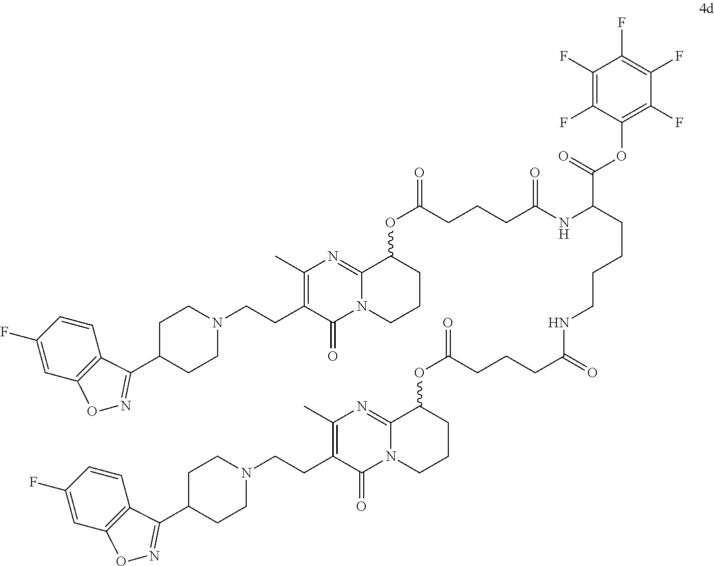 [SMILES: Cc1nc2n(c(=O)c1CCN1CCC(c3noc4cc(F)ccc34)CC1)CCCC2OC(=O)CCCC(=O)NCCCCC(NC(=O)CCCC(=O)OC1CCCn2c1nc(C)c(CCN1CCC(c3noc4cc(F)ccc34)CC1)c2=O)C(=O)Oc1c(F)c(F)c(F)c(F)c1F]

4c (bis HCl salt, 196 mg, 0.155 mmol) was dissolved in 12 mL DCM (anhydrous, mol. sieve). Bis(pentafluorophenyl) carbonate (MW 394 g/mol, 122 mg, 0.310 mmol) and sym-collidine (205 μL, 1.55 mmol) were added and mixture was stirred for 16 h at RT. Product was precipitated from reaction mixture by adding 30 mL MTBE (puriss., p.a.; >99.5%) and separated by centrifugation. Precipitate was redissolved in DCM and precipitation procedure was repeated. Precipitate was redissolved in DCM and volatiles were removed in vacuo (waterbath at 20° C.). Product 4d was dried by means of lyophilizer.

Yield: 185 mg (88%)

MS: m/z 1357.52=[M+H]⁺ (MW calculated=1357.40)

Pfp ester of 4d is partially hydrolyzed under LCMS conditions. A purity of 95% (LCMS, 215 nm) was confirmed after derivatization of 1d with 1-dodecylamine. For derivatization purpose 0.1 mg 4d is reacted with 0.3 mg 1-dodecylamine for 5 min at RT in DCM and analyzed by means of LCMS.

### Example 5

### Synthesis of carrier linked prodrug 4-arm PEG 20 kDa-octapaliperidone

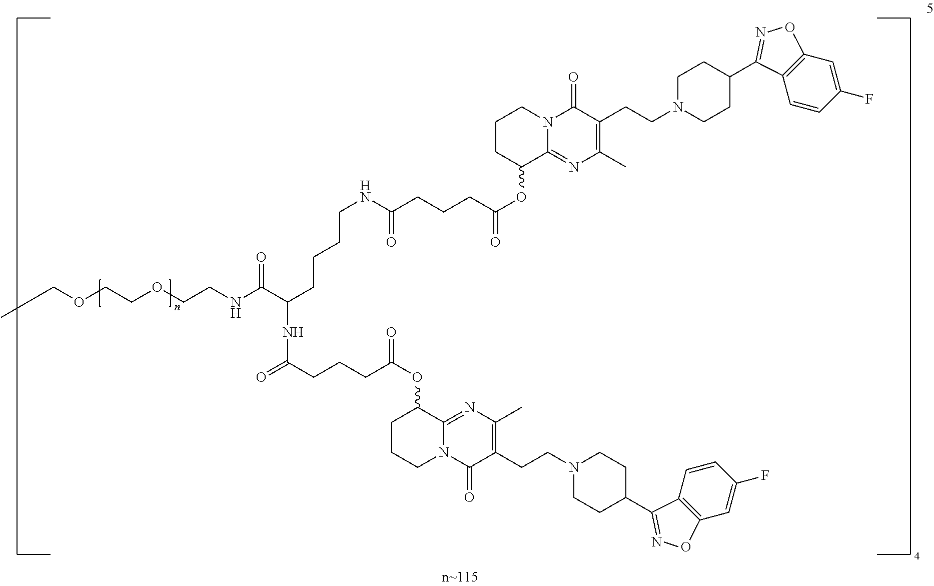

Amino 4-Arm PEG 20 kDa (MW 21 kDa, 60 mg, 2.9 μmol) was reacted with intermediate 4d (32 mg, 23 μmol) in ACN (1.5 mL, anhydrous, mol. sieve) and DIEA (8 μL, 47 μmol) under stirring for 16 h at RT. Mixture was quenched by addition of 1-dodecylamine (2.5 mg), acidified with acetic acid and diluted with water, followed by purification of compound 5 by RP-HPLC (main peak, 215 nm). Combined HPLC fractions (40 mL) were mixed with water (30 mL) and 0.5 M sodium phosphate pH 7.4 (4 mL). The mixture was extracted with DCM (25 mL, 3×) and combined organic phases were dried over Na₂SO₄ and evaporated under reduced pressure. Yield: 54 mg

Compound 5 proved to be a uniform material according to UPLC analytics (Waters BEH300 C18 column, 2.1×50 mm, 1.7 pm particle size, flow 0.25 mL/min, linear gradient 0-70% B in 4 min, mobile phase A: 0.05% TFA in water, mobile phase B: 0.04% TFA in acetonitrile), eluting at 3.60 min.

### Example 6

### Drug Release Kinetics from PEG Conjugate 5

Conjugate 5 (1.8 mg) was dissolved in acetonitrile (100 μl) and diluted with pH 7.4 buffer (60 mM sodium phosphate, 3 mM EDTA, 0.01% Tween-20, 1.4 mL). Sample was incubated at 37° C. At various time points aliquots were analyzed by UPLC and the amount of released paliperidone was plotted against time. Drug release was found to follow first order kinetics. Curve fitting software was used to determine half life time of drug release from the respective conjugate. A paliperidone release half life time of 5.5 d was obtained.

### ABBREVIATIONS

ACN acetonitrile

Boc t-butyloxycarbonyl

DCC N,N′-dicyclohexylcarbodiimide

DCM dichloromethane

DIEA diisopropylethylamine

DMAP dimethylamino-pyridine

DMF N,N-dimethylformamide

DMSO dimethylsulfoxide

EDC 1-ethyl-3-(3-dimethylaminopropyl) carbodiimide

eq stoichiometric equivalent

HOBt hydroxybenzotriazole

LCMS mass spectrometry-coupled liquid chromatography

MS mass spectrum

MTBE methyl-tert.-butylether

MW molecular mass

NMP N-methyl-2-pyrrolidone

PEG poly(ethylene glycol)

PyBOP benzotriazol-1-yl-oxytripyrrolidinophosphonium hexafluorophosphate

RP-HPLC reversed-phase high performance liquid chromatography

RT room temperature

TFA trifluoroacetic acid

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

1-13. (canceled)

14. A water-soluble carrier-linked prodrug of formula (I), or a pharmaceutically acceptable salt thereof, 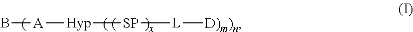
wherein
B, A and Hyp form a carrier moiety, and
wherein
B is a branching core,
each A is independently a poly(ethylene glycol)-based polymeric chain,
each Hyp is independently a branched moiety,
each SP is independently a spacer moiety,
each L is independently a reversible prodrug linker moiety,
each D is independently a biologically active moiety,
each x is independently 0 or 1,
each m is independently an integer of from 2 to 64, and
n is an integer from 3 to 32.

15. The water-soluble carrier-linked prodrug, or a pharmaceutically acceptable salt thereof, as claimed in claim 14, wherein m is an integer from 2 to 32.

16. The water-soluble carrier-linked prodrug, or a pharmaceutically acceptable salt thereof, as claimed in claim 15, wherein m is 2, 3, 4, 5, 6, 7, or 8.

17. The water-soluble carrier-linked prodrug, or a pharmaceutically acceptable salt thereof, as claimed in claim 14, wherein n is an integer from 3 to 16.

18. The water-soluble carrier-linked prodrug, or a pharmaceutically acceptable salt thereof, as claimed in claim 17, wherein n is 4.

19. The water-soluble carrier-linked prodrug, or a pharmaceutically acceptable salt thereof, as claimed in claim 14, wherein each A independently comprises the formula
X3-(CH₂)ₙ₁—(OCH₂CH₂)ₚ—O—(CH₂)ₙ₂—X2,
wherein
n1 and n2 are independently 1, 2, 3, or 4;
p is an integer from 5 to 2000;
X3 is a chemical bond or linkage group covalently linked to B; and
X2 is a chemical bond or linkage group covalently linked to Hyp.

20. The water-soluble carrier-linked prodrug, or a pharmaceutically acceptable salt thereof, as claimed in claim 19, wherein n1 and n2 are independently 2 or 3.

21. The water-soluble carrier-linked prodrug, or a pharmaceutically acceptable salt thereof, as claimed in claim 19, wherein p is an integer from 100 to 1000.

22. The water-soluble carrier-linked prodrug, or a pharmaceutically acceptable salt thereof, as claimed in claim 14, wherein each Hyp independently comprises a moiety selected from the group consisting of:
a polyalcohol in bound form comprising at least 2 hydroxyl groups;
a polyamine in bound form comprising at least 2 amine groups; and
a polycarboxylate in bound form comprising at least 2 carboxylate groups.

23. The water-soluble carrier-linked prodrug, or a pharmaceutically acceptable salt thereof, as claimed in claim 22, wherein the polyalcohol is selected from the group consisting of:
glycerol, pentaerythritol, dipentaerythritol, tripentaerythritol, hexaglycerine, sucrose, sorbitol, fructose, mannitol, glucose, cellulose, amyloses, starches, hydroxyalkyl starches, polyvinylalcohols, dextranes, and hyualuronans.

24. The water-soluble carrier-linked prodrug, or a pharmaceutically acceptable salt thereof, as claimed in claim 22, wherein the polyamine is selected from the group consisting of:
ornithine, diornithine, triornithine, tetraornithine, pentaornithine, hexaornithine, heptaornithine, octaornithine, nonaornithine, decaornithine, undecaornithine, dodecaornithine, tridecaornithine, tetradecaornithine, pentadecaornithine, hexadecaornithine, heptadecaornithine, octadecaornithine, nonadecaornithine, diaminobutyric acid, di(diaminobutyric acid), tri(diaminobutyric acid), tetra(diaminobutyric acid), penta(diaminobutyric acid), hexa(diaminobutyric acid), hepta(diaminobutyric acid), octa(diaminobutyric acid), nona(diaminobutyric acid), deca(diaminobutyric acid), undeca(diaminobutyric acid), dodeca(diaminobutyric acid), trideca(diaminobutyric acid), tetradeca(diaminobutyric acid), pentadeca(diaminobutyric acid), hexadeca(diaminobutyric acid), heptadeca(diaminobutyric acid), octadeca(diaminobutyric acid), nonadeca(diaminobutyric acid), lysine, dilysine, trilysine, tetralysine, pentalysine, hexalysine, heptalysine, octalysine, nonalysine, decalysine, undecalysine, dodecalysine, tridecalysine, tetradecalysine, pentadecalysine, hexadecalysine, heptadecalysine, octadecalysine, nonadecalysine, oligolysines, triornithine, tetraornithine, pentaornithine, hexaornithine, heptaornithine, octaornithine, nonaornithine, decaornithine, undecaornithine, dodecaornithine, tridecaornithine, tetradecaornithine, pentadecaornithine, hexadecaornithine, heptadecaornithine, octadecaornithine, nonadecaornithine, tridiaminobutyric acid, tetradiaminobutyric acid, pentadiaminobutyric acid, hexadiaminobutyric acid, heptadiaminobutyric acid, octadiaminobutyric acid, nonadiaminobutyric acid, decadiaminobutyric acid, undecadiaminobutyric acid, dodecadiaminobutyric acid, tridecadiaminobutyric acid, tetradecadiaminobutyric acid, pentadecadiaminobutyric acid, hexadecadiaminobutyric acid, heptadecadiaminobutyric acid, octadecadiaminobutyric acid, nonadecadiaminobutyric acid.

25. The water-soluble carrier-linked prodrug, or a pharmaceutically acceptable salt thereof, as claimed in claim 22, wherein the polycarboxylate is selected from the group consisting of:
di(glutamic acid), tri(glutamic acid), tetra(glutamic acid), penta(glutamic acid), hexa(glutamic acid), hepta(glutamic acid), octa(glutamic acid), nona(glutamic acid), deca(glutamic acid), undeca(glutamic acid), dodeca(glutamic acid), trideca(glutamic acid), tetradeca(glutamic acid), pentadeca(glutamic acid), hexadeca(glutamic acid), heptadeca(glutamic acid), octadeca(glutamic acid), nonadeca(glutamic acid), di(aspartic acid), tri(aspartic acid), tetra(aspartic acid), penta(aspartic acid), hexa(aspartic acid), hepta(aspartic acid), octa(aspartic acid), nona(aspartic acid), deca(aspartic acid), undeca(aspartic acid), dodeca(aspartic acid), trideca(aspartic acid), tetradeca(aspartic acid), pentadeca(aspartic acid), hexadeca(aspartic acid), heptadeca(aspartic acid), octadeca(aspartic acid), nonadeca(aspartic acid), polyethyleneimines, and polyvinylamines.

26. The water-soluble carrier-linked prodrug, or a pharmaceutically acceptable salt thereof, as claimed in claim 14, wherein Hyp has a molecular weight of from 0.1 to 4 kDa.

27. The water-soluble carrier-linked prodrug, or a pharmaceutically acceptable salt thereof, as claimed in claim 14, wherein Hyp has a molecular weight of from 0.2 to 2 kDa.

28. The water-soluble carrier-linked prodrug as claimed in claim 14, wherein B comprises a moiety selected from the group consisting of:
a polyalcohol in bound form comprising at least 2 hydroxyl groups; and
a polyamine in bound form comprising at least 2 amine groups.

29. The water-soluable carrier-linked prodrug as claimed in claim 28, wherein the polyalcohol is selected from the group consisting of:
glycerol, pentaerythritol, dipentaerythritol, tripentaerythritol, hexaglycerine, sucrose, sorbitol, fructose, mannitol, glucose, cellulose, amyloses, starches, hydroxyalkyl starches, polyvinylalcohols, dextranes, and hyualuronans.

30. The water-soluable carrier-linked prodrug as claimed in claim 28, wherein the polyamine is selected from the group consisting of:
ornithine, diornithine, triornithine, tetraornithine, pentaornithine, hexaornithine, heptaornithine, octaornithine, nonaornithine, decaornithine, undecaornithine, dodecaornithine, tridecaornithine, tetradecaornithine, pentadecaornithine, hexadecaornithine, heptadecaornithine, octadecaornithine, nonadecaornithine, diaminobutyric acid, di(diaminobutyric acid), tri(diaminobutyric acid), tetra(diaminobutyric acid), penta(diaminobutyric acid), hexa(diaminobutyric acid), hepta(diaminobutyric acid), octa(diaminobutyric acid), nona(diaminobutyric acid), deca(diaminobutyric acid), undeca(diaminobutyric acid), dodeca(diaminobutyric acid), trideca(diaminobutyric acid), tetradeca(diaminobutyric acid), pentadeca(diaminobutyric acid), hexadeca(diaminobutyric acid), heptadeca(diaminobutyric acid), octadeca(diaminobutyric acid), nonadeca(diaminobutyric acid), lysine, dilysine, trilysine, tetralysine, pentalysine, hexalysine, heptalysine, octalysine, nonalysine, decalysine, undecalysine, dodecalysine, tridecalysine, tetradecalysine, pentadecalysine, hexadecalysine, heptadecalysine, octadecalysine, nonadecalysine, oligolysines, polyethyleneimines, and polyvinylamines.

31. The water-soluble carrier-linked prodrug or a pharmaceutically acceptable salt thereof, as claimed in claim 14, wherein B comprises pentaerythritol.

32. The water-soluble carrier-linked prodrug, or a pharmaceutically acceptable salt thereof, as claimed in claim 14, wherein the carrier moiety is selected from one of structures (i) to (iii): 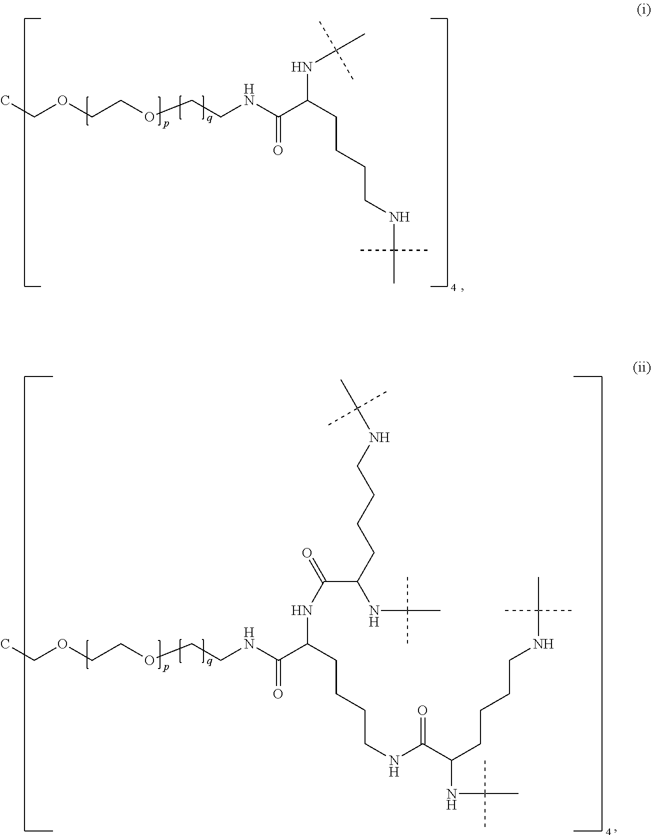 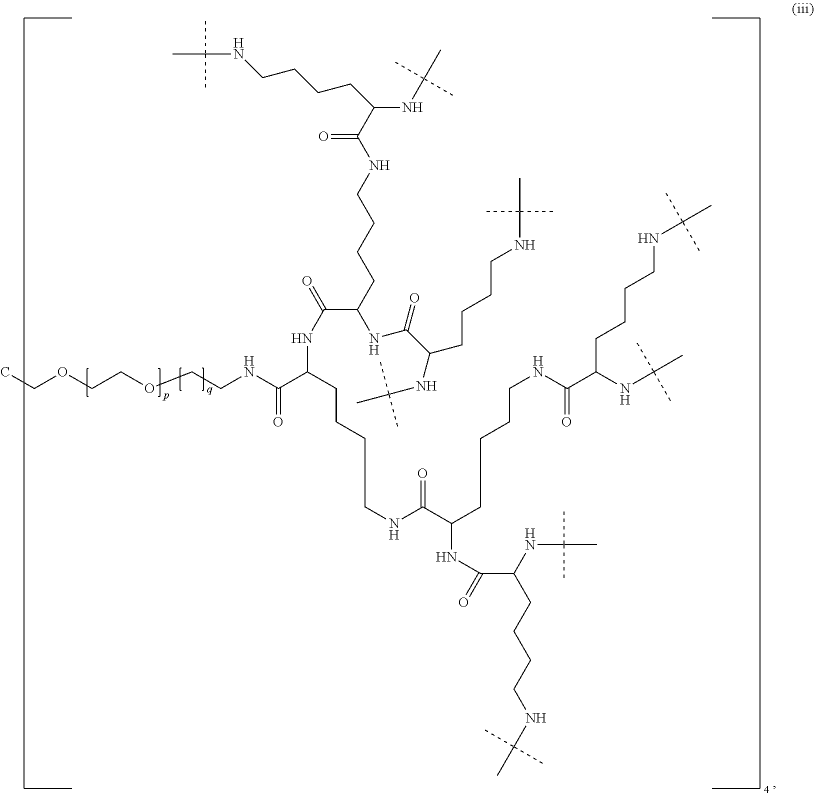
wherein
dashed lines indicate attachment to sub-structures —(SP)ₓ-L-D of formula (I);
p is an integer from 5 to 2000; and
q is 1 or 2.

33. The water-soluable carrier-linked prodrug, or a pharmaceutically acceptable salt thereof, as claimed in claim 32, wherein p is an integer from 100 to 500.

34. A pharmaceutical composition comprising the water-soluble carrier-linked prodrug, or a pharmaceutically acceptable salt thereof, as claimed in claim 14, further comprising one or more pharmaceutically acceptable excipients.

35. A method of treating, controlling, delaying or preventing in a mammalian patient in need of the treatment of one or more conditions comprising administering to the patient a diagnostically and/or therapeutically effective amount of the water-soluble protein carrier-linked prodrug or a pharmaceutically acceptable salt thereof, as claimed in claim 14.

36. The method of claim 35, wherein the administration of the prodrug is selected from the group consisting of topical, enteral, parenteral, inhalation, injection, infusion, intraarticular, intradermal, subcutaneous, intramuscular, intravenous, intraosseous, intraperitoneal, intrathecal, intracapsular, intraorbital, intracardiac, transtracheal, subcuticular, intraarticular, subcapsular, subarachnoid, intraspinal, intraventricular and intrasternal administration.

37. A method of treating, controlling, delaying or preventing in a mammalian patient in need of the treatment of one or more conditions comprising administering to the patient a diagnostically and/or therapeutically effective amount of the pharmaceutical composition of claim 34.

38. The method of claim 37, wherein administration of the pharmaceutical composition is selected from the group consisting of topical, enteral, parenteral, inhalation, injection, infusion, intraarticular, intradermal, subcutaneous, intramuscular, intravenous, intraosseous, intraperitoneal, intrathecal, intracapsular, intraorbital, intracardiac, transtracheal, subcuticular, intraarticular, subcapsular, subarachnoid, intraspinal, intraventricular and intrasternal administration.
